(19) United States
(12) Patent Application Publication
(10) Pub. No.: US 2009/0018200 A1
(43) Pub. Date: Jan. 15, 2009

(54) COMPOSITION CONTAINING A PHENANTHRENOL

(75) Inventors: Claudie WILLEMIN, Paris (FR); Geoffroy Remaut, Fresnes (FR); Angeles Fonolla Moreno, Paris (FR); Cyril Lemoine, Saint-Cyr-L'ecole (FR)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL, Paris (FR)

(21) Appl. No.: 12/172,354

(22) Filed: Jul. 14, 2008

Related U.S. Application Data
(60) Provisional application No. 60/949,295, filed on Jul. 12, 2007.

(51) Int. Cl. A61K 31/185

(52) U.S. Cl. 514/576

(57) ABSTRACT

Novel uses of phenanthrenol compounds for improving the appearance and/or texture of the skin.

### REFERENCE TO PRIOR APPLICATIONS

This application claims priority to U.S. provisional application 60/949,295 filed Jul. 12, 2007, incorporated herein by reference.

### FIELD OF THE INVENTION

The field of the invention especially concerns:
novel uses of a phenanthrenol compound;
the use, in a composition, especially a cosmetic composition, of a phenanthrenol compound in combination with at least one other skincare ingredient or active agent for improving the appearance of keratin materials, in particular the skin;
compositions comprising the combinations;
cosmetic processes using the combinations and/or the compositions.

Additional aspects and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Ageing of the contour of the lips, in particular the moustache area, is characteristic of women and especially of menopausal women, associated with an age-related hormonal deficit. This ageing is reflected especially by lengthening of the top lip area (increase in its height), by the appearance of vertical fine lines, by hollowing of the nasal groove and a reduction of the fat mass of the cheeks.

Expression wrinkles are produced by the effect of the stress exerted on the skin by the skin muscles that enable expressions. Depending on the shape of the face, the frequency of expressions and any possible tics, they may appear as early as childhood. Age, and even certain environmental factors such as exposure to sunlight, does not play a part in creating them, but may make them deeper and permanent. Expression wrinkles are characterized by the presence of grooves in the area around the orifices of the nose (nasal grooves), the mouth (perioral wrinkles and “bitterness” wrinkles) and the eyes (crow's feet wrinkles), around which lie the skin muscles, and also between the eyebrows (glabella or lion wrinkles) and on the forehead. In particular, it will be sought to prevent and/or smooth out the wrinkles of the forehead and in the area between the eyebrows.

The appearance and/or visibility of the pores is a quite recent problem, which it is sought to treat in particular in the case of individuals with dilated pores, irrespective of their origin: ethnic (e.g.: Asiatic population, Caucasian populations), excess sebum, ageing, loss of firmness, slackening, stress, fatigue, unsuitable hygiene, climatic factors, etc.

This skin imperfection, in particular on the face and especially in the T area (forehead, nose, cheeks and chin), in particular in the region of the nose and the cheeks, may be accentuated by an increase in the width of the conical portion around the pore and/or a parakeratotic state of the stratum corneum in this conical portion, which is itself associated with excessive secretion of sebum and of unsaturated fatty acids. Agents that are active on the pores improve the appearance and/or visibility of the pores, in particular tightening thereof, and thus reduce the size of dilated pores, therefore making them less visible; the grain of the skin is thus tightened and refined, to give a smoother skin surface appearance and feel and a refined grain; the skin is more radiant and/or transparent.

The wizened appearance of the skin is characterized by a change in the visual appearance, and also in the behaviour of the skin to the touch. More specifically, the skin visually takes on the appearance of cigarette paper, giving it an appearance similar to that of a sheet of papyrus. In addition, when it is pinched gently between the thumb and the index finger, the skin forms numerous fine, sharp folds having the appearance of folded paper. Finally, the feel of the skin shows that its surface parts appear to be floating on the deeper parts, giving the skin, in the very advanced stage of the wizened appearance, the appearance of crumpled paper. The wizened appearance of the skin is visible on the face and even more characteristic on the back of the hands of the elderly.

### DETAILED DESCRIPTION OF THE PREFERRED EMBODIMENTS

The expression “improving the appearance of keratin material(s)” means improving the aesthetic appearance of the material(s), for example reducing the surface irregularities of the keratin material(s) and/or improving the texture and/or the mechanical properties of the keratin material(s).

The uses according to the invention include improving the aesthetic appearance of the skin and/or its integuments, in particular improving the surface appearance and/or the texture of the skin.

The term “surface appearance” means the visual and/or tactile irregularities of the skin and/or the scalp, in particular wrinkles and fine lines, expression wrinkles, in particular on the forehead and in the space between the eyebrows, the wrinkles and/or fine lines around the mouth, and/or slackening of the contour of the lips, in particular in the moustache area (region between the top lip and the nose), the heterogeneity of the complexion (age marks or actinic lentigo), the appearance and/or visibility of the pores, the wizened appearance of the skin, skin microrelief defects such as chicken pox or acne marks, and the imperfections of greasy skin (shiny appearance, etc.).

The term “texture of the skin” refers to skin that is soft, flaccid, less firm or less elastic, or slackened skin.

According to one particular mode, the phenanthrenol compounds according to the invention are used in compositions for reducing the imperfections of greasy skin, in particular for reducing the appearance and/or visibility of the pores, and/or for tightening and/or refining the grain of the skin, and/or for reducing the shiny appearance of the skin.

Other uses according to the invention include the treatment of skin disorders associated with impairment of desquamation and/or of pigmentation of the skin. The desquamation disorders especially refer to: xerosis, acne, hyperkeratosis, psoriasis, atopy and ichthyosis.

Pigmentation disorders that may especially be mentioned include: melasma of the forearms, idiopathic melasmas, hyperpigmentations associated with pregnancy or with combination oral contraception, puva-senile lentigo, accidental hyperpigmentations, leukoderma-related hyperpigmentations and vitiligo.

The combination of a phenanthrenol compound and of at least one skincare ingredient/active agent according to the invention may be formulated in the same composition or in two different compositions, which may be applied to the skin either simultaneously or successively or sequentially over time.

The compositions according to the invention may be in the form of makeup or care products for keratin materials, in particular for the skin.

More specifically, the makeup products may be of the type such as foundations, makeup rouges, eyeshadows, concealer products or blushers, or alternatively a body makeup product or a skin colouring product.

The skincare products may be a protective, treatment or care composition for the face, for the hands, for the feet, for the major anatomical folds or for the body (for example day cream, night cream, makeup-removing cream, antisun composition, protective or care body milk, after-sun milk, skincare and/or cleansing lotion, gel or mousse, artificial tanning composition); aftershave composition.

In particular, it will be a composition for caring for and/or cleansing facial skin, in particular greasy skin.

### I—Phenanthrenols

The phenanthrenol compounds in accordance with the invention, more than one of which may be present in the invention compositions, are chosen from those corresponding to the general formula (I) below:

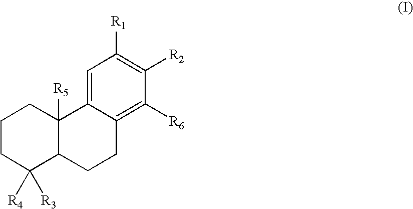

in which:
R₁, R₂ and R₆ denote, independently of each other, a group chosen from —H, —OH, a linear C₁-C₄ alkyl group, a branched C₃-C₄ alkyl group, a radical —OR with R being a C₁-C₃ alkyl radical, with the proviso that one of the groups R₁, R₂ or R₆ is a group —OH and that one of the other groups R₁, R₂ and R₆ is a linear C₁-C₄ alkyl group or a branched C₃-C₄ alkyl group;
R₃ and R₄ denote, independently of each other, a C₁-C₃ alkyl group, preferably a methyl group;
R₅ denotes a C₁-C₆ and preferably C₁-C₃ alkyl group, preferentially a methyl group;
and also the stereoisomers and diastereoisomers thereof, and mixtures thereof.

Preferably, the compounds of formula (I) that are used are those for which R₁, R₂ and R₆ have the meanings described previously; R₃, R₄ and R₅ denote a methyl group.

Preferentially, the compounds of formula (I) that are used are those for which:

R₁, R₂ and R₆ are each different and denote, independently of each other, a group chosen from —H, —OH, a branched C₃-C₄ and preferably C₃ alkyl group;
R₃, R₄ and R₅ denote a methyl group.

The phenanthrenol compounds of formula (I) that are particularly preferred are those for which R1=H; R2=OH; R₆=-i-propyl; R₃, R₄ and R₅ denote a methyl group.

The compounds of formula (I) may be obtained by synthesis or by extraction using starting materials of natural origin containing such compounds.

Advantageously, the phenanthrenol compounds of formula (I) are chosen from:

| Compound | Chemical Abstract | | | | | | | |
|---|---|---|---|---|---|---|---|---|
| No. | Name | CAS | R1 | R2 | R6 | R3 | R4 | R5 |
| 1 | 4b,5,6,7,8,8a,9,10- | 112867- | H | OH | iPr | Me | Me | Me |
| | octahydro-4b,8,8- | 87-5 | | | | | | |
| | trimethyl-1-(1-methyl- | | | | | | | |
| | ethyl)-, (4bR,8aR)- | | | | | | | |
| | rel-2-Phenanthrenol | | | | | | | |
| | (or (±)Totarol) | | | | | | | |
| 2 | 4b,5,6,7,8,8a,9,10- | 140670- | OMe | iPr | OH | Me | Me | Me |
| | octahydro-3-methoxy- | 87-7 | | | | | | |
| | 4b,8,8-trimethyl-2-(1- | | | | | | | |
| | methylethyl)-, (4bS- | | | | | | | |
| | trans)-1- | | | | | | | |
| | Phenanthrenol | | | | | | | |
| 3 | 4b,5,6,7,8,8a,9,10- | 67119- | H | iPr | OH | Me | Me | Me |
| | octahydro-4b,8,8- | 95-3 | | | | | | |
| | trimethyl-2-(1-methyl- | | | | | | | |
| | ethyl)-, (4bS,8aS)-1- | | | | | | | |
| | Phenanthrenol | | | | | | | |
| 4 | 4b,5,6,7,8,8a,9,10- | 411239- | H | OH | iPr | Me | Me | Me |
| | octahydro-4b,8,8- | 21-9 | | | | | | |
| | trimethyl-1-(1-methyl- | | | | | | | |
| | ethyl)-2-Phenanthrenol | | | | | | | |
| 5 | 3-ethyl- | 294191- | Et | OH | iPr | Me | Me | Me |
| | 4b,5,6,7,8,8a,9,10- | 34-7 | | | | | | |
| | octahydro-4b,8,8- | | | | | | | |
| | trimethyl-1-(1-methyl- | | | | | | | |
| | ethyl)-, (4bS,8aS)-2- | | | | | | | |
| | Phenanthrenol | | | | | | | |
| 6 | 4b,5,6,7,8,8a,9,10- | 130790- | H | OH | iPr | Me | Me | Me |
| | octahydro-4b,8,8-tri- | 11-3 | | | | | | |
| | methyl-1-(1-methyl- | | | | | | | |
| | ethyl)-, (4bS,8aR)-2- | | | | | | | |
| | Phenanthrenol | | | | | | | |
| | (or cis-Totarol) | | | | | | | |
| 7 | 4b,5,6,7,8,8a,9,10- | 71629- | OMe | OH | iPr | Me | Me | Me |
| | octahydro-3-methoxy- | 16-8 | | | | | | |
| | 4b,8,8-trimethyl-1-(1- | | | | | | | |
| | methylethyl)-, (4bS- | | | | | | | |
| | trans)-2-Phenanthrenol | | | | | | | |
| 8 | 4b,5,6,7,8,8a,9,10- | 63568- | OMe | OH | iPr | Me | Me | Me |
| | octahydro-3-methoxy- | 41-2 | | | | | | |
| | 4b,8,8-trimethyl-1-(1- | | | | | | | |
| | methylethyl)-, trans- | | | | | | | |
| | 2-Phenanthrenol | | | | | | | |
| | (or (±)-Dispermol) | | | | | | | |
| 9 | 4b,5,6,7,8,8a,9,10- | 1857- | iPr | OH | H | Me | Me | Me |
| | octahydro-4b,8,8- | 11-0 | | | | | | |
| | trimethyl-3-(1-methyl- | | | | | | | |
| | ethyl)-, (4bS,8aS)-2- | | | | | | | |
| | Phenanthrenol | | | | | | | |
| | (or (+)-Sempervirol) | | | | | | | |
| 10 | 4b,5,6,7,8,8a,9,10- | 17990- | iPr | OH | H | Me | Me | Me |
| | octahydro-4b,8,8- | 27-1 | | | | | | |
| | trimethyl-3-(1-methyl- | | | | | | | |
| | ethyl)-, trans-2- | | | | | | | |
| | Phenanthrenol | | | | | | | |
| | (or (±)-Sempervirol) | | | | | | | |
| 11 | 4b,5,6,7,8,8a,9,10- | 511-15-9 | H | OH | iPr | Me | Me | Me |
| | octahydro-4b,8,8- | | | | | | | |
| | trimethyl-1-(1-methyl- | | | | | | | |
| | ethyl)-, (4bS,8aS)-2- | | | | | | | |
| | Phenanthrenol | | | | | | | |
| | (or trans-Totarol) | | | | | | | |
| 12 | 4b,5,6,7,8,8a,9,10- | 304665- | OH | iPr | H | Me | Me | Me |
| | octahydro-4b,8,8- | 17-6 | | | | | | |
| | trimethyl-2-(1-methyl- | | | | | | | |
| | ethyl)-, (4bR,8aR)-3- | | | | | | | |
| | Phenanthrenol | | | | | | | |
| | (or Ferruginol) | | | | | | | |
| 13 | 4b,5,6,7,8,8a,9,10- | 117456- | OH | iPr | OMe | Me | Me | Me |
| | octahydro-1-methoxy- | 91-4 | | | | | | |
| | 4b,8,8-trimethyl-2-(1- | | | | | | | |
| | methylethyl)-, (4bS- | | | | | | | |
| | trans)-3-Phenanthrenol | | | | | | | |
| 14 | 4b,5,6,7,8,8a,9,10- | 112744- | OH | iPr | H | Me | Me | Me |
| | octahydro-4b,8,8- | 09-9 | | | | | | |
| | trimethyl-2-(1-methyl- | | | | | | | |
| | ethyl)-3-Phenanthrenol | | | | | | | |
| 15 | 4b,5,6,7,8,8a,9,10- | 41437- | OH | H | iPr | Me | Me | Me |
| | octahydro-4b,8,8- | 37-0 | | | | | | |
| | trimethyl-1-(1-methyl- | | | | | | | |
| | ethyl)-, cis-3- | | | | | | | |
| | Phenanthrenol | | | | | | | |
| 16 | 4b,5,6,7,8,8a,9,10- | 10219- | OH | iPr | H | Me | Me | Me |
| | octahydro-4b,8,8- | 82-6 | | | | | | |
| | trimethyl-2-(1-methyl- | | | | | | | |
| | ethyl)-, (4bR,8aR)- | | | | | | | |
| | rel-3-Phenanthrenol | | | | | | | |
| | (or (±)-Ferruginol) | | | | | | | |
| 17 | 4b,5,6,7,8,8a,9,10- | 514-62-5 | OH | iPr | H | Me | Me | Me |
| | octahydro-4b,8,8- | | | | | | | |
| | trimethyl-2-(1-methyl- | | | | | | | |
| | ethyl)-, (4bS,8aS)-3- | | | | | | | |
| | Phenanthrenol | | | | | | | |
| | (or (+)-Ferruginol) | | | | | | | |
| 18 | 4b,5,6,7,8,8a,9,10- | 51847- | OH | OMe | iPr | Me | Me | Me |
| | octahydro-2-methoxy- | 85-9 | | | | | | |
| | 4b,8,8-trimethyl-1-(1- | | | | | | | |
| | methylethyl)-, (4bS- | | | | | | | |
| | trans)-3-Phenanthrenol | | | | | | | |
| | (or (+)-Dispermol) | | | | | | | |
| 19 | 4b,5,6,7,8,8a,9,10- | 294191- | Me | OH | iPr | Me | Me | Me |
| | octahydro-3,4b,8,8- | 33-6 | | | | | | |
| | tetramethyl-1-(1- | | | | | | | |
| | methylethyl)-, | | | | | | | |
| | (4bS,8aS)-2- | | | | | | | |
| | Phenanthrenol | | | | | | | |
| 20 | 4b,5,6,7,8,8a,9,10- | 172139- | OH | iPr | OH | Me | Me | Me |
| | octahydro-4b,8,8- | 24-1 | | | | | | |
| | trimethyl-2-(1-methyl- | | | | | | | |
| | ethyl)-, trans-1,3- | | | | | | | |
| | Phenanthrènediol | | | | | | | |
| 21 | 4b,5,6,7,8,8a,9,10- | 140670- | OH | iPr | OH | Me | Me | Me |
| | octahydro-4b,8,8- | 86-6 | | | | | | |
| | trimethyl-2-(1-methyl- | | | | | | | |
| | ethyl)-, (4bS,8aS)- | | | | | | | |
| | 1,3-Phenanthrènediol | | | | | | | |
| 22 | 4b,5,6,7,8,8a,9,10- | 84104- | OH | OH | iPr | Me | Me | Me |
| | octahydro-4b,8,8- | 87-0 | | | | | | |
| | trimethyl-1-(1-methyl- | | | | | | | |
| | ethyl)-, (4bS,8aS)- | | | | | | | |
| | 2,3-Phenanthrènediol | | | | | | | |
| 23 | 8,11,13-trien-12-ol, | 10395- | OH | H | iPr | Me | Me | Me |
| | 14-isopropyl-Podocarpa | 60-5 | | | | | | |
| 24 | 4b-hexyl- | 108193- | OH | iPr | H | Me | Me | n- |
| | 4b,5,6,7,8,8a,9,10- | 63-1 | | | | | | hexyl |
| | octahydro-8,8- | | | | | | | |
| | dimethyl-2-(1-methyl- | | | | | | | |
| | ethyl)-, (4bS-trans)- | | | | | | | |
| | 3-Phenanthrenol | | | | | | | |
| 25 | 4b,5,6,7,8,8a,9,10- | 108193- | OH | iPr | H | Me | Me | n- |
| | octahydro-8,8- | 62-0 | | | | | | pentyl |
| | dimethyl-2-(1-methyl- | | | | | | | |
| | ethyl)-4b-pentyl-, | | | | | | | |
| | (4bS-trans)-3- | | | | | | | |
| | Phenanthrenol | | | | | | | |
| 26 | 4b-butyl- | 108193- | OH | iPr | H | Me | Me | n- |
| | 4b,5,6,7,8,8a,9,10- | 61-9 | | | | | | butyl |
| | octahydro-8,8- | | | | | | | |
| | dimethyl-2-(1-methyl- | | | | | | | |
| | ethyl)-, (4bS-trans)- | | | | | | | |
| | 3-Phenanthrenol | | | | | | | |
| 27 | 4b,5,6,7,8,8a,9,10- | 108193- | OH | iPr | H | Me | Me | n-Pr |
| | octahydro-8,8- | 60-8 | | | | | | |
| | dimethyl-2-(1-methyl- | | | | | | | |
| | ethyl)-4b-propyl-, | | | | | | | |
| | (4bS-trans)-3- | | | | | | | |
| | Phenanthrenol | | | | | | | |
| 28 | 4b-ethyl- | 108193- | OH | Et | H | Me | Me | Et |
| | 4b,5,6,7,8,8a,9,10- | 59-5 | | | | | | |
| | octahydro-8,8- | | | | | | | |
| | dimethyl-2-(1-methyl- | | | | | | | |
| | ethyl)-, (4bS-trans)- | | | | | | | |
| | 3-Phenanthrenol | | | | | | | |

Preferably, the phenanthrenol compounds of formula (I) may be chosen from:

| Compound | | | | | | | | |
|---|---|---|---|---|---|---|---|---|
| No. | Chemical Abstract Name | CAS | R1 | R2 | R6 | R3 | R4 | R5 |
| 1 | 4b,5,6,7,8,8a,9,10- | 112867- | H | OH | iPr | Me | Me | Me |
| | octahydro-4b,8,8- | 87-5 | | | | | | |
| | trimethyl-1-(1-methyl- | | | | | | | |
| | ethyl)-, (4bR,8aR)- | | | | | | | |
| | rel-2-Phenanthrenol | | | | | | | |
| | (or (±)Totarol) | | | | | | | |
| 3 | 4b,5,6,7,8,8a,9,10- | 67119- | H | iPr | OH | Me | Me | Me |
| | octahydro-4b,8,8- | 95-3 | | | | | | |
| | trimethyl-2-(1-methyl- | | | | | | | |
| | ethyl)-, (4bS,8aS)-1- | | | | | | | |
| | Phenanthrenol | | | | | | | |
| 4 | 4b,5,6,7,8,8a,9,10- | 411239- | H | OH | iPr | Me | Me | Me |
| | octahydro-4b,8,8- | 21-9 | | | | | | |
| | trimethyl-1-(1-methyl- | | | | | | | |
| | ethyl)-2-Phenanthrenol | | | | | | | |
| 6 | 4b,5,6,7,8,8a,9,10- | 130790- | H | OH | iPr | Me | Me | Me |
| | octahydro-4b,8,8- | 11-3 | | | | | | |
| | trimethyl-1-(1-methyl- | | | | | | | |
| | ethyl)-, (4bS,8aR)- | | | | | | | |
| | 2-Phenanthrenol | | | | | | | |
| | (or cis-Totarol) | | | | | | | |
| 9 | 4b,5,6,7,8,8a,9,10- | 1857- | iPr | OH | H | Me | Me | Me |
| | octahydro-4b,8,8- | 11-0 | | | | | | |
| | trimethyl-3-(1-methyl- | | | | | | | |
| | ethyl)-, (4bS,8aS)-2- | | | | | | | |
| | Phenanthrenol | | | | | | | |
| | (or (+)-Sempervirol) | | | | | | | |
| 10 | 4b,5,6,7,8,8a,9,10- | 17990- | iPr | OH | H | Me | Me | Me |
| | octahydro-4b,8,8- | 27-1 | | | | | | |
| | trimethyl-3-(1-methyl- | | | | | | | |
| | ethyl)-, trans-2- | | | | | | | |
| | Phenanthrenol | | | | | | | |
| | (or (±)-Sempervirol) | | | | | | | |
| 11 | 4b,5,6,7,8,8a,9,10- | 511-15-9 | H | OH | iPr | Me | Me | Me |
| | octahydro-4b,8,8- | | | | | | | |
| | trimethyl-1-(1-methyl- | | | | | | | |
| | ethyl)-, (4bS,8aS)-2- | | | | | | | |
| | Phenanthrenol | | | | | | | |
| | (or trans-Totarol) | | | | | | | |
| 12 | 4b,5,6,7,8,8a,9,10- | 304665- | OH | iPr | H | Me | Me | Me |
| | octahydro-4b,8,8- | 17-6 | | | | | | |
| | trimethyl-2-(1-methyl- | | | | | | | |
| | ethyl)-, (4bR,8aR)-3- | | | | | | | |
| | Phenanthrenol | | | | | | | |
| | (or Ferruginol) | | | | | | | |
| 14 | 4b,5,6,7,8,8a,9,10- | 112744- | OH | iPr | H | Me | Me | Me |
| | octahydro-4b,8,8- | 09-9 | | | | | | |
| | trimethyl-2-(1-methyl- | | | | | | | |
| | ethyl)-3-Phenanthrenol | | | | | | | |
| 15 | 4b,5,6,7,8,8a,9,10- | 41437- | OH | H | iPr | Me | Me | Me |
| | octahydro-4b,8,8- | 37-0 | | | | | | |
| | trimethyl-1-(1-methyl- | | | | | | | |
| | ethyl)-, cis-3- | | | | | | | |
| | Phenanthrenol | | | | | | | |
| 16 | 4b,5,6,7,8,8a,9,10- | 10219- | OH | iPr | H | Me | Me | Me |
| | octahydro-4b,8,8- | 82-6 | | | | | | |
| | trimethyl-2-(1-methyl- | | | | | | | |
| | ethyl)-, (4bR,8aR)- | | | | | | | |
| | rel-3-Phenanthrenol | | | | | | | |
| | (or (±)-Ferruginol) | | | | | | | |
| 17 | 4b,5,6,7,8,8a,9,10- | 514-62-5 | OH | iPr | H | Me | Me | Me |
| | octahydro-4b,8,8- | | | | | | | |
| | trimethyl-2-(1-methyl- | | | | | | | |
| | ethyl)-, (4bS,8aS)-3- | | | | | | | |
| | Phenanthrenol | | | | | | | |
| | (or (+)-Ferruginol) | | | | | | | |
| 19 | 4b,5,6,7,8,8a,9,10- | 294191- | Me | OH | iPr | Me | Me | Me |
| | octahydro-3,4b,8,8- | 33-6 | | | | | | |
| | tetramethyl-1-(1- | | | | | | | |
| | methylethyl)-, | | | | | | | |
| | (4bS,8aS)-2- | | | | | | | |
| | Phenanthrenol | | | | | | | |
| 23 | 8,11,13-trien-12-ol, | 10395- | OH | H | iPr | Me | Me | Me |
| | 14-isopropyl-Podocarpa | 60-5 | | | | | | |

Preferentially, the phenanthrenol compounds of formula (I) may be chosen from:

| Com- | | | | | | | | |
|---|---|---|---|---|---|---|---|---|
| pound | Chemical | | | | | | | |
| No. | Abstract Name | CAS | R1 | R2 | R6 | R3 | R4 | R5 |
| 1 | 4b,5,6,7,8,8a,9,10- | 112867- | H | OH | iPr | Me | Me | Me |
| | octahydro-4b,8,8- | 87-5 | | | | | | |
| | trimethyl-1-(1-methyl- | | | | | | | |
| | ethyl)-, (4bR,8aR)- | | | | | | | |
| | rel-2-Phenanthrenol | | | | | | | |
| | (or (±)Totarol) | | | | | | | |
| 4 | 4b,5,6,7,8,8a,9,10- | 411239- | H | OH | iPr | Me | Me | Me |
| | octahydro-4b,8,8- | 21-9 | | | | | | |
| | trimethyl-1-(1-methyl- | | | | | | | |
| | ethyl)-2-Phenanthrenol | | | | | | | |
| 6 | 4b,5,6,7,8,8a,9,10- | 130790- | H | OH | iPr | Me | Me | Me |
| | octahydro-4b,8,8- | 11-3 | | | | | | |
| | trimethyl-1-(1-methyl- | | | | | | | |
| | ethyl)-, (4bS,8aR)- | | | | | | | |
| | 2-Phenanthrenol | | | | | | | |
| | (or cis-Totarol) | | | | | | | |
| 11 | 4b,5,6,7,8,8a,9,10- | 511-15- | H | OH | iPr | Me | Me | Me |
| | octahydro-4b,8,8- | 9 | | | | | | |
| | trimethyl-1-(1-methyl- | | | | | | | |
| | ethyl)-, (4bS,8aS)-2- | | | | | | | |
| | Phenanthrenol | | | | | | | |
| | (or trans-Totarol) | | | | | | | |

The compound of structure (I′) below is particularly preferred:

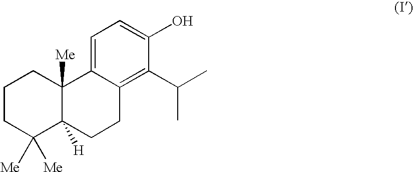

corresponding to 4b,5,6,7,8,8a,9,10-octahydro-4-b,8,8-trimethyl-1-(1-methylethyl), (4bR,8aR)-rel-2-phenanthrenol (or (±)-Totarol); (compound 1).

It is especially possible to use a purified extract of totara wood (Podocarpus totara) in powder form, obtained by supercritical CO₂ extraction, sold under the name Totarol by the company Mende-Dek Limited; this extract contains from 50% to 65% of totarol (totara-8,11,13-trien-13-ol).

The phenanthrenol compounds of formula (I) may preferably be present in the compositions according to the invention in a content of from, e.g., 0.001% to 30% by weight and more preferably ranging for example from 0.01% to 20% by weight relative to the total weight of the composition, including all values and sub-ranges between stated values as if written out.

II—Cosmetic and/or Dermatological Ingredients and/or Active Agents

The ingredients preferably used herein are those that promote the solubilization, stabilization and/or activity of the phenanthrenol compound.

According to the invention, the expression “ingredient that promotes the stabilization of the (invention) phenanthrenol compounds” means an ingredient that makes it possible either (i) to stabilize the phenanthrenol compound, or (ii) to stabilize the physiologically acceptable medium in which the phenanthrenol compound is present.

A—Ingredients that Promote the Solubilization of the Phenanthrenol Compounds

As ingredients that promote the solubilization of the invention phenanthrenol compounds, mention may be made especially of:

(i) lipophilic derivatives of amino acids, especially such as those described in patent application EP 1 269 986, which is incorporated herein by reference.

In particular, lipophilic derivatives of amino acids that may be used include those of formula

R′₁(CO)N(R′₂)CH(R′₃)(CH₂)n(CO)OR′₄

in which:
n is an integer equal to 0, 1 or 2,
R′₁ represents a linear or branched C₅-C₂₁ alkyl or alkenyl radical,
R′₂ represents a hydrogen atom or a C₁-C₃ alkyl group,
R′₃ represents a radical chosen from the group formed by a hydrogen atom, a methyl group, an ethyl group and a linear or branched C₃ or C₄ alkyl radical,
R′₄ represents a linear or branched C₁-C₁₀ alkyl or C₂-C₁₀ alkenyl radical, or a sterol residue.

A preferred lipophilic amino acid derivative is isopropyl N-lauroylsarcosinate, sold especially by Ajinomoto under the name Eldew SL 205.

(ii) fatty alcohols containing from 8 to 26 carbon atoms, such as lauryl alcohol, cetyl alcohol, myristyl alcohol, stearyl alcohol, palmityl alcohol, oleyl alcohol, linoleyl alcohol and 2-octyldodecanol, and mixtures thereof, such as cetearyl alcohol (mixture of cetyl alcohol and of stearyl alcohol). Preferably, the 2-octyldodecanol sold under the name Eutanol G by the company Cognis or under the name Isofol 20 N/F by the company Sasol will be used.
(iii) ethers such as dicaprylyl ether (CTFA name: Dicaprylyl ether); C₁₂-C₁₅ fatty alkyl benzoates (Finsolv TN from Finetex). The dicaprylyl carbonate sold under the name Cetiol CC by the company Cognis will be used in particular.
(iv) dimethyl isosorbide.

According to one particular mode, to increase the solubilization effect of the above ingredients, liposoluble UV-screening agents such as those described in L'Oréal patent application EP 1 221 933 may also be added.

Mention may be made especially of:

(1) salicylic acid derivatives chosen from homomethyl salicylate, 2-ethylhexyl salicylate, triethanolamine salicylate and 4-isopropylbenzyl salicylate;
(2) cinnamic acid derivatives such as isopentyl 4-methoxycinnamate, 2-ethylhexyl 4-methoxycinnamate, methyl diisopropyl cinnamate, isoamyl 4-methoxycinnamate and diethanolamine 4-methoxycinnamate;
(3) liquid β,β′-diphenylacrylate derivatives;
(4) para-aminobenzoic acid derivatives such as 2-ethylhexyl p-dimethylaminobenzoate and glyceryl p-aminobenzoate;
(5) liposoluble benzophenone derivatives;
(6) the benzotriazole silicones described especially in patent application EP-A-0 392 883;
(7) the silicon derivatives of N-substituted benzimidazolyl-benzazoles or of benzofuryl-benzazoles described especially in patent application EP-1 028 120; and
(8) mixtures thereof.

Among the cinnamic acid derivatives mentioned above, it is most particularly preferred according to the present invention to use 2-ethylhexyl p-methoxycinnamate, or octyl methoxycinnamate, sold especially under the trade name Parsol MCX by the company Givaudan.

Among the salicylic acid derivatives, octyl salicylate sold especially under the trade name Uvinul 0-18 by the company BASF will preferentially be used.

In addition, the weight amount of liposoluble UV-screening agent advantageously represents from 0.5% to 20% and better still from 0.1% to 10% of the total weight of the composition.

The invention thus relates to a composition comprising at least one phenanthrenol compound and an ingredient that promotes the solubilization of the at least one phenanthrenol compound chosen from: a lipophilic amino acid derivative, fatty alcohols containing from 8 to 26 carbon atoms, dicaprylyl ethers and c12-C15 fatty alkyl benzoates, and mixtures thereof.

In particular, the ingredient that promotes the solubilization of the phenanthrenol compounds is an ingredient chosen from isopropyl n-lauroylsarcosinate, 2-octyldodecanol, dicaprylyl ether, a C12-C15 fatty alkyl benzoate and dimethyl isosorbide, and mixtures thereof.

A liposoluble UV-screening agent chosen from 2-ethylhexyl p-methoxycinnamate, or octyl methoxycinnamate, octyl salicylate and 2-(2H-benzotriazol-2-yl)-4-methyl-6-[2-methyl-3-[1,3,3,3-tetramethyl-1-[(trimethylsilyl)oxy]disiloxanyl]propynyl]phenol may also be added.

### B—Ingredients Promoting the Stabilization of the Phenanthrenol Compounds

According to the invention, the expression “ingredient that promotes the stabilization the invention phenanthrenol compounds” means an ingredient that makes it possible either (i) to stabilize the phenanthrenol compounds, or (ii) to stabilize the physiologically acceptable medium in which the phenanthrenol compound is present.

In particular, in case (ii), the ingredient that promotes the stabilization of the phenanthrenol compounds may especially be present in a particular galenical form that contributes towards stabilizing the phenanthrenol compound in the physiologically acceptable medium.

As ingredients for stabilizing the phenanthrenol compounds that may be used in the compositions of the invention, mention may be made especially of:
(a) block polymers and/or copolymers;
(b) amphiphilic lipids of ionic or nonionic type present in the form of vesicles in dispersion;
(c) constituent polymers of nanoparticles, in particular of nanospheres or nanocapsules;
(d) constituent polymers of microparticles;
(e) polymers and/or surfactants forming nanoemulsions;
(f) polymers in the form of thin films;
(g) polyolefin emulsifiers with a polar portion, the composition being in the form of a water-in-oil emulsion;
(h) amphiphilic polymers comprising 2-acrylamido-2-methylpropanesulfonic acid (AMPS) units.

According to one particular mode, the composition according to the invention comprises at least one constituent polymer of microparticles (d).

(a) Block Polymers and/or Copolymers

It is known practice to encapsulate active agents in micelles of block copolymers, for example poly(ethylene oxide-propylene oxide) diblock or triblock copolymers.

Advantageously, a block amphiphilic copolymer comprising at least one nonionic hydrophilic polymeric block and at least one particular hydrophobic polymeric block will be used in the composition of the invention.

These block amphiphilic copolymers are especially described in patent application EP 1 555 984, which is incorporated herein by reference.

The molecular weight of the block copolymer is generally between 1000 and 100 000.

In particular, the weight ratio of the ionic or nonionic hydrophilic polymeric block(s) to the hydrophobic polymeric block(s) is between 1/100 and 50/1.

The hydrophobic polymeric block is especially chosen from:
styrene and derivatives thereof such as 4-butylstyrene,
alkylene oxides containing more than 4 carbon atoms and preferably from 4 to 6 carbon atoms,
hydrophobic vinyl monomers of formula (A) below:

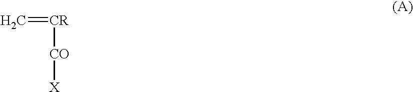

in which:
   R is chosen from H and —CH₃,
   X is chosen from alkyl oxides of —OR′ type in which R′ is a linear or branched, saturated or unsaturated hydrocarbon-based radical containing from 1 to 22 carbon atoms.

Preferably, the hydrophobic polymeric block is obtained from one or more hydrophobic monomers chosen from methyl methacrylate, ethyl methacrylate, n-butyl (meth)acrylate, tert-butyl (meth)acrylate and cyclohexyl acrylate.

In particular, the hydrophobic polymeric block is chosen from polystyrene, poly(tert-butylstyrene), polymethyl methacrylate, polymethyl acrylate, polybutyl methacrylate and poly(C₃-C₆ alkylene oxides).

Preferably, the nonionic hydrophilic polymeric block is chosen from polyethylene oxides.

Preferably, the block copolymer is chosen from the following block copolymers:
polystyrene/polyoxyethylene
polymethyl methacrylate/polyoxyethylene
polybutyl methacrylate/polyoxyethylene
polyoxyethylene/polyoxybutylene/polyoxyethylene.

### (b) Amphiphilic Lipids of Ionic or Nonionic Type:

According to another embodiment of the invention, the phenanthrenol compounds are combined with ionic or nonionic amphiphilic lipids present in the form of vesicles of ionic type (e.g.: liposomes) and/or nonionic type (e.g.: niosomes) dispersed in the physiologically acceptable medium of the composition, in particular as an aqueous dispersion.

The presence of these vesicles contributes towards stabilizing the phenanthrenol compounds in the physiologically acceptable medium of the composition.

These lipid vesicles may have an aqueous core or an oily core.

Lipid vesicles with an oily core will preferentially be used.

According to the invention, the term “vesicles” means any particular structure comprising, on the one hand, a membrane or “liquid phase” constituted by one or more concentric leaflets, these leaflets comprising one or more bimolecular layers based on ionic or nonionic amphiphilic lipids, and, on the other hand, an aqueous or oily phase encapsulated by this lipid phase. For the purposes of the invention, liposomes and niosomes especially constitute such vesicles.

Niosomes are vesicles prepared from nonionic amphiphilic lipids. Reference may be made especially to the description of patent FR 8 907 947, which is incorporated into the present invention by reference.

Nonionic amphiphilic lipids that may especially be mentioned include optionally oxyethylenated alkyl- or polyalkyl esters of polyol, and optionally oxyethylenated polyol ethers, with a melting point of at least 40° C.

Liposomes are vesicles prepared from ionic amphiphilic lipids. These vesicles are particles formed from a membrane constituted by one or more concentric leaflets, these leaflets comprising one or more bimolecular layers of amphiphilic lipids encapsulated in an aqueous or oily phase. The aqueous phase may contain water-soluble active substances and the bimolecular layers of amphiphilic lipids may contain lipophilic active substances. These vesicles generally have a mean diameter of between 10 and 5000 nanometres.

The ionic amphiphilic lipids may be anionic amphiphilic lipids or cationic amphiphilic lipids.

Examples of anionic amphiphilic lipids that may especially be mentioned include:
neutralized ionic lipids, preferably chosen from alkali metal salts of dicetyl phosphate, and of dimyristyl phosphate, in particular the sodium and potassium salts, alkali metal salts of phosphatidic acid, in particular the sodium salt, alkali metal salts of cholesteryl sulfate, in particular the sodium salt, alkali metal salts of cholesteryl phosphate, in particular the sodium salt, and salts of lipoamino acids such as monosodium and disodium acylglutamates, more particularly the disodium salt of N-stearoyl-L-glutamic acid sold under the name Acylglutamate HS21 by the company Ajinomoto,
amphoteric lipids, in particular pure soybean phosphatidylethanolamine,
alkylsulfonic derivatives.

Cationic amphiphilic lipids that may especially be used include quaternary ammonium salts and fatty amines and salts thereof.

It is advantageously possible to use “double-liposome” compositions for the simultaneous treatment of the surface and deep layers of the skin, comprising a first dispersion of lipid vesicles that are capable of penetrating into the deep layers of the skin and containing at least one active agent capable of treating these deep layers, and a second dispersion of lipid vesicles capable of penetrating into the surface layers of the skin and containing at least one active agent capable of treating these surface layers. Such a system is described in patent EP 0 661 035, which is incorporated herein by reference.

Oleosomes are oily globules provided with a lamellar liquid crystal coating, dispersed in an aqueous phase, with a mean diameter generally less than 500 nanometres.

As an example of a formulation in oleosomes, reference may be made especially to patent EP 0 641 557, which is incorporated into the present invention by reference.

### (c) Constituent Polymers of Nanoparticles:

According to one alternative, the phenanthrenol compounds may be combined with small particles, especially known as nanoparticles.

These may be solid particles constituted by the combination of the phenanthrenol compounds and of at least one polymer.

The polymer contributes towards stabilizing the phenanthrenol compounds in the physiologically acceptable medium of the composition.

The term “nanoparticles” mainly includes two different systems: “nanospheres” constituted by a polymer matrix in which the phenanthrenol compound is absorbed and/or adsorbed and/or mixed, and also “nanocapsules” having a structure of core-shell type, i.e. a structure constituted by a lipid core that is liquid at room temperature containing the phenanthrenol compound in dissolved form, this core being encapsulated in a continuous protective shell that is insoluble in the medium.

Nanocapsules will preferably be used.

Reference may be made, for example, to the description of patent application EP 1 414 390, which is incorporated herein by reference.

Nanospheres generally have a mean size of between 50 and 500 nm.

Nanocapsules are generally of small size so as to obtain optimum bioavailability of the phenanthrenol compounds.

Preferentially, the size of these nanocapsules is between 10 nm and 1000 nm and more particularly between 30 nm and 500 nm.

It is especially possible to use polymers in the form of nanocapsules, as described in patent application EP-0 274 961, nanocapsules provided with a lamellar coating, described in patent application EP-0 780 115, nanocapsules whose water-insoluble continuous polymer shell is constituted by polyesters, as described in patent applications EP-1 025 901, FR-2 787 730 and EP-1 034 839, or alternatively the biodegradable nanocapsules described in patent application FR-2 659 554, or the non-biodegradable nanocapsules described in patent application WO-93/05753.

The nanocapsules made of biodegradable polymers penetrate the skin and degrade in the epidermis under the action of the enzymes present therein, whereas the nanocapsules made of non-biodegradable polymers only penetrate the surface layers of the stratum corneum and are naturally removed during skin renewal.

As constituent polymers of nanocapsules that may be used in the compositions of the invention in combination with the phenanthrenol compounds, mention may be made especially of poly-L- and DL-lactides and polycaprolactones, polyglycolides and copolymers thereof, polymers derived from the polymerization of an alkyl cyanoacrylate (the alkyl chain containing from 2 to 6 carbon atoms); synthetic or natural water-dispersible anionic polymers; polyesters of the poly(alkylene adipate) type; dendritic polymers; copolymers of vinyl chloride and of vinyl acetate, copolymers of methacrylic acid and of methacrylic acid methyl ester, polyvinyl acetophthalate, cellulose acetophthalate, copolymers of polyvinylpyrrolidone-crosslinked vinyl acetate, polyethylene vinyl acetates, polyacrylonitriles, polyacrylamides, polyethylene glycols, polyamides, polyethylenes, polypropylenes and organopolysiloxanes.

Polycaprolactones will preferably be used.

### (d) Constituent Polymers of Microparticles:

According to one particular embodiment of the invention, which is advantageous especially for caring for greasy skin, the phenanthrenol compounds will be combined with constituent polymers of microparticles.

These polymers contribute towards stabilizing the phenanthrenol compound in the physiologically acceptable medium of the composition.

The term “microparticles” especially covers “porous particles” and in particular “microspheres”.

The term “porous particles” denotes particles with a structure comprising pores. This porous structure may at least partly enable the incorporation of one or more active agents into the particles.

Reference may be made especially to patent application EP 1 637 124, which is incorporated herein by reference. Porous organic particles (e.g.: Nylon) or porous inorganic particles (e.g.: silica) described in patent application EP 1 493 433 may advantageously be used for stabilizing the phenanthrenol compound, optionally in combination with another cosmetic active agent.

The particles are also mentioned in EP 1 723 949 for the follicles to be targeted.

The structure of the particles may be of matrix type like a sponge. It may also be of vesicular type, i.e. the particle has an inner cavity delimited by a porous wall. The porosity associated with the size of the particles is characterized quantitatively by their specific surface area.

Porous particles with a specific surface area, measured according to the BET method, of greater than or equal to 1 m²/g will especially be used. The BET method (Brunauer-Emmet-Teller) is a method that is well known to those skilled in the art. It is described especially in the “Journal of the American Chemical Society”, vol. 60, page 309, February 1938, and corresponds to international standard ISO 5794/1 (appendix D). The specific surface area measured according to the BET method corresponds to the total specific surface area, i.e. it includes the area formed by the pores.

According to one particular embodiment, the particles of the invention have a specific surface area measured by BET ranging for example especially from 2.5 to 100 m²/g.

The porous particles that may be used in the compositions of the invention are generally individualized particles. The term “individualized particles” denotes particles that are not grouped in the form of an aggregate or an agglomerate. These particles in particular have a mass per unit volume of greater than or equal to 0.15 g/cm³ and especially ranging for example from 0.2 to 5 g/cm³.

These particles preferably have a mean volume diameter of less than or equal to 10 μm. Specifically, such particles may penetrate the sebaceous follicles by application of a mechanical force. This mechanical force generally originates from massaging, which, besides the force it exerts, generates a pump effect in the follicles. The particles thus gradually reach the follicular canal, in which they may absorb sebum and, where appropriate, release the active compound they bear. The constituent material of the particles is then rejected by means of the flow of sebum and/or the growth of the hair stalk, thus making it possible to avoid any possible adverse reaction of the body with respect to this material.

Porous particles, which are especially spherical, whose number-average size may range from 0.1 to 15 μm, especially from 0.1 to 20 μm and most particularly from 0.5 to 10 μm, will be used in particular.

Polyamide particles, in particular made of Nylon 6, Nylon 6-6, Nylon 12 or Nylon 6-12, such as those sold by the company Atofina under the generic name Orgasol, may be used as preferred porous particles.

This encapsulation system that allows the follicles to be targeted is particularly advantageous in compositions for treating greasy skin.

According to one particular mode of the invention, the porous particles also contain other active agents in combination with the phenanthrenols.

In particular, porous particles filled with a mixture of phenanthrenol compound (e.g.: Totarol®), desquamating agent and calmative will be used.

The desquamating agent is in particular 5-n-octanoylsalicylic acid.

The calmative is in particular a potassium salt of β-glycyrrhetinic acid.

Such Nylon-12 particles filled with a mixture of Totarol® (2.5%), 5-n-octanoylsalicylic acid (5%) and β-glycyrrhetinic acid (2.5% by weight relative to the total weight of the starting material) under the name Mexoryl SBL® from the company Chimex. (NB: check whether it is OK to mention them)

(e) Polymers and/or Surfactants Forming Nanoemulsions:

According to another embodiment of the invention, the phenanthrenol compounds are combined in the composition with particular polymers and/or surfactants, the composition being in the form of a nanoemulsion.

These polymers and/or surfactants contribute towards stabilizing the phenanthrenol compounds in the physiologically acceptable medium of the composition.

Nanoemulsions are generally oil-in-water emulsions whose oil globules have a very fine particle size, i.e. a number-average size of less than 100 nanometres (nm).

Reference may be made especially to the description of patent EP 0 728 460, which is incorporated herein by reference.

The nanoemulsions may be stabilized with a lamellar liquid-crystal coating obtained by combining a hydrophilic surfactant and a lipophilic surfactant.

Advantageously, the particular polymers allowing the preparation of nanoemulsions may be chosen from:
hydrophobic-chain anionic polymers, as described in patent EP 116 005, which is incorporated herein by reference; and/or
water-soluble nonionic polymers, as described in patent EP 1 172 077, which is incorporated herein by reference.

The particular surfactants allowing the preparation of nanoemulsions may especially be a ternary surfactant system including a mixture of nonionic surfactants and an ionic surfactant, as described in patent EP 1 353 629, which is incorporated herein by reference.

In particular, the hydrophobic-chain anionic polymer comprises hydrophobic chains chosen from linear or branched, saturated or unsaturated hydrocarbon-based chains containing from 6 to 30 carbon atoms, cycloaliphatic divalent groups and aromatic divalent groups, and preferably chosen from alkyl, arylalkyl, alkylaryl, alkylene, methylenedicyclohexyl, isophorone and phenylene chains.

The anionic polymer is especially chosen from acrylic or methacrylic acid copolymers, and 2-acrylamido-2-methylpropanesulfonic acid copolymers, and mixtures thereof.

Preferably, the anionic polymer is obtained by copolymerization of a monomer (a) chosen from α,β-ethylenically unsaturated carboxylic acids (monomer a′) and 2-acrylamido-2-methylpropanesulfonic acid (monomer a″), with a non-surfactant ethylenically unsaturated monomer (b) different from (a) and/or an ethylenically unsaturated monomer (c) resulting from the reaction of an α,β-ethylenically unsaturated acrylic monomer or a monoethylenically unsaturated isocyanate monomer with a monohydric nonionic amphiphilic component or with a primary or secondary fatty amine.

Advantageously, the anionic polymer is an acrylic terpolymer obtained from (a) an α,β-ethylenically unsaturated carboxylic acid, (b) a non-surfactant ethylenically unsaturated monomer different from (a), and (c) a nonionic urethane monomer that is the product of reaction of a monohydric nonionic amphiphilic compound with a monoethylenically unsaturated isocyanate.

For example, the anionic polymer is chosen from acrylic acid/ethyl acrylate/alkyl acrylate terpolymer, acrylates/Steareth-20 methacrylate copolymer, (meth)acrylic acid/ethyl acrylate/oxyethylenated (25 EO) behenyl methacrylate terpolymer, acrylic acid/oxyethylenated (20 EO) monocetyl itaconate copolymer, acrylic acid/oxyethylenated (20 EO) monostearyl itaconate copolymer, the copolymer of acrylates/polyoxyethylenated (25 EO) acrylate modified with C₁₂-C₂₄ alcohols, methacrylic acid/methyl acrylate/ethoxylated behenyl dimethyl-meta-isopropenyl benzylisocyanate terpolymer, and mixtures thereof.

In particular, the water-soluble nonionic polymer may be chosen from ethylene oxide homopolymers and copolymers; polyvinyl alcohols; vinylpyrrolidone homopolymers and copolymers; vinylcaprolactam homopolymers and copolymers; polyvinyl methyl ether homopolymers and copolymers; neutral acrylic homopolymers and copolymers; C₁-C₂-alkyl-celluloses and derivatives thereof; C₁-C₃-alkyl-guars or C₁-C₃-hydroxyalkyl-guars.

The ternary surfactant system may especially comprise:

(a) a mixture of at least two nonionic surfactants comprising at least one ethoxylated fatty ester containing 8 to 100 (especially 10 to 80, and better still 40) ethylene oxide units and at least one fatty acid ester of sorbitan; and
(b) at least one ionic surfactant chosen from alkali metal salts of cetyl phosphate and alkali metal salts of palmitoyl sarcosinate.

The ethoxylated fatty ester is preferably polyethylene glycol 40 EO stearate and the fatty acid ester of sorbitan is preferably sorbitan tristearate.

The anionic surfactant is especially chosen from potassium cetyl phosphate and sodium palmitoyl sarcosinate, and mixtures thereof.

### (f) Polymers in the Form of Thin Films:

According to another embodiment of the invention, the phenanthrenol compounds are combined with at least one water-soluble or water-dispersible polymer in the form of a thin film.

The composition is thus in the form of a thin film.

This water-soluble or water-dispersible polymer contributes towards stabilizing the phenanthrenol compounds in the physiologically acceptable medium of the composition.

In the present patent application, the term “film” means a thin, manipulable solid. The term “thin” refers to a solid with a maximum thickness of 1000 μm. This film generally has a suitable size to be able to be readily manipulated by the user. It may be square, rectangular or disc-shaped, or may have any other shape. Each film generally has a thickness of from 10 μm to 1000 μm, preferably from 20 to 500 μm and better still from 50 to 300 μm. It may have an area of from 0.25 to 25 cm² and preferably from 2 to 10 cm².

These thin films generally contain less than 10% by weight and preferably less than 5% by weight of water relative to the total weight of the film, and more preferably do not contain water.

Such films are described in patent application EP 1 588 694, which is incorporated herein by reference.

The thin film comprises a water-soluble or water-dispersible polymer that may be chosen from: (1) polymers of protein type, such as wheat or soybean protein; keratin, for example keratin hydrolysates and sulfonic keratins; casein; albumin; collagen; glutelin; glucagon; gluten; zein; gelatins and derivatives thereof; (2) polymers derived from chitin or from chitosan, such as anionic, cationic, amphoteric or nonionic chitin or chitosan polymers; (3) polysaccharide polymers such as, especially, (i) cellulose-based polymers, for instance hydroxyethylcellulose, hydroxypropylcellulose, hydroxypropylmethylcellulose, methylcellulose, ethylhydroxyethylcellulose, carboxymethylcellulose and quaternized cellulose derivatives; and (ii) starches and derivatives thereof; (4) acrylic polymers or copolymers such as polyacrylates, polymethacrylates and copolymers thereof; (5) vinyl polymers such as polyvinylpyrrolidones, copolymers of methyl vinyl ether and of maleic anhydride, the copolymer of vinyl acetate and of crotonic acid, copolymers of vinylpyrrolidone and of vinyl acetate, copolymers of vinylpyrrolidone and of caprolactam, polyvinyl alcohols; (6) polymers of natural origin, which are optionally modified, such as gum arabic, guar gum, xanthan derivatives or karaya gum; alginates, carrageenans, ulvanes and other algal colloids; glycosaminoglycans, hyaluronic acid and its derivatives; shellac, sandarac gum, dammar resins, elemi gums and copal resins; deoxyribonucleic acid; mucopolysaccharides such as hyaluronic acid, chondroitin sulfate; and mixtures of these polymers.

Water-soluble polymers that may also be mentioned include caprolactams, pullulan, pectin, mannan and galactomannans, and glucomannans, and derivatives thereof.

Preferably, the water-soluble polymer may be a cellulose-based polymer, in particular hydroxypropylcellulose or hydroxypropyl methylcellulose, or alternatively an alginate, especially sodium alginate.

The composition according to the invention may be in the form of an emulsion and may also be specific emulsifiers capable of stabilizing the phenanthrenol compounds and of advantageously imparting noteworthy sensory properties such as a light, fresh feel.

Examples of particular emulsifiers that may especially be mentioned include:
polyolefin emulsifiers with a polar portion, in a water-in-oil emulsion;
amphiphilic polymers comprising 2-acrylamido-2-methylpropanesulfonic acid (AMPS) units in an oil-in-water emulsion.

### (g) Polyolefin Emulsifiers Containing a Polar Portion:

The invention thus also relates to a composition in the form of a water-in-oil emulsion comprising at least one phenanthrenol compound (I) or (II) and at least one polyolefin with a polar portion.

The polyolefins with a polar portion contribute towards stabilizing the phenanthrenol compound in the physiologically acceptable medium of the composition.

The polyolefins with polar portion(s) used in the composition of the invention are generally constituted by a polyolefinic apolar portion and by at least one polar portion. The polyolefinic apolar portion comprises at least 40 carbon atoms and preferably from 60 to 700 carbon atoms. This apolar portion may be chosen from polyolefins such as oligomers, polymers and/or copolymers of ethylene, propylene, 1-butene, isobutene, 1-pentene, 2-methyl-1-butene, 3-methyl-1-butene, 1-hexene, 1-heptene 1-octene, 1-decene, 1-undecene, 1-dodecene, 1-tridecene, 1-tetradecene, 1-pentadecene, 1-hexadecene, 1-heptadecene and 1-octadecene. These polyolefins are hydrogenated or non-hydrogenated.

The polar portion of the polyolefins with a polar portion may be anionic, cationic, nonionic, zwitterionic or amphoteric. It is constituted, for example, by polyalkylene (especially polyoxyethylene) glycols or polyalkyleneimines, or alternatively by carboxylic or dicarboxylic acids, or anhydrides thereof or derivatives thereof such as esters thereof, amides thereof and salts thereof, and mixtures thereof. The polyolefins with a carboxylic acid polar portion may be derived, for example, from the reaction between the polyolefins and at least one carboxylic acid or anhydride that is optionally totally or partially salified, chosen from the group comprising succinic acid or anhydride, maleic acid, maleic anhydride, fumaric acid, itaconic acid, citraconic acid (or methylmaleic acid), mesaconic acid (or methylfumaric acid) and aconitic acid, and ester or amide derivatives thereof, and mixtures thereof.

Preferably, the polar portion of the polyolefin is chosen from the group comprising polyoxyethylene, succinic acid or anhydride, esters or amides of succinic acid or anhydride, alkali metal or alkaline-earth metal or organic salts of succinic acid or anhydride, or partial salts of succinic acid or anhydride monoesters or monoamides.

The polyolefins with a polar portion that are preferred in the compositions of the invention are polyolefins with an optionally modified succinic end group, as described in patent EP 1 172 089, which is incorporated herein by reference.

Polyolefins with a succinic end group that may especially be mentioned include polyisobutylenes with an optionally modified esterified succinic end group, especially esterified with diethanolamine, and salts thereof, especially the diethanolamine salts, such as the products sold under the names Lubrizol® 2724, Lubrizol® 2722 and Lubrizol® 5603 by the company the Lubrizol.

Another polyolefin with a polar portion that is particularly preferred is an ester of poly(diethanolaminoethyl isobutenyl succinate) and of triethanolamine. This product is sold, for example, under the name Chemcinnate® 2000 by the company Chemron.

A polyolefin with a polar portion that may also be used is a poly(glyceryl isobutenyl succinate) ester, especially the product sold under the name Chemcinnate® 1000 AF by the company Chemron.

### (h) Amphiphilic Polymers Comprising AMPS Units:

According to another embodiment of the invention, the composition according to the invention is in the form of an oil-in-water emulsion and comprises at least one phenanthrenol compound and at least one amphiphilic polymer comprising 2-acrylamido-2-methylpropanesulfonic acid (AMPS) units.

This amphiphilic polymer comprising 2-acrylamido-2-methylpropanesulfonic acid (AMPS) units contributes towards stabilizing the phenanthrenol compound in the physiologically acceptable medium of the composition.

As amphiphilic polymer comprising 2-acrylamido-2-methylpropanesulfonic acid (AMPS) units that may be used in the compositions of the invention, mention may be made of those described in patent application EP 1 466 587, which is incorporated herein by reference.

Preferred amphiphilic polymers that may be mentioned include copolymers of AMPS and of oxyethylenated C₁₂-C₁₄ alkyl methacrylate, especially comprising from 7 to 23 oxyethylene groups.

To improve the sensory properties of the composition, such as a fresh effect and a feel that is not tacky or greasy, in particular for oil-in-water emulsions, tetrapolymers may also be used.

The tetrapolymer used according to the invention comprises as monomers methacrylic acid, methyl methacrylate, butyl acrylate and a C₁₆-C₂₀ alkyl (meth)acrylate.

Tetrapolymers as defined previously, and a process for preparing them, are described in particular in patent application US 2003/0 021 847.

These tetrapolymers may be prepared by emulsion polymerization of the monomers indicated above in the presence of a free-radical initiator, such as hydrogen peroxide, tert-butyl hydroperoxide or sodium, potassium, lithium or ammonium persulfate, the initiator being optionally combined with a reducing agent to form a redox system and with a catalyst constituted by a transition metal such as a copper or iron salt. The reaction may be performed, for example, at a temperature of between 10 and 120° C. and preferably in the region of 85° C., for a time of approximately three hours.

A tetrapolymer of this type is in particular available from the company Röhm & Haas under the trade name Allianz OPT in the form of a water-glycol dispersion containing 48% active material.

The amount of tetrapolymer (as active material) in the composition according to the invention may range, for example, from 0.1% to 5% by weight, preferably from 0.2% to 5% by weight, better still from 0.2% to 2% by weight and even better still from 0.5% to 1% by weight of tetrapolymer relative to the total weight of the composition.

### Galenics

As stated previously, the compositions according to the invention preferably comprise a physiologically acceptable medium, i.e. a non-toxic medium that may be applied to human keratin materials, which is of pleasant appearance, odour and feel.

The compositions according to the invention as defined previously and according to the chosen embodiment (choice of ingredient promoting the solubilization and/or stabilization of the phenanthrenol compounds) may be in any galenical form conventionally used for topical application and especially in the form of aqueous or aqueous-alcoholic solutions, oil-in-water (O/W) emulsions, water-in-oil (W/O) emulsions or multiple emulsions (triple: W/O/W or O/W/O), aqueous gels, or dispersions of a fatty phase in an aqueous phase with the aid of spherules, these spherules possibly being polymer nanoparticles such as nanospheres and nanocapsules, or lipid vesicles of ionic and/or nonionic type (liposomes, niosomes or oleosomes), nanoemulsions or thin films. A composition according to the invention in the form of a water and oil biphase may also be used.

According to one particular mode, it will be an aqueous gel.

According to another particular mode, it will be an emulsion.

According to yet another mode, it will be a lotion.

These compositions are prepared according to the usual methods.

In addition, the compositions used according to the invention may be more or less fluid and may have the appearance of a white or coloured cream, an ointment, a milk, a lotion, a serum, a paste or a mousse. They may optionally be applied to the skin in aerosol form. They may also be in solid form, for example in stick form.

When the composition used according to the invention comprises an oily phase, it preferably contains at least one oil. It may also contain other fatty substances.

As oils that may be used in the composition of the invention, examples that may be mentioned include:
hydrocarbon-based oils of animal origin, such as perhydrosqualene;
hydrocarbon-based oils of plant origin, such as liquid triglycerides of fatty acids containing from 4 to 10 carbon atoms, for instance heptanoic or octanoic acid triglycerides, or alternatively, for example, sunflower oil, corn oil, soybean oil, marrow oil, grapeseed oil, sesame seed oil, hazelnut oil, apricot oil, macadamia oil, arara oil, castor oil, avocado oil, caprylic/capric acid triglycerides, for instance those sold by the company Stearineries Dubois or those sold under the names Miglyol 810, 812 and 818 by the company Dynamit Nobel, jojoba oil and shea butter oil;
synthetic esters and ethers, especially of fatty acids, for instance the oils of formulae R₁COOR₂ and R₁OR₂ in which R₁ represents a fatty acid residue containing from 8 to 29 carbon atoms and R₂ represents a branched or unbranched hydrocarbon-based chain containing from 3 to 30 carbon atoms, for instance Purcellin oil, isononyl isononanoate, isopropyl myristate, 2-ethylhexyl palmitate, 2-octyldodecyl stearate, 2-octyldodecyl erucate or isostearyl isostearate; hydroxylated esters, for instance isostearyl lactate, octyl hydroxystearate, octyldodecyl hydroxystearate, diisostearyl malate or triisocetyl citrate; fatty alcohol heptanoates, octanoates or decanoates; polyol esters, for instance propylene glycol dioctanoate, neopentyl glycol diheptanoate and diethylene glycol diisononanoate; and pentaerythritol esters, for instance pentaerythrityl tetraisostearate;
linear or branched hydrocarbons, of mineral or synthetic origin, such as volatile or non-volatile liquid paraffins, and derivatives thereof, petroleum jelly, polydecenes, and hydrogenated polyisobutene such as Parleam oil;
fatty alcohols containing from 8 to 26 carbon atoms, for instance cetyl alcohol, stearyl alcohol and a mixture thereof (cetylstearyl alcohol), octyldodecanol, 2-butyloctanol, 2-hexyldecanol, 2-undecylpentadecanol, oleyl alcohol or linoleyl alcohol;
fluoro oils that are partially hydrocarbon-based and/or silicone-based, for instance those described in document JP-A-2 295 912;
silicone oils, for instance volatile or non-volatile polymethylsiloxanes (PDMS) with a linear or cyclic silicone chain, which are liquid or pasty at room temperature, especially cyclopolydimethylsiloxanes (cyclomethicones) such as cyclohexasiloxane; polydimethylsiloxanes comprising alkyl, alkoxy or phenyl groups, which are pendent or at the end of a silicone chain, these groups containing from 2 to 24 carbon atoms; phenylsilicones, for instance phenyl trimethicones, phenyl dimethicones, phenyltrimethylsiloxydiphenylsiloxanes, diphenyl dimethicones, diphenylmethyldiphenyltrisiloxanes or 2-phenylethyl trimethylsiloxy silicates, and polymethylphenylsiloxanes;
mixtures thereof.

In the list of oils mentioned above, the term “hydrocarbon-based oil” means any oil mainly comprising carbon and hydrogen atoms, and possibly ester, ether, fluoro, carboxylic acid and/or alcohol groups.

The other fatty substances that may be present in the oily phase are, for example, fatty acids containing from 8 to 30 carbon atoms, for instance stearic acid, lauric acid, palmitic acid and oleic acid; waxes, for instance lanolin wax, beeswax, carnauba wax or candelilla wax, paraffin wax, lignite wax or microcrystalline waxes, ceresin or ozokerite, and synthetic waxes, for instance polyethylene waxes and Fischer-Tropsch waxes; silicone resins such as trifluoromethyl-C1-4-alkyl dimethicone and trifluoropropyl dimethicone; and silicone elastomers, for instance the products sold under the name KSG by the company Shin-Etsu, under the name Trefil, BY29 or EPSX by the company Dow Corning, or under the name Gransil by the company Grant Industries.

These fatty substances may be chosen in a varied manner by a person skilled in the art so as to prepare a composition having the desired properties, for example in terms of consistency or texture.

According to one particular embodiment of the invention, the composition for the use according to the invention is a water-in-oil (W/O) or oil-in-water (O/W) emulsion. The proportion of the oily phase of the emulsion may range from 5% to 80% by weight and preferably from 5% to 50% by weight relative to the total weight of the composition.

The emulsions generally contain at least one emulsifier chosen from amphoteric, anionic, cationic and nonionic emulsifiers, used alone or as a mixture, and optionally a co-emulsifier. The emulsifiers are chosen in an appropriate manner according to the emulsion to be obtained (W/O or O/W emulsion). The emulsifier and the co-emulsifier are generally present in the composition in a proportion ranging for example from 0.3% to 30% by weight and preferably from 0.5% to 20% by weight relative to the total weight of the composition.

For the W/O emulsions, examples of emulsifiers that may be mentioned include dimethicone copolyols such as the mixture of cyclomethicone and of dimethicone copolyol sold under the name DC 5225 C by the company Dow Corning, and alkyl dimethicone copolyols such as the laurylmethicone copolyol sold under the name Dow Corning 5200 Formulation Aid by the company Dow Corning and the cetyl dimethicone copolyol sold under the name Abil EM 90® by the company Goldschmidt.

Surfactants for W/O emulsions that may also be used include a crosslinked elastomeric solid organopolysiloxane comprising at least one oxyalkylene group, such as those obtained according to the procedure of Examples 3, 4 and 8 of document U.S. Pat. No. 5,412,004 and of the examples of document U.S. Pat. No. 5,811,487, especially the product of Example 3 (synthesis example) of patent U.S. Pat. No. 5,412,004 and such as that sold under the reference KSG 21 by the company Shin-Etsu. Other types of KSG sold by the company Shin-Etsu may also be used, such as KSG-16.

For the O/W emulsions, examples of emulsifiers that may be mentioned include nonionic emulsifiers such as oxyalkylenated (more particularly polyoxyethylenated) fatty acid esters of glycerol; oxyalkylenated fatty acid esters of sorbitan; oxyalkylenated (oxyethylenated and/or oxypropylenated) fatty acid esters; oxyalkylenated (oxyethylenated and/or oxypropylenated) fatty alkyl ethers; sugar esters, for instance sucrose stearate; and mixtures thereof such as the mixture of glyceryl stearate and of PEG-40 stearate.

In a known manner, the cosmetic or dermatological composition of the invention may also contain adjuvants that are common in cosmetics or dermatology, such as gelling agents, film-forming polymers, preserving agents, solvents, fragrances, fillers, UV-screening agents, bactericides, odour absorbers, dyestuffs, plant extracts, salts, antioxidants, basic agents, acids, nonionic, anionic or cationic surfactants, nacres and particles.

The amounts of these various adjuvants are those conventionally used in the field under consideration, for example from 0.01% to 20% of the total weight of the composition. Depending on their nature, these adjuvants may be introduced into the fatty phase, into the aqueous phase and/or into the lipid vesicles.

As fillers that may be used in the composition of the invention, examples that may be mentioned, besides pigments, include silica powder, a colloidal amorphous silica; talc; kaolin; polyamide particles and especially those sold under the name Orgasol by the company Atochem; polyethylene powders; acrylic copolymer-based microspheres, such as those made of ethylene glycol dimethacrylate/lauryl methacrylate copolymer sold by the company Dow Corning under the name Polytrap; expanded powders such as hollow microspheres, and especially the microspheres sold under the name Expancel by the company Kemanord Plast or under the name Micropearl F 80 ED by the company Matsumoto; silicone resin microbeads such as those sold under the name Tospearl by the company Toshiba Silicone; and mixtures thereof. These fillers may be present in amounts ranging for example from 0.1% to 30% by weight and preferably from 1% to 25% by weight relative to the total weight of the composition.

Hydrophilic or lipophilic gelling agents that may especially be mentioned include carbopols, Luvigels, Hostacerin AMPS, Simulgel, acrylamide gelling agents of Sepigel type such as Sepigel 305® from SEPPIC, xanthan gum, guar gum, cellulose gum, alginates and carrageenans, and mixtures thereof. Mention may also be made of hectorites.

The surfactants that will preferably be used are foaming surfactants.

Foaming surfactants are detergents and differ from emulsifying surfactants by their HLB (hydrophilic lipophilic balance), the HLB being the ratio between the hydrophilic part and the lipophilic part in the molecule. The term HLB is well known to those skilled in the art and is described, for example, in “The HLB system. A time-saving guide to Emulsifier Selection” (published by ICI Americas Inc.; 1984).

For emulsifying surfactants, the HLB generally ranges from 3 to 8 for the preparation of W/O emulsions and from 8 to 18 for the preparation of O/W emulsions, whereas foaming surfactants generally have an HLB of greater than 20.

Advantageously, the compositions according to the invention may comprise a surfactant with an HLB of greater than 20.

The surfactant may be present in a composition according to the invention in an amount ranging for example from 0.1% to 50% by weight, preferably from 0.5% to 35% by weight, preferentially ranging for example from 0.5% to 20% by weight, in particular from 1% to 15% by weight or even from 5% to 10% by weight relative to the total weight of the composition.

a) The nonionic surfactants may be chosen, for example, from alkyl polyglucosides (APG), maltose esters, polyglycerolated fatty alcohols, and glucamine derivatives, for instance 2-ethylhexyloxycarbonyl-N-methylglucamine, and mixtures thereof. Alkylpolyglucosides that are preferably used include those containing an alkyl group containing from 6 to 30 carbon atoms and preferably from 8 to 16 carbon atoms, and containing a hydrophilic group (glucoside) preferably comprising 1.2 to 3 glucoside units. Examples of alkylpolyglucosides that may be mentioned include decylglucoside (Alkyl-C9/C11-polyglucoside (1.4)) for instance the product sold under the name Mydol 10® by the company Kao Chemicals, the product sold under the name Plantaren 2000 UP® by the company Cognis, and the product sold under the name Oramix NS 10® by the company SEPPIC; caprylyl/capryl glucoside, for instance the product sold under the name Oramix CG 110® by the company SEPPIC; lauryl glucoside, for instance the products sold under the names Plantaren 1200 N® and Plantacare 1200® by the company Cognis; cocoglucoside, for instance the product sold under the name Plantacare 818/UP® by the company Cognis; and mixtures thereof.

The maltose derivatives are, for example, those described in document EP-A-566 438, such as O-octanoyl-6′-D-maltose or O-dodecanoyl-6′-D-maltose described in document FR-2 739 556.

Among the polyglycerolated fatty alcohols that may be mentioned is polyglycerolated dodecanediol (3.5 mol of glycerol), this product being sold under the name Chimexane NF® by the company Chimex.

b) The anionic surfactants may be chosen, for example, from soaps (alkali metal salts of fatty acids), carboxylates, acylamino acids, amidoether carboxylates, alkyl polyaminocarboxylates, alkyl ether sulfates such as sodium laureth sulfates, alkyl sulfonates, isethionates, alkyl methyltaurate, alkyl sulfosuccinates, alkyl sulfoacetates and alkyl phosphates (monoalkyl or dialkyl phosphates), salts thereof, and mixtures thereof.

Carboxylates that may especially be mentioned include alkyl glycol carboxylic acids (or 2-(2-hydroxyalkyloxyacetic acids)) and salts thereof, for instance sodium lauryl glycol carboxylate, sold under the names Beaulight Shaa® or Beaulight LCA-25N® by the company Sanyo (CTFA name: sodium lauryl glycol carboxylate), or its corresponding acid form sold under the name Beaulight Shaa (Acid Form)® by the company Sanyo.

Examples of acylamino acids that may be mentioned include sodium cocoylglycinate sold by the company Ajinomoto under the name Amilite GCS 12, sodium lauroyl glutamate sold by the company Ajinomoto under the name Amisoft LS11 and sodium lauroyl sarcosinate sold by the company SEPPIC under the name Oramix L30.

An example of an alkyl phosphate that may be mentioned is lauryl phosphate, sold by the company Kao under the name MAP 20.

c) The amphoteric and zwitterionic foaming surfactants may be chosen, for example, from betaine derivatives including amidopropylbetaines, amphoacetates and amphodiacetates, and hydroxylsultaines, and mixtures thereof.

Examples of betaine derivatives that may be mentioned include cocobetaine, for instance the product sold under the name Dehyton AB-30® by the company Cognis; laurylbetaine, for instance the product sold under the name Genagen KB® by the company Clariant; oxyethylenated (10 EO) laurylbetaine, for instance the product sold under the name Lauryl ether (10 OE) Betaine® by the company Shin Nihon Rica; oxyethylenated (10 EO) stearylbetaine, for instance the product sold under the name Stearyl ether (10 OE) Betaine® by the company Shin Nihon Rica; the cocamidopropyl betaine sold, for example, under the name Velvetex BK 35® by the company Cognis; the undecylenamidopropyl betaine sold, for example, under the name Amphoram U® by the company Ceca; and mixtures thereof.

Examples of amphoacetates and amphodiacetates that may be mentioned include N-disodium N-cocoyl-N-carboxymethoxyethyl-N-carboxymethylethylenediamine (CTFA name: disodium cocamphodiacetate), for instance the product sold under the name Miranol C2M Concentrate NP® by the company Rhodia Chimie; N-sodium N-cocoyl-N-hydroxyethyl-N-carboxymethylethylenediamine (CTFA name: sodium cocamphoacetate), and mixtures thereof.

According to one embodiment of the invention, a surfactant that is suitable for use in the invention may be chosen from alkylpolyglucosides, betaine derivatives, alkyl glycol carboxylic acids and salts thereof, alkyl ether sulfates, alkyl phosphates, amphodiacetates, amphoacetates, alkylglycinates, acylglutamates and acyl sarcosinates, and mixtures thereof.

According to one embodiment, a surfactant that is suitable for use in the invention may be chosen from decyl glucoside, cocoyl glucoside, sodium lauryl ether sulfate, cocoylbetaine, lauroylbetaine, cocoamidopropylbetaine, lauramidopropylbetaine, lauryl glycol carboxylate, cocoampho(di)acetate, lauroampho(di)acetate and potassium lauryl phosphate, or a mixture thereof.

According to one particular mode, the composition contains at least one foaming surfactant chosen from decyl glucoside, sodium lauryl ether sulfate and PEG-7 glyceryl cocoate, and mixtures thereof.

According to one particular mode, when the composition contains a foaming surfactant, it does not contain any oxyalkylenated compound.

The use of a phenanthrenol compound according to the invention in combination with at least one foaming surfactant improves the foam quality of the compositions containing at least one foaming surfactant.

This combination is especially advantageous in cleansing and/or makeup-removing compositions.

Preferred foaming surfactants that may be mentioned include:
sodium cocoyl isethionate, such as the sodium cocoyl isethionate/sodium isethionate mixture sold by BASF under the name Jordapon CI P®;
decyl glucoside, such as Plantacare 2000 UP® sold by Cognis;
potassium laurate, such as Potassium Laurate DUB LK® sold by the company Stearinerie Dubois;
sodium lauroyl oat amino acids, such as the aqueous 30% solution of lauroyl oat amino acids, in the form of the protected sodium salt (1.4% phenonip) sold under the name Proteol Oat® by SEPPIC;
potassium lauroyl wheat amino acids, such as hydrolysed potassium lauroyl wheat protein at 25% in water, and stabilized (0.4-0.4% phenonip-potassium sorbate), sold under the name Aminofoam W OR® by Croda;
polyglyceryl-3 hydroxylauryl ether or polyglycerolated dodecanediol (3.5 mol of glycerol), such as Chimexane® from Chimex;
sodium lauryl ether sulfate, such as Texapon AOS 225 UP® from Cognis;
N-disodium N-cocoyl-N-carboxymethoxyethyl-N-carboxymethylethylenediamine (CTFA name: disodium cocoamphodiacetate), for instance the product sold under the name Miranol C2M Concentrate NP® by the company Rhodia Chimie;
cocobetaine or cocoylbetaine in aqueous solution, such as Mirataine BB/FLA from Rhodia;
PEG-7 glyceryl cocoate or oxyethylenated (7 EO) glyceryl cocoate, such as Cetiol HE® from Cognis;
lauroylbetaine such as lauryl dimethylamine betaine as an aqueous 30% solution, sold under the name Empigen BB/LS® by the company Huntsman;
sodium cocoamphoacetate, such as sodium N-cocoyl amidoethyl N-hydroxyethyl glycinate as an aqueous 32% solution sold under the name Miranol Ultra C 32® by Rhodia;
triethanolamine lauryl sulfate or TEA lauryl sulfate, such as triethanolamine lauryl sulfate (70/30 C12/C14) as an aqueous 40% solution sold under the name Texapon T42® by Cognis;
sodium lauryl glycol carboxylate, sold under the names Beaulight SHAA® or Beaulight LCA-25N® by the company Sanyo (CTFA name: sodium lauryl glycol carboxylate), or its corresponding acid form sold under the name Beaulight SHAA (Acid Form)® by the company Sanyo;
lauryl phosphate sold by the company Kao under the name MAP 20;
sodium cocoyl glycinate, such as Amilite GCS® 12 from Ajinomoto.

According to one particular mode, the composition according to the invention comprises at least one phenanthrenol compound and at least sodium cocoyl isethionate, such as the sodium cocoyl isethionate/sodium isethionate mixture sold by BASF under the name Jordapon CI P®.

According to another mode, the composition according to the invention comprises at least one phenanthrenol compound and at least decyl glucoside, such as Plantacare 2000 UP® sold by Cognis, the composition not comprising any oxyalkylenated compound.

According to another mode, the composition according to the invention comprises at least one phenanthrenol compound and at least potassium laurate, such as Potassium Laurate DUB LK® sold by the company Stearinerie Dubois.

According to another mode, the composition according to the invention comprises at least one phenanthrenol compound and at least sodium lauroyl oat amino acids, such as the aqueous 30% solution of lauroyl oat amino acids, in the form of the protected sodium salt (1.4% phenonip) sold under the name Proteol Oat® by SEPPIC.

According to one particular mode, the composition according to the invention comprises at least one phenanthrenol compound and at least potassium lauroyl wheat amino acids, such as hydrolysed potassium lauroyl wheat protein at 25% in water, and stabilized (0.4-0.4% phenonip-potassium sorbate), sold under the name Aminofoam W OR® by Croda.

According to another mode, the composition according to the invention comprises at least one phenanthrenol compound and at least polyglyceryl-3 hydroxylauryl ether or polyglycerolated dodecanediol (3.5 mol of glycerol), such as Chimexane® from Chimex, the composition not comprising any oxyalkylenated compound. According to one particular mode, the composition according to the invention comprises at least one phenanthrenol compound and at least sodium lauryl ether sulfate, such as Texapon AOS 225 OR® from Cognis, the composition not comprising any oxyalkylenated compound.

According to another mode, the composition according to the invention comprises at least one phenanthrenol compound and at least N-disodium N-cocoyl-N-carboxymethoxyethyl-N-carboxymethylethylenediamine (CTFA name: disodium cocamphodiacetate), for instance the product sold under the name Miranol C2M Concentrate NP® by the company Rhodia Chimie, the composition not comprising any oxyalkylenated compound.

According to another mode, the composition according to the invention comprises at least one phenanthrenol compound and at least cocobetaine or cocoylbetaine as an aqueous solution, such as Mirataine BB/FLA from Rhodia; the composition not comprising any oxyalkylenated compound.

According to another mode, the composition according to the invention comprises at least one phenanthrenol compound and at least PEG-7 glyceryl cocoate or oxyethylenated (7 EO) glyceryl cocoate, such as Cetiol HE® from Cognis.

According to one particular mode, the composition according to the invention comprises at least one phenanthrenol compound and at least lauryl betaine such as lauryl dimethylamine betaine as an aqueous 30% solution, sold under the name Empigen BB/LS® by the company Huntsman, the composition not comprising any oxyalkylenated compound.

According to another mode, the composition according to the invention comprises at least one phenanthrenol compound and at least sodium cocoamphoacetate, such as sodium N-cocoyl amidoethyl N-hydroxyethyl glycinate as an aqueous 32% solution sold under the name Miranol Ultra C 32® by Rhodia, the composition not comprising any oxyalkylenated compound.

According to another mode, the composition according to the invention comprises at least one phenanthrenol compound and at least triethanolamine lauryl sulfate or TEA lauryl sulfate, such as the triethanolamine lauryl sulfate (70/30 C12/C14) as an aqueous 40% solution sold under the name Texapon T 42® by Cognis.

According to another mode, the composition according to the invention comprises at least one phenanthrenol compound and at least sodium lauryl glycol carboxylate, sold under the names Beaulight SHAA® or Beaulight LCA-25N® by the company Sanyo (CTFA name: Sodium Lauryl Glycol Carboxylate), or its corresponding acid form sold under the name Beaulight SHAA (Acid Form)® by the company Sanyo, the composition not comprising any oxyalkylenated compound.

According to one particular mode, the composition according to the invention comprises at least one phenanthrenol compound and at least lauryl phosphate sold by the company Kao under the name a MAP 20®, the composition not comprising any oxyalkylenated compound.

According to another mode, the composition according to the invention comprises at least one phenanthrenol compound and at least sodium cocoyl glycinate, such as Amilite GCS® 12 from Ajinomoto, the composition not comprising any oxyalkylenated compound.

The use of a phenanthrenol compound according to the invention in combination with at least one filler improves the dispersing effect of the fillers in the compositions containing at least one filler.

Preferred fillers that may especially be mentioned include:
hollow hemispherical particles of silicone resins, for instance NLK 500®, NLK 506® and NLK 510® from Takemoto Oil and Fat, described especially in EP-A-1 579 849;
polyamide fibres, such as Nylon® fibres, in particular Nylon-66 sold under the name Pulpe Polyamide 12185 by Utexbel or Fiberlon 931-D1-S by LCW;
microencapsulated iron oxides, in particular:
   microencapsulated yellow iron oxides, such as the mixture of iron oxides, of titanium oxides, of boron nitride, of acrylates and ammonium methacrylate copolymer, and of triethyl citrate, sold under the name Yellowcap1® by the company Tagra Biotechnologies Ltd;
   microencapsulated red iron oxides, such as the mixture of iron oxides, of titanium oxides, of boron nitride, of acrylates and ammonium methacrylate copolymer, and of triethyl citrate, sold under the name Redcap1® by the company Tagra Biotechnologies Ltd;
   microencapsulated black iron oxides, such as the mixture of iron oxides, of titanium oxides, of boron nitride, of acrylates and ammonium methacrylate copolymer, and of triethyl citrate, sold under the name Blackcap1® by the company Tagra Biotechnologies Ltd.

According to one particular mode, the composition according to the invention contains at least one phenanthrenol compound and at least hollow hemispherical particles of silicone resins, for instance NLK 500®, NLK 506® and NLK 510® from Takemoto Oil and Fat, described especially in EP-A-1 579 849.

According to another mode, the composition according to the invention contains at least one phenanthrenol compound and at least polyamide fibres, such as Nylon® fibres, in particular Nylon-66 sold under the name Pulpe Polyamide 12185 by Utexbel or Fiberlon 931-D1-S by LCW.

According to another mode, the composition according to the invention contains at least one phenanthrenol compound and at least microencapsulated iron oxides, in particular:
microencapsulated yellow iron oxides, such as the mixture of iron oxides, of titanium oxides, of boron nitride, of acrylates and ammonium methacrylate copolymer, and of triethyl citrate, sold under the name Yellowcap1® by the company Tagra Biotechnologies Ltd;
microencapsulated red iron oxides, such as the mixture of iron oxides, of titanium oxides, of boron nitride, of acrylates and ammonium methacrylate copolymer, and of triethyl citrate, sold under the name Redcap1® by the company Tagra Biotechnologies Ltd;
microencapsulated black iron oxides, such as the mixture of iron oxides, of titanium oxides, of boron nitride, of acrylates and ammonium methacrylate copolymer, and of triethyl citrate, sold under the name Blackcap1® by the company Tagra Biotechnologies Ltd.

The combination between a phenanthrenol compound and fillers will be especially advantageous in skincare and/or skin makeup compositions.

Antioxidants that may especially be mentioned include polyphenols, tannic acid, epigallocatechins and natural extracts containing them, anthocyans, extracts of rosemary, extracts of olive leaves, green tea, resveratrol and derivatives thereof, pycnogenol, ergothioneine, N-acetylcysteine, biotin, chelating agents, idebenone, plant extracts, for instance Pronalen Bioprotect™ from the company Provital, free-radical scavengers, for instance vitamin E, coenzyme Q10, bioflavonoids, SOD, phytanetriol, lignans, melatonin, pidolates and glutathione.

According to one preferred embodiment of the invention, the compound used according to the invention contains at least one photoprotective agent, which may be organic or mineral.

The organic photoprotective agents are chosen especially from anthranilates; cinnamic derivatives; salicylic derivatives; camphor derivatives; benzophenone derivatives; β,β-diphenylacrylate derivatives; triazine derivatives; benzotriazole derivatives; benzalmalonate derivatives, especially those cited in U.S. Pat. No. 5,624,663; benzimidazole derivatives; imidazolines; bis-benzazolyl derivatives as described in patents EP 669 323 and U.S. Pat. No. 2,463,264; p-aminobenzoic acid (PABA) derivatives; methylenebis(hydroxyphenylbenzotriazole) derivatives as described in patent applications U.S. Pat. No. 5,237,071, U.S. Pat. No. 5,166,355, GB 2 303 549, DE 197 26 184 and EP 893 119; benzoxazole derivatives as described in patent applications EP 0 832 642, EP 1 027 883, EP 1 300 137 and DE 101 62 844; screening polymers and screening silicones such as those described especially in patent application WO 93/04665; dimers derived from α-alkylstyrene, such as those described in patent application DE 198 55 649; 4,4-diarylbutadienes such as those described in patent applications EP 0 967 200, DE 197 46 654, DE 197 55 649, EP-A-1 008 586, EP 1 133 980 and EP 133 981, and mixtures thereof.

As examples of additional organic photoprotective agents, mention may be made of those denoted hereinbelow under their INCI name:

para-Aminobenzoic Acid Derivatives:

### PABA,

### Ethyl PABA,

Ethyl dihydroxypropyl PABA,
Ethylhexyl dimethyl PABA sold in particular under the name Escalol 507 by ISP,

### Glyceryl PABA,

PEG-25 PABA sold under the name Uvinul P25 by BASF.

### Salicylic Derivatives:

Homosalate sold under the name Eusolex HMS by Rona/EM Industries,
Ethylhexyl salicylate sold under the name Neo Heliopan OS by Haarmann and Reimer,
Dipropylene glycol salicylate sold under the name Dipsal by Scher,
TEA salicylate sold under the name Neo Heliopan TS by Haarmann and Reimer.

### Cinnamic Derivatives:

Ethylhexyl methoxycinnamate sold in particular under the trade name Parsol MCX by Hoffmann LaRoche,
Isopropyl methoxycinnamate,
Isoamyl methoxycinnamate sold under the trade name Neo Heliopan E 1000 by Haarmann and Reimer,

### Cinoxate,

DEA methoxycinnamate,
Diisopropyl methylcinnamate,
   Glyceryl ethylhexanoate dimethoxycinnamate.

### β,β-Diphenylacrylate Derivatives:

Octocrylene sold in particular under the trade name Uvinul N539 by BASF,
Etocrylene sold in particular under the trade name Uvinul N35 by BASF.

### Benzophenone Derivatives:

Benzophenone-1 sold under the trade name Uvinul 400 by BASF,
   Benzophenone-2 sold under the trade name Uvinul D50 by BASF,
   Benzophenone-3 or Oxybenzone sold under the trade name Uvinul M40 by BASF,
   Benzophenone-4 sold under the trade name Uvinul MS40 by BASF,

### Benzophenone-5,

Benzophenone-6 sold under the trade name Helisorb 11 by Norquay,
Benzophenone-8 sold under the trade name Spectra-Sorb UV-24 by American Cyanamid,
Benzophenone-9 sold under the trade name Uvinul DS-49 by BASF,

### Benzophenone-12

n-hexyl 2-(4-diethylamino-2-hydroxybenzoyl)benzoate sold under the trade name Uvinul A+ or as a mixture with octyl methoxycinnamate under the trade name Uvinul A+B by the company BASF.

### Benzylidenecamphor Derivatives:

3-Benzylidenecamphor manufactured under the name Mexoryl SD by Chimex,
4-Methylbenzylidenecamphor sold under the name Eusolex 6300 by Merck,
Benzylidenecamphorsulfonic acid manufactured under the name Mexoryl SL by Chimex,
Camphor benzalkonium methosulfate manufactured under the name Mexoryl SO by Chimex,
Terephthalylidenedicamphorsulfonic acid manufactured under the name Mexoryl SX by Chimex,
Polyacrylamidomethylbenzylidenecamphor manufactured under the name Mexoryl SW by Chimex.

### Phenylbenzimidazole Derivatives:

Phenylbenzimidazolesulfonic acid sold in particular under the trade name Eusolex 232 by Merck,
Disodium phenyldibenzimidazoletetrasulfonate sold under the trade name Neo Heliopan AP by Haarmann and Reimer.

### Phenylbenzotriazole Derivatives:

Drometrizole trisiloxane sold under the name Silatrizole by Rhodia Chimie,
Methylenebis(benzotriazolyl)tetramethylbutylphenol sold in solid form under the trade name MIXXIM BB/100 by Fairmount Chemical, or in micronized form as an aqueous dispersion under the trade name Tinosorb M by Ciba Specialty Chemicals.

### Triazine Derivatives:

bis-Ethylhexyloxyphenol methoxyphenyl triazine sold under the trade name Tinosorb S by Ciba Geigy,
Ethylhexyl triazone sold in particular under the trade name Uvinul T150 by BASF,
Diethylhexyl butamido triazone sold under the trade name Uvasorb HEB by Sigma 3V,
2,4,6-tris(Dineopentyl 4′-aminobenzalmalonate)-s-triazine,
2,4,6-tris(Diisobutyl 4′-aminobenzalmalonate)-s-triazine,
the symmetrical triazine screening agents described in U.S. Pat. No. 6,225,467, patent application WO 2004/085 412 (see compounds 6 and 9) or the document “Symmetrical Triazine Derivatives” IP.COM Journal, IP.COM INC WEST HENRIETTA, NY, US (20 Sep. 2004), especially 2,4,6-tris(biphenyl)-1,3,5-triazines (in particular 2,4,6-tris(biphenyl-4-yl-1,3,5-triazine) and 2,4,6-tris(terphenyl)-1,3,5-triazine which is also mentioned in patent applications WO 06/035 000, WO 06/034 982, WO 06/034 991, WO 06/035 007, WO 2006/034 992 and WO 2006/034 985.

### Anthranilic Derivatives:

Menthyl anthranilate sold under the trade name Neo Heliopan MA by Haarmann and Reimer.

### Imidazoline Derivatives:

Ethylhexyldimethoxybenzylidenedioxoimidazoline propionate.

### Benzalmalonate Derivatives:

Dineopentyl 4′-methoxybenzalmalonate,
Polyorganosiloxane containing benzalmalonate functions, for instance Polysilicone-15, sold under the trade name Parsol SLX by Hoffmann LaRoche

### 4,4-Diarylbutadiene Derivatives:

1,1-Dicarboxy(2,2′-dimethylpropyl)-4,4-diphenylbutadiene,

### Benzoxazole Derivatives:

2,4-bis[5-1(dimethylpropyl)benzoxazol-2-yl(4-phenyl)imino]-6-(2-ethylhexyl)imino-1,3,5-triazine sold under the name Uvasorb K2A by Sigma 3V and mixtures thereof.

The preferential additional organic photoprotective agents are chosen from:

Ethylhexyl methoxycinnamate,

### Homosalate,

Ethylhexyl salicylate,

### Octocrylene,

Phenylbenzimidazolesulfonic acid,

### Benzophenone-3,

### Benzophenone-4,

### Benzophenone-5,

n-Hexyl 2-(4-diethylamino-2-hydroxybenzoyl)benzoate,

### 4-Methylbenzylidenecamphor,

Terephthalylidenedicamphorsulfonic acid,
Disodium phenyldibenzimidazoletetrasulfonate,

### Methylenebis(benzotriazolyl)tetramethylbutylphenol,

Ethylhexyl triazone,
bis-Ethylhexyloxyphenol methoxyphenyl triazine,
Diethylhexyl butamido triazone,
2,4,6-tris(Dineopentyl 4′-aminobenzalmalonate)-s-triazine,
2,4,6-tris(Diisobutyl 4′-aminobenzalmalonate)-s-triazine,
2,4,6-tris(Biphenyl-4-yl-1,3,5-triazine),
2,4,6-tris(Terphenyl)-1,3,5-triazine
Drometrizole trisiloxane,

### Polysilicone-15,

Dineopentyl 4′-methoxybenzalmalonate,
1,1-Dicarboxy(2,2′-dimethylpropyl)-4,4-diphenylbutadiene,
2,4-Bis[5-1(dimethylpropyl)benzoxazol-2-yl(4-phenyl)imino]-6-(2-ethylhexyl)imino-1,3,5-triazine,
and mixtures thereof.

The mineral photoprotective agents are chosen from coated or uncoated metal oxide pigments (mean size of the primary particles: generally between 5 nm and 100 nm and preferably between 10 nm and 50 nm), for instance titanium oxide (amorphous or crystallized in rutile and/or anatase form), iron oxide, zinc oxide, zirconium oxide or cerium oxide pigments, which are all UV-photoprotective agents that are well known per se. The pigments may be coated or uncoated.

The coated pigments are pigments that have undergone one or more surface treatments of chemical, electronic, mechanochemical and/or mechanical nature with compounds as described, for example, in Cosmetics & Toiletries, February 1990, Vol. 105, pp. 53-64, such as amino acids, beeswax, fatty acids, fatty alcohols, anionic surfactants, lecithins, sodium, potassium, zinc, iron or aluminium salts of fatty acids, metal alkoxides (of titanium or of aluminium), polyethylene, silicones, proteins (collagen, elastin), alkanolamines, silicon oxides, metal oxides or sodium hexametaphosphate.

As is known, silicones are organosilicon polymers or oligomers of linear or cyclic, branched or crosslinked structure, of variable molecular weight, obtained by polymerization and/or polycondensation of suitably functionalized silanes, and consist essentially of a repetition of main units in which the silicon atoms are linked together via oxygen atoms (siloxane bond), optionally substituted hydrocarbon-based radicals being directly attached via a carbon atom to the silicon atoms.

The term “silicones” also includes the silanes required for their preparation, in particular alkyl silanes.

The silicones used for coating the nanopigments that are suitable for the present invention are preferably chosen from the group containing alkyl silanes, polydialkylsiloxanes and polyalkylhydrogenosiloxanes. Even more preferentially, the silicones are chosen from the group containing octyltrimethylsilane, polydimethylsiloxanes and polymethylhydrogenosiloxanes.

Needless to say, before being treated with silicones, the metal oxide pigments may have been treated with other surface agents, in particular with cerium oxide, alumina, silica, aluminium compounds or silicon compounds, or mixtures thereof.

The coated pigments are more particularly titanium oxides that have been coated:
with silica, such as the product Sunveil from the company Ikeda and the product Eusolex T-AVO from the company Merck,
with silica and iron oxide, such as the product Sunveil F from the company Ikeda,
with silica and alumina, such as the products Microtitanium Dioxide MT 500 SA and Microtitanium Dioxide MT 100 SA from the company Tayca, Tioveil from the company Tioxide and Mirasun TiW 60 from the company Rhodia,
with alumina, such as the products Tipaque TTO-55 (B) and Tipaque TTO-55 (A) from the company Ishihara and UVT 14/4 from the company Kemira,
with alumina and aluminium stearate, such as the product Microtitanium Dioxide MT 100 TV, MT 100 TX, MT 100 Z and MT-01 from the company Tayca, and the products Solaveil CT-10 W, Solaveil CT 100 and Solaveil CT 200 from the company Uniqema,
with silica, alumina and alginic acid, such as the product MT-100 AQ from the company Tayca,
with alumina and aluminium laurate, such as the product Microtitanium Dioxide MT 100 S from the company Tayca,
with iron oxide and iron stearate, such as the product Microtitanium Dioxide MT 100 F from the company Tayca,
with zinc oxide and zinc stearate, such as the product BR351 from the company Tayca,
with silica and alumina and treated with a silicone, such as the products Microtitanium Dioxide MT 600 SAS, Microtitanium Dioxide MT 500 SAS or Microtitanium Dioxide MT 100 SAS from the company Tayca,
with silica, alumina and aluminium stearate and treated with a silicone, such as the product STT-30-DS from the company Titan Kogyo,
with silica and treated with a silicone, such as the product UV-Titan X 195 from the company Kemira, or the product SMT-100 WRS from the company Tayca,
with alumina and treated with a silicone, such as the products Tipaque TTO-55 (S) from the company Ishihara or LW Titan M 262 from the company Kemira,
with triethanolamine, such as the product STT-65-S from the company Titan Kogyo,
with stearic acid, such as the product Tipaque TTO-55 (C) from the company Ishihara,
with sodium hexametaphosphate, such as the product Microtitanium Dioxide MT 150 W from the company Tayca.

Other titanium oxide pigments treated with a silicone are preferably TiO₂ treated with octyltrimethylsilane and for which the mean size of the elementary particles is between 25 and 40 nm, such as the product sold under the trade name T 805 by the company Degussa Silices, TiO₂ treated with a polydimethylsiloxane and for which the mean size of the elementary particles is 21 nm, such as the product sold under the trade name 70250 Cardre UF TiO2SI3 by the company Cardre, anatase/rutile TiO₂ treated with a polydimethylhydrogenosiloxane and for which the mean size of the elementary particles is 25 nm, such as the product sold under the trade name Microtitanium Dioxide USP Grade Hydrophobic by the company Color Techniques.

The uncoated titanium oxide pigments are sold, for example, by the company Tayca under the trade names Microtitanium Dioxide MT 500 B or Microtitanium Dioxide MT 600 B, by the company Degussa under the name P 25, by the company Wackher under the name Transparent titanium oxide PW, by the company Myoshi Kasei under the name UFTR, by the company Tomen under the name ITS and by the company Tioxide under the name Tioveil AQ.

The uncoated zinc oxide pigments are, for example:
those sold under the name Z-Cote by the company Sunsmart;
those sold under the name Nanox by the company Elementis;
those sold under the name Nanogard WCD 2025 by the company Nanophase Technologies.

The coated zinc oxide pigments are, for example:
those sold under the name Z-Cote HP1 by the company Sunsmart (dimethicone-coated ZnO);
those sold under the name Zinc Oxide CS-5 by the company Toshibi (ZnO coated with polymethylhydrogenosiloxane);
those sold under the name Nanogard Zinc Oxide FN by the company Nanophase Technologies (as a 40% dispersion in Finsolv TN, C₁₂-C₁₅ alkyl benzoate);
those sold under the name Daitopersion ZN-30 and Daitopersion ZN-50 by the company Daito (dispersions in cyclopolymethylsiloxane/oxyethylenated polydimethylsiloxane, containing 30% or 50% of nanozinc oxides coated with silica and polymethylhydrogenosiloxane);
those sold under the name NFD Ultrafine ZnO by the company Daikin (ZnO coated with perfluoroalkyl phosphate and copolymer based on perfluoroalkylethyl as a dispersion in cyclopentasiloxane);
those sold under the name SPD-Z1 by the company Shin-Etsu (ZnO coated with silicone-grafted acrylic polymer, dispersed in cyclodimethylsiloxane);
those sold under the name Escalol Z100 by the company ISP (alumina-treated ZnO dispersed in an ethylhexyl methoxycinnamate/PVP-hexadecene/methicone copolymer mixture);
those sold under the name Fuji ZnO-SMS-10 by the company Fuji Pigment (ZnO coated with silica and polymethylsilsesquioxane);
those sold under the name Nanox Gel TN by the company Elementis (ZnO dispersed at a concentration of 55% in C₁₂-C₁₅ alkyl benzoate with hydroxystearic acid polycondensate).

The uncoated cerium oxide pigments are sold under the name Colloidal Cerium Oxide by the company Rhone-Poulenc.

The uncoated iron oxide nanopigments are sold, for example, by the company Arnaud under the names Nanogard WCD 2002 (FE 45B), Nanogard Iron FE 45 BL AQ, Nanogard FE 45R AQ and Nanogard WCD 2006 (FE 45R) or by the company Mitsubishi under the name TY-220,

The coated iron oxide pigments are sold, for example, by the company Arnaud under the names Nanogard WCD 2008 (FE 45B FN), Nanogard WCD 2009 (FE 45B 556), Nanogard FE 45 BL 345 and Nanogard FE 45 BL or by the company BASF under the name Transparent Iron Oxide.

Mention may also be made of mixtures of metal oxides, especially of titanium dioxide and of cerium dioxide, including the silica-coated equal-weight mixture of titanium dioxide and of cerium dioxide, sold by the company Ikeda under the name Sunveil A, and also the alumina, silica and silicone-coated mixture of titanium dioxide and of zinc dioxide, such as the product M 261 sold by the company Kemira, or the alumina, silica and glycerol-coated mixture of titanium dioxide and of zinc dioxide, such as the product M 211 sold by the company Kemira.

The photoprotective agents are generally present in the compositions according to the invention in proportions ranging for example from 0.01% to 20% by weight relative to the total weight of the composition, and preferably ranging for example from 0.1% to 10% by weight relative to the total weight of the composition.

The compositions according to the invention may be applied directly to the skin or, alternatively, to cosmetic or dermatological supports of occlusive or non-occlusive type, intended to be applied locally to the skin.

The support may be an “occlusive” support. By way of example, the support is constituted by a thermoplastic material, chosen from high-density and low-density polyethylenes, polypropylenes, polyvinyl chlorides, copolymers of ethylene and of vinyl acetate, polyesters and polyurethanes, or by a complex of such materials. These materials may also be present in stratified form with at least one metal foil such as an aluminium foil.

The support layer may be of any suitable thickness that affords the desired support and protective functions. Preferably, the thickness of the support layer is between about 20 μm and about 1.5 mm. Advantageously, the support layer is sufficiently flexible, so as to be able to follow the profile of the skin perfectly, and so as not to cause the user any sensation of discomfort.

Preferably, however, the support is “non-occlusive”. In the latter hypothesis, a support constituted by a paper, a porous or perforated thermoplastic material, a fabric, a nonwoven or a perforated nonwoven is advantageously used.

The phenanthrenol compound according to the invention may be used according to the invention as antioxidant.

The phenanthrenol compound per se and/or the composition containing it may be used according to the invention for improving the appearance and/or visibility of the pores, in particular by reducing the size and/or number of visible pores on facial skin, and especially in the T area (forehead, nose, cheeks and chin), in particular in the region of the nose and the cheeks.

According to one particular mode, the composition according to the invention will also comprise at least one other active agent chosen from an extract of black tea, an extract of brown sugar and an extract of lychee, and mixtures thereof. In particular, the combination and/or the composition containing it is used according to the invention for improving the tightening of the pores of the skin, in particular on the face and especially on the T area (forehead, nose, cheeks and chin), in particular in the region of the nose and the cheeks.

The phenanthrenol compound per se and/or the composition containing it may be used according to the invention for reducing the imperfections of greasy skin, in particular on the face, and especially in the T area (forehead, nose, cheeks and chin).

According to one particular mode, the composition according to the invention will also comprise at least one other active agent chosen from desquamating agents, sebo-regulating or anti-seborrhoeic agents, and calmatives, and a mixture thereof. The composition may also comprise at least one ingredient such as fillers with a soft-focus effect or matting agents, for providing an immediate visual effect.

According to one preferred mode, a composition for treating the imperfections of greasy skin according to the invention will comprise at least one phenanthrenol compound, at least one desquamating agent, at least one calmative, and optionally at least one ingredient for providing an immediate visual effect.

Preferably, the desquamating agent will be an n-octanoylsalicylic acid, optionally in combination with salicylic acid.

Preferably, the calmative will be chosen from a glycyrrhetinic acid salt, in particular a potassium salt of glycyrrhetinic acid, an extract of Eperua falcata bark such as the product sold by the company Cognis under the name Eperuline®, and mixtures thereof.

The phenanthrenol compound per se and/or the composition containing it may be used according to the invention for preventing and/or treating ageing in the area around the lips, in particular the moustache area.

According to one particular mode, the phenanthrenol compound may be used in combination with at least one agent chosen from cinnamic acid, phloroglucinol and an extract of soybean, and mixtures thereof. Preferably, the phenanthrenol compound is combined with a phytocomplex comprising cinnamic acid, phloroglucinol and an extract of soybean. This combination will be especially advantageous for preventing and/or treating hormonal ageing in the area around the lips, in particular the moustache area.

According to another embodiment of the invention, the compositions according to the invention may be combined with orally administered compositions containing additional cosmetic active agents that have a beneficial effect on the appearance of the skin, for instance additional active agents for combating the signs of ageing of the skin or additional active agents for combating greasy skin.

The additional ingredients used in the composition of the invention may also be a cosmetic or pharmaceutical ingredient and/or active agent, in particular ingredients and/or active agents that are beneficial to the appearance and/or texture of the skin.

According to one particular mode of the invention, the composition according to the invention comprises at least one ingredient for solubilizing and/or stabilizing the phenanthrenol compound and also at least one other additional ingredient and/or active agent chosen from a cosmetic or pharmaceutical ingredient and/or active agent.

### C—Additional Cosmetic and Dermatological Active Agents:

The additional active agents may be chosen especially from moisturizers, desquamating agents, agents for improving the barrier function, depigmenting agents, antioxidants, dermo-decontracting agents, anti-glycation agents, agents for stimulating the synthesis of dermal and/or epidermal macromolecules and/or for preventing their degradation, agents for stimulating fibroblast or keratinocyte proliferation and/or keratinocyte differentiation, agents for promoting the maturation of the horny envelope, NO-synthase inhibitors, peripheral benzodiazepine receptor (PBR) antagonists, agents for increasing the activity of the sebaceous glands, agents for stimulating the energy metabolism of cells, tensioning agents, lipo-restructuring agents, slimming agents, agents for promoting cutaneous microcirculation, calmatives and/or anti-irritants, sebo-regulating or anti-seborrhoeic agents, astringents, cicatrizing agents, anti-inflammatory agents and anti-acne agents.

According to one particular mode, the additional active agents may be chosen from desquamating agents, depigmenting agents, antioxidants, anti-glycation agents, agents for stimulating the synthesis of dermal and/or epidermal macromolecules and/or for preventing their degradation, calmatives and/or anti-irritant agents, sebo-regulating or anti-seborrhoeic agents, and astringents.

A person skilled in the art will choose the active agent(s) as a function of the effect desired on the keratin materials.

For caring for and/or making up aged skin, he will preferably choose at least one active agent chosen from moisturizers, desquamating agents, agents for improving the barrier function, depigmenting agents, antioxidants, dermo-decontracting agents, anti-glycation agents, agents for stimulating the synthesis of dermal and/or epidermal macromolecules and/or for preventing their degradation, agents for stimulating fibroblast or keratinocyte proliferation and/or keratinocyte differentiation, agents for promoting the maturation of the horny envelope, NO-synthase inhibitors, peripheral benzodiazepine receptor (PBR) antagonists, agents for increasing the activity of the sebaceous glands, agents for stimulating the energy metabolism of cells, and agents for promoting the cutaneous microcirculation for the area around the eyes.

The composition may also comprise at least one ingredient such as fillers with a soft-focus effect or agents for promoting the natural coloration of the skin, intended for complementing the biological effects of these active agents or for providing an immediate visual anti-ageing effect.

For caring for, cleansing and/or making up greasy skin, a person skilled in the art will preferably choose at least one active agent chosen from desquamating agents, sebo-regulating agents or anti-seborrhoeic agents, and astringents, and also optionally calmatives and anti-glycation agents, and mixtures thereof.

The cleansing compositions according to the invention will also advantageously comprise at least one foaming surfactant.

At least one active agent chosen from anti-acne agents, cicatrizing agents and anti-inflammatory agents will preferably be chosen for caring for and/or making up acne-prone skin.

For slimming care of the body, he will preferably choose an active agent chosen from slimming active agents and active agents for promoting the cutaneous microcirculation.

The composition may also comprise at least one additional ingredient for complementing the biological effect of these active agents or for providing an immediate visual effect; mention may be made especially of matting agents, fillers with a soft-focus effect, fluorescers, agents for promoting the naturally pinkish coloration of the skin, and abrasive fillers or exfoliants.

According to a first mode, the composition according to the invention comprises at least one moisturizer.

According to another mode, the composition according to the invention comprises at least one desquamating agent, in particular at least 5-n-octanoylsalicylic acid, optionally in combination with salicylic acid.

According to another mode, the composition according to the invention comprises at least one agent for improving the barrier function.

In particular, the combination between the phenanthrenol compound and the agent for improving the barrier function and/or the composition containing it is used according to the invention for improving the barrier function and/or the skin cicatrization process.

According to another mode, the composition according to the invention comprises at least one depigmenting agent.

In particular, the combination between the phenanthrenol compound and the depigmenting agent and/or the composition containing it is used according to the invention for promoting the bleaching and/or depigmentation of the skin.

According to another mode, the composition according to the invention comprises at least one antioxidant.

In particular, the combination between the phenanthrenol compound and the antioxidant and/or the composition containing it is used according to the invention for improving the antioxidant effect of the composition.

According to another mode, the composition according to the invention comprises at least one dermo-decontracting agent.

In particular, the combination between the phenanthrenol compound and the dermo-decontracting agent and/or the composition containing it is used according to the invention for promoting dermo-decontraction and/or dermo-relaxation of the skin, and thus for preventing and/or treating expression wrinkles on the face and in particular on the forehead and between the eyebrows.

According to another mode, the composition according to the invention comprises at least one anti-glycation agent.

According to another mode, the composition according to the invention comprises at least one agent for stimulating the synthesis of dermal and/or epidermal macromolecules and/or for preventing their degradation.

According to another mode, the composition according to the invention comprises at least one agent for stimulating fibroblast or keratinocyte proliferation and/or keratinocyte differentiation.

According to another mode, the composition according to the invention comprises at least one agent for promoting the maturation of the horny envelope.

According to another mode, the composition according to the invention comprises at least one NO-synthase inhibitor.

According to another mode, the composition according to the invention comprises at least one peripheral benzodiazepine receptor (PBR) antagonist.

According to another mode, the composition according to the invention comprises at least one agent for increasing the activity of the sebaceous glands.

According to another mode, the composition according to the invention comprises at least one agent for stimulating the energy metabolism of cells.

According to another mode, the composition according to the invention comprises at least one tensioning agent.

According to another mode, the composition according to the invention comprises at least one lipo-restructuring agent.

In particular, the combination between the phenanthrenol compound and a lipo-restructuring agent and/or the composition containing it is used according to the invention for improving the lipostructure of the skin, in particular for reducing the age-related hollowing of the face.

According to another mode, the composition according to the invention comprises at least one slimming agent.

According to another mode, the composition according to the invention comprises at least one agent for promoting the cutaneous microcirculation.

In particular, the combination between the phenanthrenol compound and the agent for promoting the cutaneous microcirculation and/or the composition containing it is used according to the invention to improve the appearance of the area around the eyes, in particular to reduce the shadows under the eyes.

According to another mode, the composition according to the invention comprises at least one calmative and/or anti-irritant, in particular chosen from glycyrrhetinic acid and salts and derivatives thereof, extract of Eperua falcata bark, and a mixture thereof.

In particular, the combination between the phenanthrenol compound and the calmative and/or anti-irritant and/or the composition containing it is used according to the invention for soothing the skin.

According to another mode, the composition according to the invention comprises at least one sebo-regulating or anti-seborrhoeic agent, in particular chosen from zinc salts, copper derivatives, phthalimidoperoxyhexanoic or phthalimidoperoxycaproic acid (PAP), and mixtures thereof.

According to another mode, the composition according to the invention comprises at least one astringent, in particular a cinnamon extract.

According to another mode, the composition according to the invention comprises at least one cicatrizing agent.

According to another mode, the composition according to the invention comprises at least one anti-inflammatory agent.

According to another mode, the composition according to the invention comprises at least one anti-acne agent.

According to another mode, the composition according to the invention comprises at least one matting agent.

According to another mode, the composition according to the invention comprises at least one filler with a soft-focus effect.

According to another mode, the composition according to the invention comprises at least one fluorescer.

According to another mode, the composition according to the invention comprises at least one agent for promoting the naturally pinkish coloration of the skin.

According to another mode, the composition according to the invention comprises at least abrasive fillers or exfoliants.

Examples of such compounds are described below.

### 1. Moisturizers or Humectants

Moisturizers or humectants that may especially be mentioned include glycerol and derivatives thereof, urea and derivatives thereof, especially Hydrovanc® sold by National Starch, lactic acid, hyaluronic acid, AHAs, BHAs, sodium pidolate, xylitol, serine, sodium lactate, ectoin and derivatives thereof, chitosan and derivatives thereof, collagen, plankton, an extract of Imperata cylindra sold under the name Moist 24® by the company Sederma, acrylic acid homopolymers, for instance Lipidure-HM® from NOF Corporation, beta-glucan from Mibelle-AG-Biochemistry; a mixture of passion-flower oil, apricot oil, corn oil and rice bran oil sold by Nestle under the name NutraLipids®; a C-glycoside derivative such as those described in patent application WO 02/051 828 and in particular C-β-D-xylopyranoside-2-hydroxypropane in the form of a solution containing 30% by weight of active material in a water/propylene glycol mixture (60/40% by weight) such as the product sold by Chimex under the trade name Mexoryl SBB®; an oil of musk rose sold by Nestle; an oil of the microalga Prophyridium cruentum enriched with zinc, sold by Vincience under the name Algualane Zinc®; spheres of collagen and of chondroitin sulfate of marine of origin (Atelocollagen) sold by the company Engelhard Lyon under the name Marine Filling Spheres; hyaluronic acid spheres such as those sold by the company Engelhard Lyon; and arginine and derivatives such as arginine pidolate.

The moisturizer that will preferably be used is chosen from urea and derivatives thereof, especially Hydrovance® sold by National Starch, hyaluronic acid, AHAs, BHAs, acrylic acid homopolymers, for instance Lipidure-HM® from NOF Corporation, beta-glucan from Mibelle-AG-Biochemistry; a mixture of passionflower oil, apricot oil, corn oil and rice bran oil sold by Nestle under the name NutraLipids®; a C-glycoside derivative such as those described in patent application WO 02/051 828 and in particular C-β-D-xylopyranoside-2-hydroxypropane in the form of a solution containing 30% by weight of active material in a water/propylene glycol mixture (60/40% by weight) such as the product sold by Chimex under the trade name Mexoryl SBB®; an oil of musk rose sold by Nestle; an oil of the microalga Prophyridium cruentum enriched with zinc, sold by Vincience under the name Algualane Zinc®; spheres of collagen and of chondroitin sulfate of marine of origin (Atelocollagen) sold by the company Engelhard Lyon under the name Marine Filling Spheres; hyaluronic acid spheres such as those sold by the company Engelhard Lyon; and arginine and derivatives such as arginine PCA.

According to one particular mode, a C-glycoside derivative such as those described in patent application WO 02/051 828 is used, and in particular C-β-D-xylopyranoside-2-hydroxypropane in the form of a solution containing 30% by weight of active material in a water/propylene glycol mixture (60%/40% by weight), such as the product sold by Chimex under the trade name Mexoryl SBB®.

### 2. Desquamating Agents

The term “desquamating agent” means any compound capable of acting:
either directly on desquamation by promoting exfoliation, such as β-hydroxy acids (BHA), in particular salicylic acid and derivatives thereof (including 5-n-octanoylsalicylic acid, also known as capryloyl salicylic acid as the INCI name); α-hydroxy acids (AHA), such as glycolic acid, citric acid, lactic acid, tartaric acid, malic acid or mandelic acid; 8-hexadecene-1,16-dicarboxylic acid or 9-octadecenedioic acid; gentisic acid and derivatives thereof; oligofucoses; cinnamic acid; Saphora japonica extract; resveratrol, and certain jasmonic acid derivatives;
or on the enzymes involved in the desquamation or degradation of corneodesmosomes, glycosidases, stratum corneum chymotryptic enzyme (SCCE) or other proteases (trypsin, chymotrypsin-like). Mention may be made of aminosulfonic compounds and in particular 4-(2-hydroxyethyl)piperazine-1-propanesulfonic acid (HEPES); 2-oxothiazolidine-4-carboxylic acid (procysteine) derivatives; derivatives of α-amino acids of glycine type (as described in EP-0 852 949, and also sodium methyl glycine diacetate sold by BASF under the trade name Trilon M); honey; O-octanoyl-6-D-maltose and N-acetylglucosamine.

As other desquamating agents that may be used in the composition according to the invention, mention may be made of:
oligofructoses, laminaria extracts, O-linoleyl-6D-glucose, (3-hydroxy-2-pentylcyclopentyl)acetic acid, glycerol trilactate, O-octanyl-6′-D-maltose, S-carboxymethylcysteine, siliceous derivatives of salicylate such as those described in patent EP 0 796 861, oligofucases such as those described in patent EP 0 218 200, 5-acyl salicylic acid salts, active agents with effects on transglutaminase, as in patent EP 0 899 330,
extract of the flowers of ficus Opuntia indica (Exfolactive® from Silab),
8-hexadecene-1,16-dicarboxylic acid,
esters of glucose and of vitamin F, and
mixtures thereof.

Preferred desquamating agents that may be mentioned include β-hydroxy acids such as 5-n-octanoyl salicylic acid; urea; glycolic acid, citric acid, lactic acid, tartaric acid, malic acid or mandelic acid; 4-(2-hydroxyethyl)piperazine-1-propanesulfonic acid (HEPES); extract of Saphora japonica; honey; N-acetyl glucosamine; sodium methyl glycine diacetate, and mixtures thereof.

Even more preferentially, a desquamating agent chosen from 5-n-octanoyl salicylic acid; lactic acid; urea; 4-(2-hydroxyethyl)piperazine-1-propanesulfonic acid (HEPES); extract of Saphora japonica; honey; N-acetyl glucosamine; sodium methyl glycine diacetate, and mixtures thereof, will be used in the compositions of the invention.

5-n-octanoylsalicylic acid, 4-(2-hydroxyethyl)piperazine-1-propanesulfonic acid (HEPES) and mixtures thereof will be used as the most preferred desquamating agents.

According to one particular mode of the invention, the composition comprises at least one phenanthrenol compound, in particular Totarol®, and at least 5-n-octanoylsalicylic acid. According to one preferred mode, the composition contains at least one phenanthrenol compound, in particular Totarol®, at least 5-n-octanoylsalicylic acid and salicylic acid.

The phenanthrenol compound and the 5-n-octanoylsalicylic acid may advantageously be encapsulated in porous Nylon-12 particles as described previously.

### 3. Agents for Improving the Barrier Function

As agents for improving the barrier function, mention may be made especially of arginine, an extract of Thermus thermophilus such as Venuceane® from Sederma, an extract of the rhizome of wild yam (Dioscorea villosa) such as Actigen Y® from Active Organics, plankton extracts, for instance Omega Plankton® from Secma, yeast extracts, for instance Relipidium® from Coletica, a chestnut extract such as Recoverine® from Silab, a cedar bud extract such as Gatuline Zen® from Gattefosse, sphingosines, for instance salicyloyl sphingosine sold under the name Phytosphingosine® SLC by the company Degussa, a mixture of xylitol, polyxylityl glycoside and xylitan, for instance Aquaxyl® from SEPPIC, extracts of Solanacea plants, for instance Lipidessence® from Coletica; omega-3 unsaturated oils such as musk rose oils; and mixtures thereof.

Mention may also be made especially of ceramides or derivatives thereof, in particular ceramides of type 2 (for instance N-oleoyldihydrosphingosine), of type 3 (for instance stearoyl-4-hydroxysphinganine, as the INCI name) and of type 5 (for instance N-2-hydroxypalmitoyldihydrosphingosine, having the INCI name: hydroxypalmitoyl sphinganine), sphingoid-based compounds, glycosphingolipids, phospholipids, cholesterol and derivatives thereof, phytosterols, essential fatty acids, diacylglycerol, 4-chromanone and chromone derivatives, petroleum jelly, lanolin, shea butter, cocoa butter, lanolin and PCA salts.

As preferred agents having a restructuring effect on the barrier function, mention will be made of an extract of Thermus thermophilus, an extract of wild yam rhizome (Dioscorea villosa), a yeast extract, a chestnut extract, a cedar bud extract, arginine, ceramides especially of type 3 and 5; and mixtures thereof.

Serine or arginine, or a mixture thereof, will preferably be used.

### 4. Depigmenting Agents

Depigmenting agents that may especially be mentioned include vitamin C and derivatives thereof and especially vitamin CG, CP and 3-o ethyl vitamin C, alpha and beta arbutin, ferulic acid, lucinol and derivatives thereof, kojic acid, resorcinol and derivatives thereof, tranexamic acid and derivatives thereof, gentisic acid, homogentisate, methyl gentisate or homogentisate, dioic acid, calcium D-pantheteine sulfonate, lipoic acid, ellagic acid, vitamin B3, linoleic acid and derivatives thereof, ceramides and homologues thereof, plant derivatives, for instance camomile, bearberry, the aloe family (vera, ferox, bardensis), mulberry or skullcap; a kiwi fruit (Actinidia chinensis) juice sold by Gattefosse, an extract of Paeonia suffruticosa root, such as the product sold by the company Ichimaru Pharcos under the name Botanpi Liquid B®, an extract of brown sugar (Saccharum officinarum), such as the extract of molasses sold by the company Taiyo Kagaku under the name Molasses Liquid, jasmonic acid and derivatives thereof without this list being exhaustive.

Vitamin C and derivatives thereof and especially vitamin CG, CP and 3-o ethyl vitamin C, alpha and beta arbutin, ferulic acid, kojic acid, resorcinol and derivatives thereof, calcium D-pantheteine sulfonate, lipoic acid, ellagic acid, vitamin B3, a kiwi fruit (Actinidia chinensis) juice sold by Gattefosse, and an extract of Paeonia suffruticosa root, such as the product sold by the company Ichimaru Pharcos under the name Botanpi Liquid B®, will be used as preferred depigmenting agents.

According to one preferred mode, the depigmenting agent is chosen from vitamin CG, ferulic acid, kojic acid, resorcinol and derivatives thereof, calcium D-pantheteine sulfonate, lipoic acid, ellagic acid and jasmonic acid and derivatives thereof.

More preferably, ellagic acid or vitamin CG will be used.

### 5. Antioxidants

Mention may be made especially of tocopherol and esters thereof, in particular tocopheryl acetate; ascorbic acid and derivatives thereof, in particular magnesium ascorbyl phosphate and ascorbyl glucoside; ferulic acid; serine; ellagic acid, polyphenols, tannins, tannic acid, epigallocatechins and natural extracts containing them, anthocyans, rosemary extracts, olive leaf extracts, for instance those from the company Silab, green tea extracts, resveratrol and derivatives thereof, ergothioneine, N-acetylcysteine, an extract of the brown alga Pelvetia caniculata, for instance Pelvetiane® from Secma, chlorogenic acid, biotin, chelating agents, such as BHT and BHA, N,N′-bis(3,4,5-trimethoxybenzyl)ethylenediamine and salts thereof; idebenone, plant extracts, for instance Pronalen Bioprotect™ from the company Provital; coenzyme Q10, bioflavonoids, SODs, phytanetriol, lignans, melatonin, pidolates, glutathione, caprylyl glycol, phloretin, a jasmine extract such as the product sold by Silab under the name Helisun®; hesperitin laurate such as Flavagrum PEG® from the company Engelhard Lyon; an extract of Paeonia suffruticosa root, such as the product sold by the company Ichimaru Pharcos under the name Botanpi Liquid B®; an extract of lychee such as the extract of lychee pericarp sold by the company Cognis under the name Litchiderm LS 9704®, an extract of pomegranate fruit (Punica granatum), such as the product sold by the company Draco Natural Products.

Other anti-ageing agents that may be mentioned include DHEA and derivatives thereof, boswellic acid, rosemary extracts, carotenoids (β-carotene, zeaxanthin and lutein), cysteic acid, copper derivatives and jasmonic acid.

Preferred antioxidants that will especially be used include ferulic acid; serine; biotin, chelating agents such as BHT, BHA, N,N′-bis(3,4,5-trimethoxybenzyl)ethylenediamine and salts thereof; caprylyl glycol, phloretin, a jasmine extract such as the product sold by Silab under the name Helisun®; hesperitin laurate such as Flavagrum PEG® from the company Engelhard Lyon; an extract of Paeonia suffruticosa root, such as the product sold by the company Ichimaru Pharcos under the name Botanpi Liquid B®.

### 6. Dermo-Relaxing or Dermo-Decontracting Agents

Examples that may be mentioned include manganese gluconate and other salts, adenosine, alverine citrate and salts thereof, lysine, an extract of Iris pallida, a hexapeptide (Argeriline R from Lipotec) or sapogenins, for instance wild yam and the carbonyl amines described in patent application EP 1 484 052. Examples of sapogenins that may be mentioned include those described in patent application WO 02/47650, in particular wild yam, the diosgenin extracted especially from Dioscorea opposita or any extract naturally containing or containing after treatment one or more sapogenins (wild yam rhizome, agave leaf, which contains hecogenin and tigogenin, extracts of Liliacea plants and more particularly yucca or smilax containing smilagenin and sarsapogenin, or sarsaparilla) or Actigen Y from the company Actives Organics; or ginger.

Mention may also be made of DMAE (dimethyl MEA), extracts of sea fennel, of rockrose, of helichrysum, of aniseed, of paracress, and an extract of Acmella oleracea, for instance Gatuline® from Gattefosse.

Preferred dermo-relaxing agents that will be mentioned include adenosine, manganese gluconate, wild yam, sea fennel, glycine and alverine.

### 7. Anti-Glycation Agents

The term “anti-glycation agent” means a compound that prevents and/or reduces the glycation of skin proteins, in particular dermal proteins such as collagen.

Anti-glycation agents that may especially be mentioned include extracts of plants of the Ericacea family, such as an extract of blueberry (Vaccinium angustifolium or Vaccinium myrtillus), for example the product sold under the name Blueberry Herbasol Extract PG by the company Cosmetochem, ergothioneine and derivatives thereof, hydroxystilbenes and derivatives thereof, such as resveratrol and 3,3′,5,5′-tetrahydroxystilbene (these anti-glycation agents are described in patent applications FR 2 802 425, FR 2 810 548, FR 2 796 278 and FR 2 802 420, respectively), dihydroxystilbenes and derivatives thereof, polypeptides of arginine and of lysine such as the product sold under the name Amadorine® by the company Solabia, carsinine hydrochloride (sold by Exsymol under the name Alistin®), an extract of Helianthus annuus, for instance Antiglyskin® from Silab, wine extracts such as the extract of powdered white wine on a maltodextrin support sold under the name Vin blanc deshydraté 2F by the company Givaudan, thioctic acid (or alpha-lipoic acid), a mixture of extract of bearberry and of marine glycogen, for instance Aglycal LS 8777® from Laboratoires Sérobiologiques, and an extract of black tea, for instance Kombuchka® from Sederma, and mixtures thereof.

Preferred anti-glycation agents that will be mentioned include extracts of blueberry (Vaccinium myrtillus) and extracts of black tea.

8. Agents for Stimulating the Synthesis of Dermal and/or Epidermal Macromolecules and/or for Preventing their Degradation

Among the active agents for stimulating the dermal macromolecules or for preventing their degradation, mention may be made of those acting:
either on collagen synthesis, such as extracts of Centella asiatica, asiaticosides and derivatives thereof; ascorbic acid or vitamin C and derivatives thereof; synthetic peptides such as iamin, biopeptide CL or palmitoyl oligopeptide sold by the company Sederma; peptides extracted from plants, such as the soybean hydrolysate sold by the company Coletica under the trade name Phytokine®; rice peptides such as Nutripeptide® from Silab, methylsilanol mannuronate such as Algisium C® sold by Exsymol; plant hormones such as auxins and lignans; folic acid; and an extract of Medicago sativa (alfalfa) such as the product sold by Silab under the name Vitanol®; a peptide extract of hazelnut such as the product sold by the company Solabia under the name Nuteline C®; and arginine;
or on the inhibition of collagen degradation, in particular agents acting on the inhibition of metalloproteases (MMP) more particularly such as MMP 1, 2, 3 and 9. Mention may be made of: retinoids and derivatives, extracts of Medicago sativa such as Vitanol® from Silab, an extract of Aphanizomenon flosaquae (Cyanophyceae) sold under the name Lanablue® by Atrium Biotechnologies, oligopeptides and lipopeptides, lipoamino acids, the malt extract sold by the company Coletica under the trade name Collalift®; blueberry or rosemary extracts; lycopene; isoflavones, derivatives thereof or plant extracts containing them, in particular extracts of soybean (sold, for example, by the company Ichimaru Pharcos under the trade name Flavosterone SB®), of red clover, of flax or of kakkon; an extract of lychee; Dipalmitoyl Hydroxyproline sold by SEPPIC under the name Sepilift DPHP®: Baccharis genistelloides or Baccharine sold by Silab, an extract of moring a such as Arganyl LS 9781® from Cognis; the sage extract described in patent application FR-A-2 812 544 from the Labiatae family (Salvia officinalis from the company Flacksmann), an extract of rhododendron, a blueberry extract, and an extract of Vaccinium myrtillus such as those described in patent application FR-A-2 814 950;
or on the synthesis of molecules belonging to the elastin family (elastin and fibrillin), such as: retinol and derivatives, in particular retinyl palmitate; the extract of Saccharomyces cerevisiae sold by the company LSN under the trade name Cytovitin®; and the extract of the alga Macrocystis pyrifera sold by the company Secma under the trade name Kelpadelie®; a peptide extract of hazelnut such as the product sold by the company Solabia under the trade name Nuteline C®;
or on inhibition of elastin degradation, such as the peptide extract of seeds of Pisum sativum sold by the company LSN under the trade name Parelastyl®; heparinoids; and the N-acylamino acid compounds described in patent application WO 01/94381, such as {2-[acetyl(3-trifluoromethylphenyl)amino]-3-methylbutyrylamino}acetic acid, also known as N—[N-acetyl, N′-(3-trifluoromethyl)phenylvalyl]glycine, or N-acetyl-N-[3-(trifluoromethyl)phenyl]valyl glycine or acetyl trifluoromethyl phenyl valylglycine, or an ester thereof with a C₁-C₆ alcohol; an extract of rice peptides such as Colhibin® from Pentapharm, or an extract of Phyllanthus emblica such as Emblica® from Rona;
or on the synthesis of glycosaminoglycans, such as the product of fermentation of milk with Lactobacillus vulgaris, sold by the company Brooks under the trade name Biomin Yoghurt®; the extract of the brown alga Padina pavonica sold by the company Alban Müller under the trade name HSP3®; the Saccharomyces cerevisiae extract available especially from the company Silab under the trade name Firmalift® or from the company LSN under the trade name Cytovitin®; an extract of Laminaria ochroleuca such as Laminaine® from Secma; essence of Mamaku from Lucas Meyer, and an extract of Cress (Odraline® from Silab); a C-glycoside derivative such as those described in patent application WO 02/051 828 and in particular C-β-D-xylopyranoside-2-hydroxypropane in the form of a solution containing 30% by weight of active material in a water/propylene glycol mixture (60%/40% by weight), such as the product manufactured by Chimex under the trade name Mexoryl SBB®;
or on the synthesis of fibronectin, such as the extract of the zooplankton Salina sold by the company Seporga under the trade name GP4G®; the yeast extract available especially from the company Alban Müller under the trade name Drieline®; and the palmitoyl pentapeptide sold by the company Sederma under the trade name Matrixyl®.

Among the active agents for stimulating epidermal macromolecules, such as fillagrin and keratins, mention may be made especially of the extract of lupin sold by the company Silab under the trade name Structurine®; the extract of Fagus sylvatica beech buds sold by the company Gattefosse under the trade name Gatuline® RC; and the extract of the zooplankton Salina sold by the company Seporga under the trade name GP4G®; the copper tripeptide from Procyte; a peptide extract of Voandzeia substerranea such as the product sold by the company Laboratoires Sérobiologiques under the trade name Filladyn LS 9397®.

Preferably, an active agent that stimulates the synthesis of dermal and/or epidermal macromolecules and/or that prevents their degradation, chosen from agents for stimulating the synthesis of glycosaminoglycans, agents for inhibiting elastin degradation, agents for stimulating fibronectin synthesis, agents for stimulating the synthesis of epidermal macromolecules, and mixtures thereof, will preferably be used.

Even more preferentially, an active agent that stimulates the synthesis of the glycosaminoglycans, chosen from an extract of the brown alga Padina pavonica, an extract of Saccharomyces cerevisiae, an extract of Laminaria ochroleuca, essence of Mamaku, an extract of cress, a C-glycoside derivative such as those described in patent application WO 02/051 828 and in particular C-β-D-xylopyranoside-2-hydroxypropane in the form of a solution containing 30% by weight of active material in a water/propylene glycol mixture (60%/40% by weight), such as the product manufactured by Chimex under the trade name Mexoryl SBB® and mixtures thereof, will even more preferentially be used.

According to one particular mode, a C-glycoside derivative or essence of Mamaku will be used.

According to one preferred mode, essence of Mamaku will be used.

As preferred active agents for stimulating the synthesis of dermal and/or epidermal macromolecules and/or for preventing their degradation, mention may be made of:

synthetic peptides such as iamin, the biopeptide CL or palmitoyloligopeptide sold by the company Sederma; peptides extracted from plants, such as the soybean hydrolysate sold by the company Coletica under the trade name Phytokine®; rice peptides such as Nutripeptide® from Silab, methylsilanol mannuronate such as Algisium C® sold by Exsymol; folic acid; an extract of Medicago sativa (alfalfa), such as the product sold by Silab under the name Vitanol®; a peptide extract of hazelnut, such as the product sold by the company Solabia under the name Nuteline C®; arginine; an extract of Aphanizomenon flos-aquae (Cyanophyceae) sold under the name Lanablue® by Atrium Biotechnologies, the malt extract sold by the company Coletica under the trade name Collalift®, lycopene; an extract of lychee; an extract of moring a such as Arganyl LS 9781® from Cognis; an extract of Vaccinium myrtillus such as those described in patent application FR-A-2 814 950; retinol and derivatives thereof, in particular retinyl palmitate; the extract of Saccharomyces cerevisiae sold by the company LSN under the trade name Cytovitin®; a peptide extract of hazelnut such as the product sold by the company Solabia under the name Nuteline C®; {2-[acetyl(3-trifluoromethylphenyl)amino]-3-methylbutyrylamino}-acetic acid, also known as N—[N-acetyl, N′-(3-trifluoromethyl)phenylvalyl]glycine, or N-acetyl-N-[3-(trifluoromethyl)phenyl]valyl glycine or acetyl trifluoromethyl phenyl valylglycine, or an ester thereof with a C₁-C₆ alcohol; an extract of rice peptides such as Colhibin® from Pentapharm, or an extract of Phyllanthus emblica such as Emblica® from Rona; the extract of the brown alga Padina pavonica sold by the company Alban Müller under the trade name HSP3®; the extract of Saccharomyces cerevisiae available especially from the company Silab under the trade name Firmalift® or from the company LSN under the trade name Cytovitin®; an extract of Laminaria ochroleuca such as Laminaine® from Secma; a C-glycoside derivative such as those described in patent application WO 02/051 828 and in particular C-β-D-xylopyranoside-2-hydroxypropane in the form of a solution containing 30% by weight of active material in a water/propylene glycol mixture (60%/40% by weight), such as the product manufactured by Chimex under the trade name Mexoryl SBB®; the essence of Mamaku from Lucas Meyer, the extract of lupin sold by the company Silab under the trade name Structurine®; the extract of Fagus sylvatica beech buds sold by the company Gattefosse under the trade name Gatuline® RC.
9. Agents for Stimulating Fibroblast or Keratinocyte Proliferation and/or Keratinocyte Differentiation

The agents for stimulating fibroblast proliferation that may be used in the composition according to the invention may be chosen, for example, from plant proteins or polypeptides, extracted especially from soybean (for example a soybean extract sold by the company LSN under the name Eleseryl SH-VEG 8® or sold by the company Silab under the trade name Raffermine®); an extract of hydrolysed soybean proteins such as Ridulisse® from Silab; and plant hormones such as gibberellins and cytokinins; a peptide extract of hazelnut such as the product sold by the company Solabia under the name Nuteline C®.

Preferably, an agent that promotes keratinocyte proliferation and/or differentiation will be used.

The agents for stimulating keratinocyte proliferation that may be used in the composition according to the invention especially comprise adenosine; phloroglucinol, the extract of Hydrangea macrophylla leaves, for instance Amacha Liquid E® from Ichimaru Pharcos, a yeast extract such as Stimoderm® from CLR; the extract of Larrea divaricata such as Capislow® from Sederma, mixtures of extract of papaya, of olive leaves and of lemon, such as Xyleine® from Vincience, the extract of Hydrangea macrophylla leaves, for instance Amacha Liquid E® from Ichimaru Pharcos, retinol and esters thereof, including retinyl palmitate, phloroglucinol, the nut cake extracts sold by the Gattefosse and the extracts of Solanum tuberosum such as Dermolectine® sold by Sederma.

Among the agents for stimulating keratinocyte differentiation are, for example, minerals such as calcium; a peptide extract of lupin, such as the product sold by the company Silab under the trade name Structurine®; sodium beta-sitosteryl sulfate, such as the product sold by the company Seporga under the trade name Phytocohesine®; and a water-soluble extract of corn, such as the product sold by the company Solabia under the trade name Phytovityl®; a peptide extract of Voandzeia substerranea such as the product sold by the company Laboratoires Sérobiologiques under the trade name Filladyn LS 9397®; and lignans such as secoisolariciresinol, and retinol and esters thereof, including retinyl palmitate.

As agents for stimulating keratinocyte proliferation and/or differentiation, mention may also be made of oestrogens such as oestradiol and homologues; cytokines.

As preferred active agents for stimulating fibroblast or keratinocyte proliferation and/or keratinocyte differentiation, mention will be made of plant proteins or polypeptides, extracted especially from soybean (for example a soybean extract sold by the company LSN under the name Eleseryl SH-VEG 8® or sold by the company Silab under the trade name Raffermine®); an extract of hydrolysed soybean proteins such as Ridulisse® from Silab; a peptide extract of hazelnut such as the product sold by the company Solabia under the name Nuteline C®; adenosine; phloroglucinol, a yeast extract such as Stimoderm® from CLR; a peptide extract of lupin such as the product sold by the company Silab under the trade name Structurine®; a water-soluble corn extract, such as the product sold by the company Solabia under the trade name Phytovityl®; a peptide extract of Voandzeia substerranea, such as the product sold by the company Laboratoires Sérobiologiques under the trade name Filladyn LS 9397®; retinol and esters thereof, including retinyl palmitate.

### 10. Agents for Promoting the Maturation of the Horny Envelope

Agents that participate in the maturation of the horny envelope, which becomes impaired with age and induces a decrease in transglutaminase activity, may be used in the compositions of the invention. Examples that may be mentioned include urea and derivatives thereof and in particular Hydrovance® from National Starch and the other active agents mentioned in L'Oréal patent application FR 2 877 220.

### 11. NO-Synthase Inhibitors

The agent with an inhibitory action on NO synthase may be chosen from OPCs (procyannidol oligomers); plant extracts of the species Vitis vinifera sold especially by the company Euromed under the name “Leucocyanidines de raisins extra”, or by the company Indena under the name Leucoselect®, or finally by the company Hansen under the name “Extrait de marc de raisin”; plant extracts of the species Olea europaea preferably obtained from olive tree leaves and sold especially by the company Vinyals in the form of a dry extract, or by the company Biologia & Technologia under the trade name Eurol BT; and plant extracts of the species Gingko biloba, preferably a dry aqueous extract of this plant sold by the company Beaufour under the trade name “Ginkgo biloba extrait standard”, and mixtures thereof.

### 12. Peripheral Benzodiazepine Receptor (PBR) Antagonists

Mention may be made, for example, of 1-(2-chlorophenyl)-N-(1-methylpropyl)-3-isoquinoline carboxamide; the compounds described in patent applications WO 03/030 937 and WO 03/068 753, pyridazino[4,5-b]indole-1-acetamide derivatives of general formula (VII) as described in document WO 00/44384.

### 13. Agents for Increasing the Activity of the Sebaceous Glands

Mention may be made, for example, of methyl dehydrojasmonate, hecogenin, hedione and O-linoleyl-6D-glucose, and mixtures thereof.

### 14. Agents for Stimulating the Energy Metabolism of Cells

The active agent for stimulating the energy metabolism of cells may be chosen, for example, from biotin, an extract of Saccharomyces cerevisiae such as Phosphovital® from Sederma, the mixture of sodium, manganese, zinc and magnesium salts of pyrrolidonecarboxylic acid, for instance Physiogenyl® from Solabia, a mixture of zinc, copper and magnesium gluconate, such as Sepitonic M3® from SEPPIC, and mixtures thereof; a beta-glucan derived from Saccharomyces cerevisiae, such as the product sold by the company Mibelle AG Biochemistry.

### 15. Tensioning Agents

The term “tensioning agent” that may be used according to the invention means compounds liable to have a tensioning effect, i.e. being able to make the skin taut.

According to the invention, the term “tensioning agent” generally means any polymer that is soluble or dispersible in water at a temperature ranging for example from 25° C. to 50° C. at a concentration of 7% by weight in water or at the maximum concentration at which a medium of uniform appearance is formed and producing at this concentration of 7% or at this maximum concentration in water a shrinkage of more than 15% in the test described below.

The maximum concentration at which a medium of uniform appearance forms is determined to within ±10% and preferably to within ±5%.

The expression “medium of uniform appearance” means a medium that does not contain any aggregates that are visible to the naked eye.

For the determination of the maximum concentration, the tensioning agent is gradually added to the water with deflocculating stirring at a temperature ranging for example from 25° C. to 50° C., and the mixture is then stirred for one hour. The mixture thus prepared is then examined after 24 hours to see if it is of uniform appearance (absence of aggregates visible to the naked eye).

The tensioning effect may be characterized by an in vitro shrinkage test.

A homogeneous mixture of the tensioning agent in water, at a concentration of 7% by weight or at the maximum concentration defined above, is prepared beforehand and as described previously.

30 μl of the homogeneous mixture are placed on a rectangular sample (10×40 mm, thus having an initial width L₀ of 10 mm) of elastomer with a modulus of 20 MPa and a thickness of 100 μm.

After drying for 3 hours at 22±3° C. and 40±10% relative humidity RH, the elastomer sample has a shrunken width, noted L₃ₕ, due to the tension exerted by the applied tensioning agent.

The tensioning effect (TE) of the polymer is then quantified in the following manner:

‘TE’ = (L₀ − L₃ₕ/L₀) × 100 as %
with L₀ = initial width 10 mm
and L₃ₕ = width after 3 hours
of drying

The tensioning agent may be chosen from:

a) plant or animal proteins and hydrolysates thereof;
b) polysaccharides of natural origin;
c) mixed silicates;
d) colloidal particles of mineral fillers;
e) synthetic polymers;
and mixtures thereof.

A person skilled in the art will know how to choose, from the chemical categories listed above, the materials corresponding to the tensioning test as described below.

Mention may be made especially of:

(a) plant proteins and protein hydrolysates, in particular of corn, rye, wheat, buckwheat, sesame, spelt, pea, bean, lentil, soybean and lupin,
(b) polysaccharides of natural origin, especially (a) polyholosides, for example (i) in the form of starch derived especially from rice, corn, potato, cassaya, pea, wheat, oat, etc. or (ii) in the form of carrageenans, alginates, agars, gellans, cellulose polymers and pectins, advantageously as an aqueous dispersion of gel microparticles, and (b) latices consisting of shellac resin, sandarac gum, dammar resins, elemi gums, copal resins, cellulose derivatives, and mixtures thereof,
(c) mixed silicates, especially phyllosilicates and in particular Laponites,
(d) colloidal particles of mineral fillers with a number-average diameter of between 0.1 and 100 nm and preferably between 3 and 30 nm, and chosen, for example, from: silica, silica-alumina composites, cerium oxide, zirconium oxide, alumina, calcium carbonate, barium sulfate, calcium sulfate, zinc oxide and titanium dioxide. As silica-alumina composite colloidal particles that may be used in the compositions according to the invention, examples that may be mentioned include those sold by the company Grace under the names Ludox AM, Ludox AM-X 6021, Ludox HSA and Ludox TMA,
(e) synthetic polymers, such as polyurethane latices or acrylic-silicone latices, in particular those described in patent application EP-1 038 519, such as a polydimethylsiloxane grafted with propylthio(polymethyl acrylate), propylthio(polymethyl methacrylate) and propylthio(polymethacrylic acid), or alternatively a polydimethylsiloxane grafted with propylthio(polyisobutyl methacrylate) and propylthio(polymethacrylic acid). Such grafted silicone polymers are especially sold by the company 3M under the trade names VS 80, VS 70 and L021.

The tensioning agent will be present in the composition in an amount that is effective for obtaining the desired biological effect according to the invention.

By way of example, the tensioning agent may be included in the composition according to the invention in a content ranging for example from 0.01% to 30% by weight of active material and preferably from 1% to 30% by weight of active material relative to the total weight of the composition.

The term “active material” is intended to exclude the medium in which the tensioning agent may be dissolved or dispersed in its commercial form, for example in the case of dispersions of colloidal particles.

It is also possible, especially for complementing and/or potentiating the effect of tensioning agents, to use agents for increasing the expression of mechanoreceptors, such as agents for increasing the expression of the integrins.

An example that may be mentioned is an extract of rye seed, such as the product sold by Silab under the name Coheliss®.

### 16. Lipo-Restructuring Agents

According to the invention, the term “lipo-restructuring agents” means agents capable of stimulating lipogenesis and of promoting adipocyte differentiation, thus making it possible to prevent or slow down the loss of fat contained in the skin support tissues, which is also referred to as “loss of skin lipostructure”.

The term “skin lipostructure” means the network of fat cells that forms the volumes on which facial skin sits and is moulded.

These agents are intended to:
reduce the loss of cutaneous density and/or the loss of skin lipostructure, in particular on the cheeks and in the area around the eyes, and/or
prevent the collapse and/or hollowing of the volumes of the face, the loss of consistency of the skin and/or its support, in particular on the cheeks and in the area around the eyes, and/or
improve the volumes underlying the skin of the face and/or of the neck, in particular on the cheeks, the oval of the face and the area around the eyes, and/or
improve the density, elasticity and support of the skin, in particular on the cheeks, the oval of the face and the area around the eyes, and/or
remodel the facial features, in particular the oval of the face.

Examples of lipo-restructuring agents that may especially be mentioned include an extract of black tea, such as the extract of fermented black tea sold by Sederma under the name Kombuchka®, and an extract of Artemisia abrotanum, such as the product sold by Silab under the name Pulpactyl

### 17. Slimming Agents

Slimming (lipolytic) agents that may especially be mentioned include theophylline and its derivatives, theobromine, acefylline, aminophylline, chloroethyltheophylline, diprofylline, diniprophylline, etamiphylline and its derivatives, etofylline and proxyphylline; extracts of tea, of coffee, of guarana, of mate, of cola (Cola nitida) and especially the dry extract of guarana fruit (Paulina sorbilis) containing 8% to 10% caffeine; extracts of climbing ivy (Hedera helix), of arnica (Arnica montana L), of rosemary (Rosmarinus officinalis N), of marigold (Calendula officinalis), of sage (Salvia officinalis L), of ginseng (Panax ginseng), of St.-John's wort (Hypericum perforatum), of butcher's-broom (Ruscus aculeatus L), of meadowsweet (Filipendula ulmaria L), of orthosiphon (Orthosiphon stamincus Benth), of birch (Betula alba), of pumpwood and of argan tree, extracts of ginkgo biloba, extracts of horsetail, extracts of escin, extracts of cangzhu, extracts of Chrysanthellum indicum, extracts of diosgenin-rich Dioscorea plants or pure diosgenin or hecogenin and derivatives thereof, extracts of Ballota, extracts of Guioa, of Davallia, of Terminalia, of Barringtonia, of Trema or of Antirobia, the extract of bitter orange pips; an extract of husks of cocoa beans (Theobroma cacao) such as the product sold by Solabia under the name Caobromine®.

### 18. Agents for Promoting the Cutaneous Microcirculation

The active agent acting on the cutaneous microcirculation may be used for preventing dulling of the complexion. It may be chosen, for example, from an extract of maritime pine bark, for instance Pycnogenol® from Biolandes, manganese gluconate (Givobio GMn® from SEPPIC), an extract of Ammi visnaga such as Visnadine from Indena, extract of lupin (Eclaline® from Silab), the protein coupling of hydrolysed wheat/palmitic acid with palmitic acid, such as Epaline 100 from Laboratoires Carilene, the extract of bitter orange blossom (Remoduline® from Silab), vitamin P and derivatives thereof, for instance methyl-4 esculetol sodium monoethanoate sold under the name Permethol® by the company Sephytal, extracts of Ruscus, of common horse chestnut, of ivy, of ginseng and of melilot, caffeine, nicotinate and derivatives thereof, lysine and derivatives thereof, for instance Asparlyne® from Solabia, an extract of black tea such as Kombuchka from Sederma; rutin salts; an extract of the alga Corallina officinalis, such as the product sold by Codif; and mixtures thereof.

As preferred agents for promoting the cutaneous microcirculation, mention will be made of caffeine, an extract of bitter orange blossom, an extract of black tea, rutin salts and an extract of the alga Corallina officinalis.

### 19. Calmatives or Anti-Irritants

The term “calmative” means a compound that can reduce the sensation of stinging, itching or tautness of the skin.

As calmatives that may be used in the composition according to the invention, mention may be made of: procyannidol oligomers, vitamins E, C, B5 and B3, caffeine and derivatives thereof, pentacylic triterpenes and plant extracts containing them, β-glycyrrhetinic acid and salts or derivatives thereof (stearyl glycyrrhetate, 3-stearoyloxyglycyrrhetic acid or glycyrrhetinic acid monoglucuronide) and also plants containing them (e.g.: Glycyrrhiza glabra), oleanolic acid and salts thereof, ursolic acid and salts thereof, boswellic acid and salts thereof, betulinic acid and salts thereof, an extract of Paeonia suffruticosa and/or lactiflora, an extract of Laminaria saccharina, extracts of Centella asiatica, Canola oil, bisabolol, the phosphoric diester of vitamin E and C, for instance Sepivital EPC® from SEPPIC, camomile extracts, allantoin, omega-3 unsaturated oils such as musk rose oil, blackcurrant oil, Ecchium oil, fish oil or beauty-leaf oil, plankton extracts, capryloyl glycine, a mixture of water lily blossom extract and of palmitoylproline, such as the product sold under the name Seppicalm VG® by the company SEPPIC, an extract of Boswellia serrata, an extract of Centipeda cunninghami, such as the product sold under the name Cehami PF® by the company TRI-K Industries, an extract of sunflower seeds, in particular Helioxine® from Silab, an extract of Linum usitatissimum seeds, for instance Sensiline® from Silab, tocotrienols, piperonal, an extract of Epilobium angustifolium, such as the product sold under the name Canadian Willowherb Extract by the company Fytokem Products, Aloe vera, phytosterols, cornflower water, rose water, an extract of mint, in particular of mint leaves, for instance Calmiskin® from Silab, aniseed derivatives, filamentous bacteria, for instance Vitreoscilla filiformis as described in patent EP 761 204, an extract of rose petals, for instance Rose Flower Herbasol® extract from the company Cosmetochem, shea butter, a mixture of the waxy fraction of barley seeds obtained by supercritical CO₂, of shea butter and of argan oil, for instance Stimu-tex AS® from Pentapharm, alkaline-earth metal salts, especially of strontium, a fermented extract of Alteromonas sold under the name Abyssine® by the company Atrium Biotechnologies; spring water from the Vichy basin, such as waters originating from the Célestin, Chomel, Grande-Grille, Hôpital, Lucas and Parc sources, and preferably water from the Lucas source; an extract of Eperua falcata bark, such as the product sold by the company Cognis under the name Eperuline®; an extract of Paeonia suffruticosa root, such as the product sold by the company Ichimaru Pharcos under the name Botanpi Liquid B®; and mixtures thereof.

As preferred calmatives according to the invention, use will be made of:

β-glycyrrhetinic acid and salts or derivatives thereof (stearyl glycyrrhetate, 3-stearoyloxyglycyrrhetic acid or glycyrrhetinic acid monoglucuronide) and also plants containing them (e.g. Glycyrrhiza glabra); ursolic acid and salts thereof; extracts of Centella asiatica, Canola oil, bisabolol; camomile extracts, allantoin; a mixture of extract of water lily blossom and of palmitoylproline, such as the product sold under the name Seppicalm VG® by the company SEPPIC; Aloe vera, rose water, extract of mint, in particular of mint leaves, such as Calmiskin® from Silab, filamentous bacteria such as Vitreoscilla filiformis as described in patent EP 761 204 and sold by Chimex under the name Mexoryl SBG®, an extract of rose petals such as Rose Flower Herbasol® extract from the company Cosmetochem, shea butter, a fermented extract of Alteromonas sold under the name Abyssine® by the company Atrium Biotechnologies; spring water from the Vichy basin, such as waters originating from the Célestin, Chomel, Grande-Grille, Hôpital, Lucas and Parc sources, and preferably water from the Lucas source; an extract of Eperua falcata bark, such as the product sold by the company Cognis under the name Eperuline®; an extract of Paeonia suffruticosa root, such as the product sold by the company Ichimaru Pharcos under the name Botanpi Liquid B®; and mixtures thereof.

According to one preferred mode, β-glycyrrhetinic acid and salts or derivatives thereof, in particular the potassium salt thereof; an extract of Eperua falcata bark such as the product sold by the company Cognis under the name Eperuline®, or a mixture thereof, will be used.

According to one particular mode, the potassium salt of β-glycyrrhetinic acid may be encapsulated in porous Nylon-12 particles containing the phenanthrenol compound in combination with 5-n-octanoylsalicylic acid.

### 20. Sebo-Regulating or Anti-Seborrhoeic Agents

The term “sebo-regulating or anti-seborrhoeic agents” especially means agents capable of regulating the activity of the sebaceous glands.

Mention may be made especially of:
retinoic acid, benzoyl peroxide, sulfur, vitamin B6 (or pyridoxine), selenium chloride and sea fennel;
mixtures of extract of cinnamon, of tea and of octanoylglycine such as Sepicontrol A5 TEA® from SEPPIC;
the mixture of cinnamon, sarcosine and octanoylglycine sold especially by the company SEPPIC under the trade name Sepicontrol A5;
zinc salts such as zinc gluconate, zinc pyrrolidonecarboxylate (or zinc pidolate), zinc lactate, zinc aspartate, zinc carboxylate, zinc salicylate and zinc cysteate;
copper derivatives and in particular copper pidolate such as Cuivridone® from Solabia;
extracts of plants of the species Arnica montana, Cinchona succirubra, Eugenia caryophyllata, Humulus lupulus, Hypericum perforatum, Mentha piperita, Rosmarinus officinalis, Salvia oficinalis and Thymus vulgaris, all sold, for example, by the company Maruzen;
extracts of meadowsweet (Spiraea ulmaria), such as the product sold under the name Sebonormine® by the company Silab;
extracts of the alga Laminaria saccharina, such as the product sold under the name Phlorogine® by the company Biotechmarine;
mixtures of extracts of salad burnet root (Sanguisorba officinalis/Poterium officinale), of ginger rhizomes (Zingiber officinalis) and of cinnamon bark (Cinnamomum cassia), such as the product sold under the name Sebustop® by the company Solabia;
linseed extracts, such as the product sold under the name Linumine® by the company Lucas Meyer;
Phellodendron extracts, such as those sold under the name Phellodendron extract BG by the company Maruzen or Oubaku liquid B by the company Ichimaru Pharcos;
mixtures of argan oil, of Serenoa serrulata (saw palmetto) extract and of sesame seed extract, such as the product sold under the name Regu SEB® by the company Pentapharm;
mixtures of extracts of willowherb, of Terminalia chebula, of nasturtium and of bioavailable zinc (microalgae), such as the product sold under the name Seborilys® by the company Green Tech;
extracts of Pygeum afrianum, such as the product sold under the name Pygeum afrianum sterolic lipid extract by the company Euromed;
extracts of Serenoa serrulata, such as the products sold under the name Viapure Sabal by the company Actives International or those sold by the company Euromed;
mixtures of extracts of plantain, of Berberis aquifolium and of sodium salicylate, such as the product sold under the name Seboclear® by the company Rahn;
clove extract, such as the product sold under the name Clove extract powder by the company Maruzen;
argan oil, such as the product sold under the name Lipofructyl® by Laboratoires Serobiologiques;
lactic protein filtrates, such as the product sold under the name Normaseb® by the company Sederma;
extracts of the alga Laminaria, such as the product sold under the name Laminarghane® by the company Biotechmarine;
oligosaccharides of the alga Laminaria digitata, such as the product sold under the name Phycosaccharide AC by the company Codif;
sugar cane extracts, such as the product sold under the name Policosonol® by the company Sabinsa;
sulfonated shale oil, such as the product sold under the name Ichthyol Pale® by the company Ichthyol;
European meadowsweet (Spiraea ulmaria) extracts, such as the product sold under the name Cytobiol® Ulmaire by the company Libiol;
sebacic acid, especially sold in the form of a sodium polyacrylate gel under the name Sebosoft® by the company Sederma;
glucomannans extracted from konjac tuber and modified with alkylsulfonate chains, such as the product sold under the name Biopol Beta by the company Arch Chemical;
extracts of Sophora angustifolia, such as those sold under the name Sophora powder or Sophora extract by the company Bioland;
extracts of Cinchona succirubra bark, such as the product sold under the name Red Bark HS by the company Alban Muller;
extracts of Quillaja saponaria, such as the product sold under the name Panama wood HS by the company Alban Muller;
glycine grafted onto an undecylenic chain, such as the product sold under the name Lipacide UG OR by the company SEPPIC;
the mixture of oleanolic acid and of nordihydroguaiaretic acid, such as the product sold in the form of a gel under the name AC.Net by the company Sederma;
phthalimidoperoxyhexanoic acid or phthalimidoperoxycaproic acid, also known as 6-(phthalimidoperoxy)hexanoic acid, ε-(phthalimidoperoxy)hexanoic acid or PAP; PAP is especially available in two commercial forms from the company Solvay.

Thus, the phthalimidoperoxycaproic acid may be used together with cyclodextrins, so as to improve the stability of the compound. Document EP 895 777 more particularly describes the formation of an adduct of ε-phthalimidoperoxyhexanoic acid with cyclodextrin. Such an encapsulated product is sold by the company Solvay under the trade name Eureco® HC-P11 (powdered inclusion complex of phthalimidoperoxycaproic acid with pharma-grade β-cyclodextrin).

Phthalimidoperoxycaproic acid is also provided in the form of an aqueous dispersion (containing 17% active material) under the trade name Eureco® HC L17. Phthalimidoperoxycaproic acid is also provided in the form of a powder under the trade name Eureco® HC-P11. Such aqueous dispersions of phthalimidoperoxycaproic acid are described in document EP 1 074 607;
tri(C₁₂-C₁₃)alkyl citrate sold under the name Cosmacol® ECI by the company Sasol; tri(C₁₄-C₁₅)alkyl citrate sold under the name Cosmacol® ECL by the company Sasol;
10-hydroxydecanoic acid, and especially mixtures of 10-hydroxydecanoic acid, of sebacic acid and of 1,10-decanediol, such as the product sold under the name Acnacidol® BG by the company Vincience; and
mixtures thereof.

Preferred anti-seborrhoeic active agents that may be mentioned include:
benzoyl peroxide and vitamin B6 (or pyridoxine),
zinc salts such as zinc gluconate, zinc pyrrolidonecarboxylate (or zinc pidolate), zinc lactate, zinc aspartate, zinc carboxylate, zinc salicylate and zinc cysteate;
copper derivatives and in particular copper pidolate such as Cuivridone® from Solabia;
meadowsweet (Spiraea ulmaria) extracts, such as the product sold under the name Sebonormine® by the company Silab;
extracts of the alga Laminaria saccharina, such as the product sold under the name Phlorogine® by the company Biotechmarine;
mixtures of extracts of salad burnet root (Sanguisorba officinalis/Poterium officinale), of ginger rhizomes (Zingiber officinalis) and of cinnamon bark (Cinnamomum cassia), such as the product sold under the name Sebustop® by the company Solabia;
clove extract, such as the product sold under the name Clove extract powder by the company Maruzen;
lactic protein filtrates, such as the product sold under the name Normaseb® by the company Sederma;
European meadowsweet (Spiraea ulmaria) extracts, such as the product sold under the name Cytobiol® Ulmaire by the company Libiol;
sebacic acid, especially sold in the form of a sodium polyacrylate gel under the name Sebosoft® by the company Sederma;
glycine grafted onto an undecylenic chain, such as the product sold under the name Lipacide UG OR by the company SEPPIC;
phthalimidoperoxyhexanoic acid or phthalimidoperoxycaproic acid (PAP);
tri(C₁₂-C₁₃)alkyl citrate sold under the name Cosmacol® ECI by the company Sasol; tri(C₁₄-C₁₅)alkyl citrate sold under the name Cosmacol® ECL by the company Sasol;
10-hydroxydecanoic acid, and especially mixtures of 10-hydroxydecanoic acid, of sebacic acid and of 1,10-decanediol, such as the product sold under the name Acnacidol® BG by the company Vincience; and
mixtures thereof.

Preferentially, the anti-seborrhoeic active agent is chosen from:
zinc salts such as zinc gluconate, zinc pyrrolidonecarboxylate (or zinc pidolate), zinc lactate, zinc aspartate, zinc carboxylate, zinc salicylate and zinc cysteate; and preferably zinc pyrrolidonecarboxylate (or zinc pidolate) or zinc salicylate;
copper derivatives and in particular copper pidolate such as Cuivridone® from Solabia;
phthalimidoperoxyhexanoic acid or phthalimidoperoxycaproic acid (PAP);
clove extract, such as the product sold under the name Clove extract powder by the company Maruzen;
glycine grafted onto an undecylenic chain, such as the product sold under the name Lipacide UG OR by the company SEPPIC;
tri(C₁₂-C₁₃)alkyl citrate sold under the name Cosmacol® ECI by the company Sasol; tri(C₁₄-C₁₅)alkyl citrate sold under the name Cosmacol® ECL by the company Sasol;
and mixtures thereof.

According to one preferred mode, phthalimidoperoxyhexanoic acid or phthalimidoperoxycaproic acid (PAP) is used.

According to another particular mode, a zinc salt is used, such as zinc gluconate, zinc pyrrolidone-carboxylate (or zinc pidolate), zinc lactate, zinc aspartate, zinc carboxylate, zinc salicylate or zinc cysteate; and preferably zinc pyrrolidonecarboxylate (or zinc pidolate) or zinc salicylate.

The anti-seborrhoeic active agent is, for example, present in a content ranging for example from 0.1% to 10% by weight, preferably from 0.1% to 5% by weight and preferentially from 0.5% to 3% by weight relative to the total weight of the composition.

### 21. Astringents

According to the invention, the term “astringents” means agents for combating the dilation of the sebaceous follicles.

As astringents that may be used in the composition according to the invention, mention may be made of extracts of mushroom pulp (Polyporus officinalis), for instance Laricyl LS8865® from Cognis, extracts of Terminalia catappa and Sambucus nigra, for instance Phytofirm LS9120® from Cognis, extracts of gall nut, for instance Tanlex VE® from Ichimaru Pharcos, aluminium hydroxychloride, centella extracts (e.g. Plantactiv centella from Cognis), dicetyl dimethylammonium chloride, for instance Varisoft 432 CG® from Degussa, common horsechestnut extracts, mallow extracts, witch-hazel extracts, sweet almond extracts, marshmallow root extracts and linseed extracts, for instance Almondermin LS 3380® from Cognis, burdock extracts, nettle extracts, birch extracts, horsetail extracts, camomile extracts, for instance those sold under the name Extrapone 9 Special® by the company Symrise, skullcap extracts, European meadowsweet extracts (for example Cytobiol Ulmaire from Libiol), a mixture of extracts of white ginger, of horsetail, of nettle, of rosemary and of yucca, for instance Herb extract B1348® from Bell Flavors & Fragrances, extracts of acacia, of elm, of white willow, of cinnamon, of birch and of meadowsweet, Panama sapogenins, zinc phenolsulfonate from Interchemical, extracts of gentian, of cucumber and of walnut, the mixture of extracts of Ratanhia, of grapefruit, of gumweed and of oak gall, for instance Epilami® from Alban Muller.

As preferred astringents according to the invention, use will be made of skullcap extracts, European meadowsweet extracts, meadowsweet extracts, gentian extracts, burdock extracts and cinnamon extracts, and mixtures thereof.

According to one preferred mode, an extract of cinnamon will be used.

### 22. Cicatrizing Agents

Examples of cicatrizing agents that may especially be mentioned include:

allantoin, urea, certain amino acids, for instance hydroxyproline, arginine, and serine, and also extracts of white lily (for instance Phytélène Lys 37EG 16295 from Indena), a yeast extract, for instance the cicatrizing agent LS LO/7225B from Laboratoires Serobiologiques), tamanu oil, extract of Saccharomyces cerevisiae, for instance Biodynes® TRF® from Arch Chemical, oat extracts, chitosan and derivatives, for instance chitosan glutamate, carrot extracts, artemia extract, for instance GP4G® from Vincience, sodium acexamate, lavandin extracts, propolis extracts, ximeninic acid and salts thereof, rose hip oil, marigold extracts, for instance Souci Ami® Liposolible from Alban Muller, horsetail extracts, lemon peel extracts, for instance Herbasol® citron from Cosmetochem, helichrysum extracts, common yarrow extracts and folic acid.

As preferred cicatrizing agents according to the invention, use will be made of arginine, serine, folic acid, tamanu oil, sodium acexamate, horsetail extracts and helichrysum extracts, and mixtures thereof.

### 23. Anti-Inflammatory Agents

As particular anti-inflammatory agents that may be used according to the invention, mention may be made of cortisone, hydrocortisone, indomethacin, betamethasone, azelaic acid, acetaminophen, diclofenac, clobetasol propionate, folic acid; an extract of Eperua falcata bark, such as the product sold by the company Cognis under the name Eperuline®; an extract of Paeonia suffruticosa root, such as the product sold by the company Ichimaru Pharcos under the name Botanpi Liquid B®; and mixtures thereof.

Preferred anti-inflammatory agents that will be mentioned are azelaic acid, folic acid, an extract of Eperua falcata bark, such as the product sold by the company Cognis under the name Eperuline®; an extract of Paeonia suffruticosa root, such as the product sold by the company Ichimaru Pharcos under the name Botanpi Liquid B®; and mixtures thereof.

According to one preferred mode, an extract of Eperua falcata bark will be used, such as the product sold by the company Cognis under the name Eperuline®.

### 24. Antiacne Agents

In one advantageous aspect of the invention, the composition may also comprise at least one anti-acne active agent.

The term “anti-acne active agent” especially means any active agent that has effects on the specific flora of greasy skin, for instance Propionibacterium acnes (P. acnes). These effects may be bactericidal.

Antibactericidal active agents that may especially be mentioned include:
active agents and preserving agents with antimicrobial activity mentioned in patent application DE 103 24 567, which is incorporated into the present invention by reference,
Asiatic acid,
the monoethanolamine salt of 1-hydroxy-4-methyl 6-trimethylpentyl-2-pyridone (INCI name: piroctone olamine), sold especially under the name Octopirox® by the company Clariant;
citronellic acid, perillic acid (or 4-isopropenylcyclohex-1-enecarboxylic acid),
glyceryl 2-ethylhexyl ether (INCI name: ethylhexylglycerine), for example sold under the name Sensiva SC 50® by the company Shulke & Mayr,
glyceryl caprylate/caprate, for example sold under the name Capmul MCM® by the company Abitec;
sodium calcium phosphosilicate, especially sold under the names Bioactive Glasspowder® and Actysse Premier BG® by the company Schott Glass;
silver-based particles, for example those sold under the name Metashine ME 2025 PS® by the company Nippon Sheet Glass;
hop cone extract (Humulus lupulus) obtained by supercritical CO₂ extraction, such as the product sold under the name HOP CO2-TO Extract® by the company Flavex Naturextrakte,
St.-John's Wort extract obtained by supercritical CO₂ extraction, such as the product sold under the name St.-John's Wort CO2-TO Extract® by the company Flavex Naturextrakte,
the mixture of extracts of roots of Scutellaria baicalensis, of Paeonia suffruticosa and Glycyrrhiza glabra, such as the product sold under the name BMB—CF® by the company Naturogin,
argan tree extract, for instance Argapure LS9710® from Cognis;
bearberry leaf extracts, for instance the product sold under the name Melfade-J by the company Pentapharm;
10-hydroxy-2-decanoic acid such as Acnacidol P® from Vincience, sodium ursolate, azelaic acid, diiodomethyl p-tolyl sulfone such as Amical Flowable® from Angus, malachite powder, zinc oxide such as Zincare® from Elementis GMBH, octadecenedioic acid such as Arlatone dioic DCA® from Uniqema; ellagic acid; 2,4,4′-trichloro-2′-hydroxydiphenyl ether (or triclosan), 1-(3′,4′-dichlorophenyl)-3-(4′-chlorophenyl)urea (or triclocarban), 3,4,4′-trichlorocarbanilide, 3′,4′,5′-trichlorosalicylanilide, phenoxyethanol, phenoxypropanol, phenoxyisopropanol, hexamidine isethionate, metronidazole and salts thereof, miconazole and salts thereof, itraconazole, terconazole, econazole, ketoconazole, saperconazole, fluconazole, clotrimazole, butoconazole, oxiconazole, sulfaconazole, sulconazole, terbinafine, ciclopirox, ciclopiroxolamine, undecylenic acid and salts thereof, benzoyl peroxide, 3-hydroxybenzoic acid, 4-hydroxybenzoic acid, phytic acid, N-acetyl-L-cysteine, lipoic acid, azelaic acid and salts thereof, arachidonic acid, resorcinol, 3,4,4′-trichlorocarbanalide, octoxyglycerine or octoglycerine, octanoylglycine such as Lipacid C8G® from SEPPIC, caprylyl glycol, 10-hydroxy-2-decanoic acid, dichlorophenylimidazoldioxolane and derivatives thereof described in patent application WO 93/18743, iodopropynyl butylcarbamate, 3,7,11-trimethyldodeca-2,5,10-trienol or farnesol, phytosphingosines; quaternary ammonium salts, for instance cetyltrimethylammonium salts and cetylpyridinium salts, and
mixtures thereof.

Mention may also be made of certain surfactants with an antimicrobial effect, for instance sodium cocoamphoacetate or disodium diacetate such as Miranol C2M Conc. NP, betaines, for instance the cocoyl betaine Genagen KB from Clariant, sodium lauryl ether sulfate, for instance Emal 270 D from Kao, decyl glucoside, for instance Plantacare 2000 UP, branched C12-13 dialkyl malates, for instance Cosmacol EMI, propylene glycol monoesters, for instance propylene glycol monolaurate, monocaprylate or monocaprate, lauryldimethylamine betaine, for instance Empigen BB/LS, and also polyquaternary ammoniums such as Quaternium-24 or Bardac 2050 from Lonza and those described in patent FR 0 108 283, and mixtures thereof.

As preferred antimicrobial agents, an agent chosen from caprylyl glycol, octoglycerine or octoxyglycerine, and 10-hydroxy-2-decanoic acid, and mixtures thereof, will be used in the compositions of the invention.

Other additional anti-acne active agents may be added to the abovementioned anti-acne active agents.

Mention may be made especially of active agents with bacterial anti-adhesion effects or agents that act on the biofilm of bacteria to prevent them from multiplying.

As agents for preventing and/or reducing the adhesion of microorganisms, mention may be made especially of: phytanetriol and derivatives thereof as described in patent application EP 1 529 523, plant oils such as wheatgerm oil, calendula oil, castor oil, olive oil, avocado oil, sweet almond oil, groundnut oil, jojoba oil, sesame seed oil, apricot kernel oil, sunflower oil and macadamia oil, described in patent EP 1 133 979, or certain surfactants such as disodium cocoamphodiacetate, oxyethylenated (7 EO) glyceryl cocoate, 18-hexadecenyl succinate, octoxyglyceryl palmitate, octoxyglyceryl behenate, dioctyl adipate, PPG-15 stearyl ether, and the branched C₁₂-C₁₃ dialkyl tartrates described in patent EP 1 129 694, and mixtures thereof.

In particular with regard to the propagation of P. acnes, or as active agents that act on the biofilm of bacteria to prevent them from proliferating, mention may be made of pentylene glycol, Nylon-66 (polyamide 66 fibres), rice bran oil, polyvinyl alcohol such as Celvol 540 PV Alcohol® from Celanese Chemical, rapeseed oil such as Akorex L® from Karlshamns, and fructose derivatives, and mixtures thereof.

The anti-acne active agent may be present in a content ranging for example from 0.01% to 10% by weight and preferably from 0.05% to 5% by weight relative to the total weight of the composition.

As a function of the nature and/or solubility of the abovementioned active agents, a person skilled in the art will know how to select the most suitable embodiment according to the invention.

As lipophilic active agents that may be used in the composition of the invention, mention may be made especially of D-α-tocopherol, DL-α-tocopherol, D-α-tocopheryl acetate, DL-α-tocopheryl acetate, ascorbyl palmitate, vitamin F glycerides, D vitamins, vitamin D2, vitamin D3, retinol, retinal esters, retinyl palmitate, retinyl propionate, carotenes including β-carotene, D-panthenol, farnesol, farnesyl acetate, salicylic acid and derivatives thereof, for instance 5-n-octanoylsalicylic acid, α-hydroxy acid alkyl esters such as citric acid, lactic acid, glycolic acid, asiatic acid, madecassic acid, asiaticoside, the total extract of Centella asiatica, β-glycyrrhetinic acid, α-bisabolol, ceramides, for instance 2-oleoylamino-1,3-octadecane, phytanetriol, phospholipids of marine origin rich in polyunsaturated essential fatty acids, ethoxyquine, rosemary extract, balm extract, quercetin, extract of dried microalgae, essential oil of bergamot, octyl methoxycinnamate, butylmethoxydibenzoylmethane, octyl triazone, 3,5-di-tert-butyl-4-hydroxy-3-benzylidenecamphor, antibiotics, antifungal agents, anaesthetics, analgesics, antiseptics, antiviral agents, pesticides and herbicides, and mixtures thereof.

The cosmetic and/or dermatological active agents will be present in the composition according to the invention in a content ranging for example from 0.001% to 20% relative to the total weight of the composition, preferably from 0.01% to 10%, even more preferentially from 0.5% to 5% and more preferably from 0.1% to 1% by weight relative to the total weight of the composition.

For “scrubbing” applications, the contents of cosmetic and/or dermatological active agents may range from 1% to 50% by weight relative to the total weight of the composition and preferably from 1% to 30% by weight relative to the total weight of the composition.

Scrubbing is a well-known means for improving the appearance and/or texture of the skin and/or the scalp, especially for improving the radiance and homogeneity of the complexion and/or for reducing the visible and/or tactile irregularities of the skin, and in particular for improving the surface appearance of the skin, for attenuating actinic lentigo, acne or chicken pox marks, and also for preventing, attenuating or combating the signs of ageing of the skin, and especially for smoothing out irregularities in the texture of the skin, such as wrinkles and fine lines.

It has the effect of removing a surface part of the skin to be treated (epidermis and possibly the upper layer of the dermis), via chemical methods.

Other Additional Ingredients

To complement and/or optimize the effects imparted by the cosmetic and/or dermatological active agents mentioned above on the keratin materials, it may be advantageous to incorporate into the compositions of the invention other additional ingredients.

In particular, these additional ingredients may impart an immediate visual effect that will be relayed by the biological effect of the active agents mentioned above.

They may also, via a mechanical action (e.g.: abrasive fillers), amplify the effect of the biological active agents mentioned above.

Thus, the composition according to the invention may also comprise at least one agent chosen from matting agents, fillers with a soft-focus effect, fluorescers, agents for promoting the naturally pinkish coloration of the skin, abrasive fillers or exfoliants, and mixtures thereof.

### Matting Agents

The term “matting agent” means agents intended to make the skin visibly more matt and less shiny.

The matting effect of the agent and/or composition containing it may especially be evaluated using a gonioreflectometer, by measuring the ratio R between the specular reflection and the scattered reflection. A value of R of less than or equal to 2 generally reflects a matting effect.

The matting agent may especially be chosen from a rice starch or a corn starch, kaolinite, talc, a pumpkin seed extract, cellulose microbeads, plant fibres, synthetic fibres, in particular polyamide fibres, expanded acrylic copolymer microspheres, polyamide powders, silica powders, polytetrafluoroethylene powders, silicone resin powders, acrylic polymer powders, wax powders, polyethylene powders, powders of elastomeric crosslinked organopolysiloxane coated with silicone resin, talc/titanium dioxide/alumina/silica composite powders, amorphous mixed silicate powders, silicate particles and especially mixed silicate particles, and mixtures thereof.

Examples of matting agents that may especially be mentioned include:
rice or corn starch, in particular an aluminium starch octenyl succinate sold under the name Dry Flo® by the company National Starch;
kaolinite;
silicas;
talc;
a pumpkin seed extract as sold under the name Curbilene® by the company Indena;
cellulose microbeads as described in patent application EP 1 562 562;
fibres, such as silk fibre, cotton fibre, wool fibre, flax fibre, cellulose fibre extracted especially from wood, from vegetables or from algae, polyamide fibre (Nylon®), modified cellulose fibre, poly-p-phenyleneterephthamide fibre, acrylic fibre, polyolefin fibre, glass fibre, silica fibre, aramid fibre, carbon fibre, Teflon® fibre, insoluble collagen fibre, polyester fibre, polyvinyl chloride or polyvinylidene chloride fibre, polyvinyl alcohol fibre, polyacrylonitrile fibre, chitosan fibre, polyurethane fibre, polyethylene phthalate fibre, fibres formed from a mixture of polymers, resorbable synthetic fibres, and mixtures thereof described in patent application EP 1 151 742;
expanded acrylic copolymer microspheres such as those sold by the company EXPANCEL under the name Expancel 551®;
fillers with an optical effect as described in patent application FR 2 869 796, in particular:
   polyamide powders (Nylon®), for instance Nylon 12 particles of the Orgasol type from Arkema, with a mean size of 10 microns and a refractive index of 1.54,
   silica powders, for instance Silica beads SB150 from Miyoshi with a mean size of 5 microns and a refractive index of 1.45,
   polytetrafluoroethylene powders, for instance PTFE Ceridust 9205F from Clariant, with a mean size of 8 microns and a refractive index of 1.36,
   silicone resin powders, for instance the silicone resin Tospearl 145A from GE Silicone with a mean size of 4.5 microns and a refractive index of 1.41,
   acrylic copolymer powders, especially of polymethyl(meth)acrylate, for instance the PMMA particles Jurymer MBI from Nihon Junyoki, with a mean size of 8 microns and a refractive index of 1.49, or the Micropearl M100® and F 80 ED® particles from the company Matsumoto Yushi-Seiyaku,
   wax powders, for instance the paraffin wax particles Microease 114S from Micropowders, with a mean size of 7 microns and a refractive index of 1.54,
   polyethylene powders, especially comprising at least one ethylene/acrylic acid copolymer, and in particular consisting of ethylene/acrylic acid copolymers, for instance the particles Flobeads EA 209 from Sumitomo (with a mean size of 10 microns and a refractive index of 1.48),
   elastomeric crosslinked organopolysiloxane powders coated with silicone resin, especially with silsesquioxane resin, as described, for example, in U.S. Pat. No. 5,538,793. Such elastomeric powders are sold under the names KSP-100, KSP-101, KSP-102, KSP-103, KSP-104 and KSP-105 by the company Shin-Etsu, and
   talc/titanium dioxide/alumina/silica composite powders such as those sold under the name Coverleaf® AR-80 by the company Catalyst & Chemicals,
   mixtures thereof,
   compounds that absorb and/or adsorb sebum as described in patent application FR 2 869 796. Mention may be made especially of:
   silica powders, for instance the porous silica microspheres sold under the name Silica Beads SB-700 sold by the company Myoshi, the products Sunsphere® H51, Sunsphere® H33 and Sunsphere® H53 sold by the company Asahi Glass; the polydimethylsiloxane-coated amorphous silica microspheres sold under the name SA Sunsphere® H-33 and SA Sunsphere® H-53 sold by the company Asahi Glass;
   amorphous mixed silicate powders, especially of aluminium and magnesium, for instance the product sold under the name Neusilin UFL2 by the company Sumitomo;
   polyamide (Nylon®) powders, for instance Orgasol® 4000 sold by the company Arkema, and
   acrylic polymer powders, especially of polymethyl methacrylate, for instance Covabead® LH85 sold by the company Wackherr; of polymethyl methacrylate/ethylene glycol dimethacrylate, for instance Dow Corning 5640 Microsponge® Skin Oil Adsorber sold by the company Dow Corning, or Ganzpearl® GMP-0820 sold by the company Ganz Chemical; of polyallyl methacrylate/ethylene glycol dimethacrylate, for instance Poly-Pore® L200 or Poly-Pore® E200 sold by the company Amcol; of ethylene glycol dimethacrylate/lauryl methacrylate copolymer, for instance Polytrap® 6603 sold by the company Dow Corning;
   silicate particles, such as alumina silicate;
   mixed silicate particles, such as:
   magnesium aluminium silicate particles, such as saponite or hydrated magnesium aluminium silicate with a sodium sulfate sold under the trade name Sumecton® by the company Kunimine;
   the magnesium silicate,
   hydroxyethylcellulose, black cumin oil, marrow oil and phospholipids complex or Matipure® from Lucas Meyer, and
   mixtures thereof.

Preferred matting agents that may be used according to the invention include a pumpkin seed extract, a rice or corn starch, kaolinite, silicas, talc, polyamide powders, polyethylene powders, acrylic copolymer powders, expanded acrylic copolymer microspheres, silicone resin microbeads and mixed silicate particles, and mixtures thereof.

Fillers with a Soft-Focus Effect

These fillers may be any material capable of modifying and hiding wrinkles by virtue of their intrinsic physical properties. These fillers may especially modify wrinkles via a tensioning effect, a covering effect or a soft-focus effect.

Examples of fillers that may be given include the following compounds:
porous silica microparticles, for instance the silica beads SB150 and SB700 from Miyoshi with a mean size of 5 μm; the series-H Sunspheres from Asahi Glass, for instance Sunspheres H33, H51 with respective sizes of 3.5 and 5 μm;
hollow hemispherical silicone resin particles such as NLK 500®, NLK 506® and NLK 510® from Takemoto Oil and Fat, especially described in EP-A-1 579 849;
silicone resin powders, for instance the silicone resin Tospearl® 145A from GE Silicone, with a mean size of 4.5 μm;
acrylic copolymer powders, especially of polymethyl (meth)acrylate, for instance the PMMA particles Jurymer MBI® from Nihon Junyoki, with a mean size of 8 μm, the hollow PMMA spheres sold under the name Covabead® LH85 by the company Wackherr, and vinylidene/acrylonitrile/methylene methacrylate expanded microspheres sold under the name Expancel®;
wax powders, for instance the paraffin wax particles MicroEase® 114S from MicroPowders, with a mean size of 7 μm;
polyethylene powders, especially comprising at least one ethylene/acrylic acid copolymer, and in particular consisting of ethylene/acrylic acid copolymers, for instance the Flobeads® EA 209 particles from Sumitomo, with a mean size of 10 μm;
crosslinked elastomeric organopolysiloxane powders coated with silicone resin and especially with silsesquioxane resin, under the names KSP-100®, KSP-101®, KSP-102®, KSP-103®, KSP-104® and KSP-105® by the company Shin-Etsu;
talc/titanium dioxide/alumina/silica composite powders, for instance those sold under the name Coverleaf AR-80® by the company Catalyst & Chemicals;
talc, mica, kaolin, lauryl glycine, starch powders crosslinked with octenyl succinate anhydride, boron nitride, polytetrafluoroethylene powders, precipitated calcium carbonate, magnesium carbonate, magnesium hydrogen carbonate, barium sulfate, hydroxyapatite, calcium silicate, cerium dioxide and glass or ceramic microcapsules;
hydrophilic or hydrophobic, synthetic or natural, mineral or organic fillers such as silk fibres, cotton fibres, wool fibres, flax fibres, cellulose fibres extracted especially from wood, vegetables or algae, Polyamides (Nylon®) fibres, modified cellulose fibres, poly-p-terephthamide fibres, acrylic fibres, polyolefin fibres, glass fibres, silica fibres, aramid fibres, carbon fibres, polytetrafluoroethylene (Teflon®) fibres, insoluble collagen fibres, polyester fibres, polyvinyl chloride fibres, polyvinylidene chloride fibres, polyvinyl alcohol fibres, polyacrylonitriles fibres, chitosan fibres, polyurethane fibres, polyethylene phthalate fibres, fibres formed from a mixture of polymers, resorbable synthetic fibres, and mixtures thereof described in patent application EP 1 151 742;
spherical elastomeric crosslinked silicones, for instance Trefil E-505C® or E-506C® from Dow Corning;
abrasive fillers, which, via a mechanical effect, smooth out the skin microrelief, such as abrasive silica, for instance Abrasif SP® from Semanez or nutshell powders (for example of apricot or walnut, from Cosmetochem).

The fillers with an effect on the signs of ageing are especially chosen from porous silica microparticles, hollow hemispherical silicones, silicone resin powders, acrylic copolymer powders, polyethylene powders, crosslinked elastomeric organopolysiloxanes powders coated with silicone resin, talc/titanium dioxide/alumina/silica composite powders, precipitated calcium carbonate, magnesium carbonate, magnesium hydrogen carbonate, barium sulfate, hydroxyapatite, calcium silicate, cerium dioxide, glass or ceramic microcapsules, and silk fibres or cotton fibres, and mixtures thereof.

The filler may be a soft-focus filler.

The term “soft-focus” filler means a filler which in addition gives the complexion transparency and a hazy effect. Preferably, the soft-focus fillers have a mean particle size of less than or equal to 15 microns. These particles may be in any form and in particular may be spherical or non-spherical. These fillers are more preferably non-spherical.

The soft-focus fillers may be chosen from silica and silicate powders, especially alumina powder, powders of polymethyl methacrylate (PMMA) type, talc, silica/TiO₂ or silica/zinc oxide composites, polyethylene powders, starch powders, polyamide powders, styrene/acrylic copolymer powders and silicone elastomers, and mixtures thereof.

Mention may be made in particular of talc with a number-average size of less than or equal to 3 microns, for example talc with a number-average size of 1.8 microns and especially the product sold under the trade name Talc P3® by the company Nippon Talc, Nylon® 12 powder, especially the product sold under the name Orgasol 2002 Extra D Nat Cos® by the company Atochem, silica particles 1% to 2% surface-treated with a mineral wax (INCI name: hydrated silica (and) paraffin) such as the products sold by the company Degussa, amorphous silica microspheres, such as the products sold under the name Sunsphere, for example of reference H-53® by the company Asahi Glass, and silica microbeads such as those sold under the name SB-700® or SB-150® by the company Miyoshi, this list not being limiting.

The concentration of these fillers with an effect on the signs of ageing in the compositions according to the invention may be between 0.1% and 40%, or even between 0.1% and 20% by weight, relative to the total weight of the composition.

### Fluorescers

The term “fluorescer” means a substance which, under the effect of ultraviolet rays and/or visible light, re-emits in the visible region the portion of light that it has absorbed under the same colour as that which it naturally reflects. The naturally reflected colour is thus reinforced by the re-emitted colour and appears extremely bright.

Examples that may be mentioned include coloured polyamide and/or formaldehyde/benzoguanamine and/or melamine/formaldehyde/sulfonamide resins, from coloured aminotriazine/formaldehyde/sulfonamide co-condensates and/or from metallized polyester flakes and/or mixtures thereof. These fluorescent pigments may also be present in the form of aqueous dispersions of fluorescent pigments.

Mention may also be made of the pink-coloured fluorescent aminotriazine/formaldehyde/sulfonamide co-condensate with a mean particle size of 3-4 microns sold under the trade name “Fiesta Astral Pink FEX-1” and the blue-coloured fluorescent aminotriazine/formaldehyde/sulfonamide co-condensate with a mean particle size of 3-4.5 microns sold under the trade name “Fiesta Comet Blue FTX-60” by the company Swada, or alternatively the yellow-coloured benzoguanamine/formaldehyde resin covered with formaldehyde/urea resin sold under the trade name “FB-205 Yellow” and the red-coloured benzoguanamine/formaldehyde resin covered with formaldehyde/urea resin sold under the trade name “FB-400 Orange Red” by the company UK Seung Chemical, and the orange-coloured polyamide resin sold under the trade name “Flare 911 Orange 4” by the company Sterling Industrial Colors.

The fluorescent substances are preferably present in the composition in a content ranging for example from 0.1% to 20%, preferably from 0.1% to 15% and more preferably from 0.5% to 3% by weight relative to the total weight of the composition.

When the organic fluorescent substances are white, they are also known as optical brighteners.

The optical brightener has the effect of intensifying the radiance and reviving the shades of cosmetic compositions comprising them on application to the skin.

Among the optical brighteners that may be mentioned more particularly are stilbene derivatives, in particular polystyrylstilbenes and triazinestilbenes, coumarin derivatives, in particular hydroxycoumarins and aminocoumarins, oxazole, benzoxazole, imidazole, triazole and pyrazoline derivatives, pyrene derivatives and porphyrin derivatives, and/or mixtures thereof.

Such compounds are available, for example, under the trade names Tinopal SOP® and Uvitex OB® from the company Ciba Geigy.

The optical brighteners preferentially used are sodium 4,4′-bis[(4,6-dianilino-1,3,5-triazin-2-yl)amino]-stilbene-2,2′-disulfonate, 2,5-thiophenediylbis(5-tert-butyl-1,3-benzoxazole) and disodium 4,4′-distyrylbiphenylsulfonate, and/or mixtures thereof.

### Agents for Promoting the Naturally Pinkish Coloration of the Skin

Mention may be made especially of:
a self-tanning agent, i.e. an agent which, when applied to the skin, especially to the face, can produce a tan effect that is more or less similar in appearance to that which may result from prolonged exposure to the sun (natural tan) or under a UV lamp;
an additional colouring agent, i.e. any compound that has particular affinity for the skin, which allows it to give the skin a lasting, non-covering coloration (i.e. that does not have a tendency to opacify the skin) and that is not removed either with water or using a solvent, and that withstands both rubbing and washing with a solution containing surfactants. Such a lasting coloration is thus distinguished from the superficial and transient coloration provided, for example, by a makeup pigment;
   and mixtures thereof.

Examples of self-tanning agents that may especially be mentioned include:
dihydroxyacetone (DHA),
erythrulose, and
the combination of a catalytic system formed from:
manganese and/or zinc oxide salts, and
alkali metal and/or alkaline-earth metal hydrogen carbonates.

The self-tanning agents are generally chosen from monocarbonyl or polycarbonyl compounds, for instance isatin, alloxan, ninhydrin, glyceraldehyde, mesotartaric aldehyde, glutaraldehyde, erythrulose, pyrazoline-4,5-dione derivatives as described in patent application FR 2 466 492 and WO 97/35842, dihydroxyacetone (DHA) and 4,4-dihydroxypyrazolin-5-one derivatives as described in patent application EP 903 342. DHA will preferably be used.

The DHA may be used in free and/or encapsulated form, for example in lipid vesicles such as liposomes, especially described in patent application WO 97/25970.

In general, the self-tanning agent is present in an amount ranging for example from 0.01% to 20% by weight and preferably in an amount of between 0.1% and 10% of the total weight of the composition.

Examples of additional colouring agents that may be mentioned include plant extracts, for instance sorghum extracts obtained from the whole plant or from the stems, seeds or leaves of the genus Sorghum. The preferred Sorghum species are chosen from Sorghum bicolor, Sorghum caudatum, Sorghum nervosum, Sorghum durra, Sorghum vulgare and Sorghum species in combination with Colletotrichum graminicola, for instance those described in patent application FR 02/00251.

Other dyes that allow modification of the colour produced by the self-tanning agent may also be used.

These dyes may be chosen from synthetic or natural direct dyes.

These dyes may be chosen, for example, from red or orange dyes of the fluorane type such as those described in patent application FR 2 840 806. Mention may be made, for example, of the following dyes:
tetrabromofluoresceine or eosin known under the CTFA name: CI 45380 or Red 21;
phloxin B known under the CTFA name: CI 45410 or Red 27;
diiodofluoresceine known under the CTFA name: CI 45425 or Orange 10;
dibromofluoresceine known under the CTFA name: CI 45370 or Orange 5;
the sodium salt of tetrabromofluoresceine known under
the CTFA name: CI 45380 (Na salt) or Red 22;
the sodium salt of phloxin B known under the CTFA name: CI 45410 (Na salt) or Red 28;
the sodium salt of diiodofluoresceine known under the CTFA name: CI 45425 (Na salt) or Orange 11;
erythrosine known under the CTFA name: CI 45430 or Acid Red 51;
phloxin known under the CTFA name: CI 45405 or Acid Red 98.

These dyes may also be chosen from anthraquinones, caramel, carmine, carbon black, azulene blues, methoxalene, trioxalene, guajazulene, chamuzulene, Bengal rose, cosine 10B, cyanosin and daphinin.

These dyes may also be chosen from indole derivatives, for instance the monohydroxyindoles as described in patent FR 2 651 126 (i.e.: 4-, 5-, 6- or 7-hydroxyindole) or the dihydroxyindoles as described in patent EP-B-0 425 324 (i.e.: 5,6-dihydroxyindole, 2-methyl-5,6-dihydroxyindole, 3-methyl-5,6-dihydroxyindole or 2,3-dimethyl-5,6-dihydroxyindole).

### Abrasive Fillers or Exfoliants

As exfoliants that may be used in rinse-out compositions according to the invention, examples that may be mentioned include exfoliants for scrubbing particles of mineral, plant or organic origin. Thus, polyethylene beads or powder, Nylon powder, polyvinyl chloride powder, pumice powder, ground apricot kernel or walnut husk, sawdust, glass beads and alumina, and mixtures thereof, may be used, for example.

Mention may also be made of Exfogreen® from Solabia (bamboo extract), extracts of strawberry akenes (Strawberry Akenes from Greentech), peach kernel powder, apricot kernel powder, and finally, in the field of plant powders with an abrasive effect, mention may be made of cranberry kernel powder.

As abrasive fillers or exfoliants that are preferred according to the invention, mention will be made of peach kernel powder, apricot kernel powder, cranberry kernel powder, strawberry akene extracts and bamboo extracts.

The additional ingredients(s) used in the kit or one of the compositions according to the invention may represent from 0.0001% to 20%, preferably from 0.01% to 10% and better still from 0.01% to 10% by weight relative to the total weight of the composition.

According to one particular mode, the composition according to the invention contains at least urea or a derivative thereof, such as Hydrovance® from National Starch.

According to another mode, the composition according to the invention contains at least acrylic acid homopolymers, for instance Lipidure-HM® from NOF Corporation.

According to another mode, the composition according to the invention contains at least spheres of collagen and of chondroitin sulfate of marine origin (Atelocollagen) sold by the company Engelhard Lyon under the name marine filling spheres.

In particular, the combination between the phenanthrenol compound and the spheres of collagen and of chondroitin sulfate of marine origin and/or the composition containing it is used according to the invention for improving the moisturization of the skin.

According to another mode, the composition according to the invention contains at least hyaluronic acid or hyaluronic acid spheres.

In particular, the combination between the phenanthrenol compound and the hyaluronic acid or hyaluronic acid spheres and/or the composition containing it is used according to the invention for improving the moisturization of the skin.

According to another mode, the composition according to the invention contains at least kojic acid.

In particular, the combination between the phenanthrenol compound and kojic acid and/or the composition containing it is used according to the invention for improving the depigmentation of the skin.

According to another mode, the composition according to the invention contains at least ellagic acid.

In particular, the combination between the phenanthrenol compound and ellagic acid and/or the composition containing it is used according to the invention for improving the depigmentation of the skin.

According to another mode, the composition according to the invention contains at least 5-n-octanoylsalicylic acid, also known as capryloyl salicylic acid as the INCI name, optionally in combination with salicylic acid.

According to one particular mode, the 5-n-octanoylsalicylic acid is formulated in porous particles of polyamide fibres such as porous Nylon-12 particles described previously.

According to another mode, the composition according to the invention contains at least 8-hexadecene-1,16-dicarboxylic acid or 9-octadecenedioic acid and derivatives thereof.

According to another mode, the composition according to the invention contains at least 4-(2-hydroxyethyl)piperazine-1-propanesulfonic acid (HEPES).

According to another mode, the composition according to the invention contains at least 2-oxothiazolidine-4-carboxylic acid (procysteine) and derivatives thereof.

The according to another mode, the composition according to the invention contains at least {2-[acetyl(3-trifluoromethylphenyl)amino]-3-methylbutyrylamino}acetic acid, also known as N—[N-acetyl, N′-(3-trifluoromethyl)phenylvalyl]glycine, or N-acetyl-N-[3-(trifluoromethyl)phenyl]valylglycine or acetyl trifluoromethyl phenyl valylglycine.

According to another mode, the composition according to the invention contains at least one sphingosine, for instance salicyloyl sphingosine sold under the name Phytosphingosine® SLC by the company Degussa.

In particular, the combination between a phenanthrenol compound and at least one phytosphingosine and/or the composition containing it is used according to the invention for improving the biomechanical properties of the skin.

According to another mode, the composition according to the invention contains at least one ceramide or derivative, in particular ceramides of type 2 (for instance N-oleoyldihydrosphingosine, as the INCI name), of type 3 (for instance stearoyl-4-hydroxysphinganine, as the INCI name) and of type 5 (for instance N-2-hydroxypalmitoyldihydrosphingosine, having the INCI name: hydroxypalmitoyl sphinganine).

According to another mode, the composition according to the invention contains at least one ascorbyl glucoside (vitamin CG).

In particular, the combination between the phenanthrenol compound and ascorbyl glucoside and/or the composition containing it is used according to the invention for improving the depigmentation of the skin.

According to another mode, the composition according to the invention contains at least vitamin B3.

According to another mode, the composition according to the invention contains at least biotin.

According to another mode, the composition according to the invention contains at least caprylyl glycol. In particular, the combination between a phenanthrenol compound and at least caprylyl glycol and/or the composition containing it is used according to the invention for improving the moisturization of the skin.

According to another mode, the composition according to the invention contains at least one phytocomplex comprising cinnamic acid, phloroglucinol and a soybean extract. A soybean extract that will be used in particular is hydrolysed soybean protein, such as the product sold by Silab under the name Ridulisse®.

In particular, the combination between the phenanthrenol compound and the phytocomplex and/or the composition containing it is used according to the invention for preventing and/or treating hormonal ageing of the area around the lips, in particular the moustache area, and especially in the case of menopausal women.

According to another mode, the composition according to the invention contains at least phloretin.

In particular, the combination between a phenanthrenol compound and phloretin and/or the composition containing it is used according to the invention for improving the antioxidant effect of the composition.

According to another mode, the composition according to the invention contains at least one pomegranate extract.

In particular, the combination between a phenanthrenol compound and a pomegranate extract and/or the composition containing it is used according to the invention for improving the antioxidant effect of the composition.

According to another mode, the composition according to the invention contains at least ferulic acid.

In particular, the combination between a phenanthrenol compound and ferulic acid and/or the composition containing it is used according to the invention for improving the antioxidant effect of the composition.

According to another mode, the composition according to the invention contains at least adenosine.

In particular, the combination between a phenanthrenol compound and adenosine and/or the composition containing it is used according to the invention for improving the dermo-relaxation and/or dermo-contraction of the skin, and thus especially for preventing and/or treating facial expression wrinkles.

According to another mode, the composition according to the invention contains at least ginger.

In particular, the combination between a phenanthrenol compound and ginger and/or the composition containing it is used according to the invention for improving the dermo-relaxation and/or dermo-contraction of the skin, and thus especially for preventing and/or treating facial expression wrinkles.

According to another mode, the composition according to the invention contains at least manganese gluconate.

According to another mode, the composition according to the invention contains at least one sapogenin or a natural extract containing the same, in particular an extract of wild yam.

According to another mode, the composition according to the invention contains at least one extract of sea fennel.

In particular, the combination between the phenanthrenol compound and the extract of sea fennel and/or the composition containing it is used according to the invention for improving the synthesis of collagen, and thus for improving the biomechanical properties of the skin, in particular its firmness.

According to another mode, the composition according to the invention contains at least retinol or a derivative thereof, in particular retinyl palmitate.

In particular, the combination between the phenanthrenol compound and retinol or a derivative thereof and/or the composition containing it is used according to the invention for improving the synthesis of collagen, and thus for improving the biomechanical properties of the skin, in particular its firmness

According to another mode, the composition according to the invention contains at least one extract of blueberry (Vaccinium angustifolium or Vaccinium myrtillus).

In particular, the combination between the phenanthrenol compound and the extract of blueberry and/or the composition containing it is used according to the invention for promoting the synthesis of collagen and/or for preventing its degradation, and/or for promoting the stimulation and/or production of glycosaminoglycans.

According to another mode, the composition according to the invention contains at least one active agent for stimulating the synthesis of glycosaminoglycans, such as a C-glycoside derivative, in particular C-β-D-xylopyranoside-2-hydroxypropane or essence of Mamaku.

In particular, the combination between the phenanthrenol compound and the C-β-D-xylopyranoside-2-hydroxypropane or the essence of Mamaku and/or the composition containing it is used according to the invention for promoting the production of glycosaminoglycans.

According to another mode, the composition according to the invention contains at least one extract of black tea, for instance Kombuchka.

In particular, the combination between the phenanthrenol compound and the extract of black tea and/or the composition containing it is used according to the invention for promoting the cutaneous microcirculation, and thus especially for improving the appearance of the area around the eyes, in particular for reducing the shadows under the eyes.

According to another mode, the composition according to the invention contains at least one extract of black tea combined with at least one extract of brown sugar, one extract of lychee, or a mixture thereof.

In particular, the combination between the phenanthrenol compound and these three extracts and/or the composition containing it is used according to the invention for improving the appearance and/or visibility of the pores, especially by reducing the size and/or number of visible pores on facial skin, and in particular in the T area (forehead, nose, cheeks and chin), in particular in the region of the nose and the cheeks.

According to another mode, the composition according to the invention contains at least methylsilanol mannuronate.

According to another mode, the composition according to the invention contains at least folic acid or vitamin B9.

According to another mode, the composition according to the invention contains at least lycopene.

According to another mode, the composition according to the invention contains at least one yeast extract, in particular an extract of Saccharomyces cerevisiae.

According to another mode, the composition according to the invention contains at least one extract of lupin sold by the company Silab under the trade name Structurine®.

According to another mode, the composition according to the invention contains at least one extract of hydrolysed soybean protein.

According to another mode, the composition according to the invention contains at least rice protein or peptides.

According to one particular mode, the composition according to the invention contains at least arginine and/or serine.

In particular, the combination between a phenanthrenol compound and arginine or serine and/or the composition containing it is used according to the invention for improving and/or reinforcing the barrier function and/or for improving the cicatrization process.

According to another particular mode, the composition according to the invention contains at least omega-3 and/or omega-5 unsaturated oils such as musk rose oil.

In particular, the combination between a phenanthrenol compound and omega-3 and/or omega-5 unsaturated oils and/or the composition containing it is used according to the invention for improving the barrier function of the skin.

According to another particular mode, the composition according to the invention contains at least one extract of Artemisia abrotanum, such as Pulpactyl from Silab.

In particular, the combination between the phenanthrenol compound and the extract of Artemisia abrotanum and/or the composition containing it is used according to the invention for improving the lipostructure of the skin, and thus for reducing the age-related hollowing of the face.

According to another particular mode, the composition according to the invention contains at least one calmative chosen from glycyrrhetinic acid and salts and derivatives thereof, extract of Eperua falcata bark, and a mixture thereof.

According to another particular mode, the composition according to the invention contains at least one sebo-regulating agent chosen from zinc salts, copper derivatives, phthalimidoperoxyhexanoic acid and phthalimidoperoxycaproic acid (PAP), and mixtures thereof.

According to yet another mode, the composition according to the invention contains at least one astringent, in particular an extract of cinnamon.

According to one particular mode, the composition comprises, in a physiologically acceptable medium, at least one phenanthrenol compound, at least one desquamating agent and at least one calmative.

It may also comprise at least one foaming surfactant if it is a cleansing composition.

For a care composition intended for greasy skin, it will be advantageous to formulate (encapsulate) the phenanthrenol compound and/or the desquamating agent and/or the calmative in porous polyamide particles, in particular porous Nylon-12 particles to promote targeting of the follicles.

According to one preferred mode, the composition of the invention comprises a phenanthrenol compound, 5-n-octanoylsalicylic acid, salicylic acid, a potassium salt of glycyrrhetinic acid and an extract of Eperua falcata, the phenanthrenol compounds, 5-n-octanoylsalicylic acid and potassium salt of glycyrrhetinic acid being formulated (encapsulated) in porous polyamide particles, in particular porous Nylon-12 particles to promote targeting of the follicles.

According to another preferred mode, the composition according to the invention comprises a phenanthrenol compound, 5-n-octanoylsalicylic acid, salicylic acid, a potassium salt of glycyrrhetinic acid, an extract of Eperua falcata and at least one foaming surfactant.

A decyl glucoside, a sodium lauryl ether sulfate, an oxyethylenated (7 EO) glyceryl cocoate, and mixtures thereof, are used in particular as foaming surfactant.

According to another mode, the composition according to the invention contains at least one silica-alumina composite colloidal particle, for example those sold by the company Grace under the names Ludox AM, Ludox AM-X 6021, Ludox HSA and Ludox TMA.

According to another mode, the composition according to the invention contains at least one grafted silicone polymer such as those sold especially by the company 3M under the trade names VS 80, VS 70 or L021.

According to another mode, the composition according to the invention contains at least one extract of rye seed, such as the product sold by Silab under the name Coheliss®.

In particular, the combination between the phenanthrenol compound and the extract of rye seed and/or the composition is used according to the invention for improving the biomechanical properties of the skin, in particular its firmness and/or its elasticity.

According to another mode, the composition according to the invention contains at least caffeine.

According to another mode, the composition according to the invention contains at least ginseng.

According to another mode, the composition according to the invention contains at least ginkgo.

According to another mode, the composition according to the invention contains at least ruscus.

According to another mode, the composition according to the invention contains at least escin.

According to another mode, the composition according to the invention contains at least one extract of mint, in particular of mint leaves, for instance Calmiskin® from Silab.

In particular, the combination between the phenanthrenol compound and the extract of mint and/or the composition containing it is used according to the invention for soothing the skin.

According to another mode, the composition according to the invention contains at least one extract of filamentous bacteria, for instance Vitreoscilla filiformis as described in patent EP 761 204 and sold by Chimex under the name Mexoryl SBG®.

In particular, the combination between the phenanthrenol compound and the extract of Vitreoscilla filiformis and/or the composition containing it is used according to the invention for soothing the skin.

According to another mode, the composition according to the invention contains at least one extract of rose petals, for instance Rose Flower Herbasol® extract from the company Cosmetochem.

In particular, the combination between the phenanthrenol compound and the extract of rose and/or the composition containing it is used according to the invention for soothing the skin.

According to another mode, the composition according to the invention contains at least one fermented extract of Alteronomas sold under the name Abyssine®.

In particular, the combination between the phenanthrenol compound and the fermented extract of Alteronomas and/or the composition containing it is used according to the invention for soothing the skin.

According to another mode, the composition according to the invention contains at least one spring water from the Vichy basin, such as waters originating from the Célestins, Chomel, Grande-Grille, Hôpital, Lucas and Parc sources, and preferably water from the Lucas source.

In particular, the combination between the phenanthrenol compound and the spring water from the Vichy basin and/or the composition containing it is used according to the invention for soothing the skin.

According to another mode, the composition according to the invention contains at least one extract of linseed, such as the product sold under the name Linumine® by the company Lucas Meyer.

According to another mode, the kit or one of the compositions according to the invention contains at least one emulsifying silicone elastomer such as those sold under the names KSG-210, KSG-310, KSG-320, KSG-330, KSG-440, KSG-710, KSG-830 and KSG-840 by the company Shin-Etsu.

According to another mode, the composition according to the invention contains at least one silicone elastomer, such as an elastomeric organopolysiloxane, which is preferably at least partially crosslinked (e.g.: KSG).

According to another mode, the composition according to the invention contains at least DHA.

### Cosmetic Assembly

According to another aspect, the invention also relates to a cosmetic assembly comprising:
i) a container delimiting at least one compartment, the container being closed by means of a closing member; and
ii) a composition as described above, placed inside the compartment.

The container may be in any adequate form. It may especially be in the form of a bottle, a tube, a jar, a case, a box, a sachet or a carton.

The closing member may be in the form of a removable stopper, a lid, a cap, a tear-off strip or a capsule, especially of the type comprising a body attached to the container and a cover cap articulated on the body. It may also be in the form of a member for selectively closing the container, especially a pump, a valve or a flap valve.

The container may be combined with an applicator. The applicator may be in the form of a fine brush, as described, for example, in patent FR 2 722 380. The applicator may be in the form of a block of foam or of elastomer, a felt or a spatula. The applicator may be free (tuft or sponge) or securely fastened to a rod borne by the closing member, as described, for example, in U.S. Pat. No. 5,492,426. The applicator may be securely fastened to the container, as described, for example, in patent FR 2 761 959.

The product may be contained directly in the container, or indirectly. By way of example, the product may be arranged on an impregnated support, especially in the form of a wipe or a pad, and arranged (individually or in plurality) in a box or in a sachet. Such a support incorporating the product is described, for example, in patent application WO 01/03538.

The closing member may be coupled to the container by screwing. Alternatively, the coupling between the closing member and the container is done other than by screwing, especially via a bayonet mechanism, by click-fastening, gripping, welding, bonding or by magnetic attraction. The term “click-fastening” in particular means any system involving the crossing of a bead or cord of material by elastic deformation of a portion, especially of the closing member, followed by return to the elastically unconstrained position of the portion after the crossing of the bead or cord.

The container may be at least partially made of thermoplastic material. Examples of thermoplastic materials that may be mentioned include polypropylene or polyethylene.

Alternatively, the container is made of non-thermoplastic material, especially glass or metal (or alloy).

The container may have rigid walls or deformable walls, especially in the form of a tube or a tubular bottle.

The container may comprise means for initiating or facilitating the distribution of the composition. By way of example, the container may have deformable walls so as to allow the composition to exit in response to a positive pressure inside the container, this positive pressure being caused by elastic (or non-elastic) squeezing of the walls of the container.

The container may consist of a carton with a base delimiting at least one housing containing the composition, and a lid, especially articulated on the base, and capable of at least partially covering the base. Such a carton is described, for example, in patent application WO 03/018423 or in patent FR 2 791 042.

The container may be equipped with a drainer arranged in the region of the aperture of the container. Such a drainer makes it possible to wipe the applicator and possibly the rod to which it may be securely fastened. Such a drainer is described, for example, in patent FR 2 792 618.

The content of the patents or patent applications mentioned previously are incorporated into the present patent application by reference.

According to one particular mode of the invention, the assembly may comprise:
a composition A containing at least one phenanthrenol compound and optionally an ingredient for promoting its solubilization and/or its stabilization and/or its activity;
a composition B, conditioned separately from composition A, comprising at least one additional cosmetic or dermatological active agent.

In this case, compositions A and B may be conditioned separately inside two compartments, formed either by two separate containers, or within a single device.

The term “single device” means a device via which the two compartments are fastened together. Such a device may be obtained via a process of moulding the two compartments as a single component, especially made of a thermoplastic material. It may also result from any form of assembling, especially by bonding, welding or other click-fastening.

According to a first embodiment, the two containers are independent of each other. Such containers may be in various forms. They may especially be tubes, bottles or tubs.

One and/or the other of the containers may have mounted thereon a manually actuated pump on which is mounted a push-button for actuating the pump and dispensing the composition via at least one dispensing orifice.

Alternatively, one and/or the other of the containers is pressurized, especially by means of a propellant, in particular a propellant gas. In this case, the container(s) is (are) equipped with a valve on which is mounted a push-button equipped with a nozzle or any other diffusion means for dispensing the product.

The propellant may be admixed with the composition to be dispensed or separate, especially via a piston that can slide inside the container, or via the flexible walls of a bag inside which the composition is placed.

The containers may be constituted by various materials: plastic, glass or metal.

Also alternatively, the two compartments are formed from two concentric compartments formed inside a tube, and mounted on them is a pump without an air return, equipped with a push-button with one or two dispensing orifices. The interior of the tube is provided with a piston which rises in the direction of the pump as and when the compositions within the containers are withdrawn. Such dispensing modes are used especially for dispensing toothpastes.

The invention also relates to a cosmetic process for caring for and/or cleansing and/or making up the skin or its integuments, comprising the application to the skin or its integuments of a composition as defined previously.

According to the invention, the term “integuments” means the skin, the nails, the eyelashes and/or bodily hair, and head hair.

Preferably, it will be a composition for the skin.

In particular, the process according to the invention is directed towards attenuating skin imperfections associated with ageing, in particular actinic ageing.

The expression “skin imperfections associated with ageing of the skin”, in particular actinic ageing, especially means a loss of firmness and/or elasticity and/or tonicity and/or flexibility of the skin, the formation of wrinkles and fine lines, expression wrinkles, in particular on the forehead and between the eyebrows, wrinkles and/or fine lines around the mouth, and/or slackening in the area around the lips, in particular the moustache area (region between the top lip and the nose), a dull complexion, the appearance of browning and/or yellowing of the skin, and/or the appearance of age marks, heterogeneity of the complexion (age marks or actinic lentigo), the appearance and/or visibility of the pores, and the wizened appearance of the skin.

It will especially be intended for people with mature or even very mature skin.

According to the invention, the term “mature skin” especially means the skin of people at least 40 years old.

According to the invention, the term “very mature skin” especially means the skin of people at least 50, in particular at least 60 or even 65 years old.

The compositions and/or combinations according to the invention for preventing and/or smoothing out expression wrinkles will be applied around the orifices constituted by the nose (nasal grooves), the mouth (perioral wrinkles and “bitterness” wrinkles) and the eyes (crow's feet wrinkles), around which are located the skin muscles, and also between the eyebrows (glabella or lion wrinkles) and on the forehead. In particular, it will be sought to prevent and/or smooth out the wrinkles of the forehead and between the eyebrows.

The compositions and/or combinations according to the invention for preventing and/or treating ageing in the area around the lips will be applied in particular to menopausal women, in particular in the moustache area.

The compositions and/or combinations according to the invention for preventing and/or treating the wizened appearance of the skin will be applied especially to the back of the hands.

The compositions and/or combinations according to the invention for reducing the appearance and/or visibility of the pores will be applied in particular to the T area (forehead, nose, cheeks and chin), and especially in the case of asiatic or Caucasian populations.

According to another embodiment, the invention is directed towards attenuating skin imperfections of greasy skin, in particular for making the skin matt, and/or reducing the appearance and/or visibility of the pores, and/or reducing the shiny appearance of the skin, and/or tightening and/or refining the grain of the skin.

Of course, the non-phenanthrenol compounds, etc., described above can be used in any combination of mixtures thereof, each with the other, in formulating compositions according of the invention.

According to the invention, the expression “skin imperfections of greasy skin” means aesthetic disorders such as shiny skin, poor staying power of makeup, skin grain generally associated with a desquamation defect, accentuated visibility of the pores, or skin whose follicular orifices are dilated or filled with tiny horny spicules, or even with comedones or blackheads (resulting, however, more from a phenomenon of retention than from an increase in excretion).

The term “to make matt” means to make the skin visibly more matt or less shiny. The matting effect of the composition may especially be evaluated using a gonioreflectometer, by measuring the ratio R between the specular reflection and the diffuse reflection. A value of R of less than or equal to 2 generally reflects a matting effect.

In particular, the composition is applied to the areas of the face or of the forehead in which the skin is shiny.

Preferably, the composition intended for greasy skin also comprises at least one agent chosen from desquamating agents, depigmenting agents, antioxidants, anti-glycation agents, agents for stimulating the synthesis of dermal and/or epidermal macromolecules and/or for preventing their degradation, calmatives and/or anti-irritant products, sebo-regulating or anti-seborrhoeic agents, and astringents.

According to one particular mode of the invention, the process is intended for caring for individuals with dark skin (especially of phototypes IV to VI).

According to another mode of the invention, the process is intended for caring for individuals with fair skin (especially of phototypes I to III).

The above written description of the invention provides a manner and process of making and using it such that any person skilled in this art is enabled to make and use the same, this enablement being provided in particular for the subject matter of the appended claims, which make up a part of the original description and including a composition comprising, in a physiologically acceptable medium, at least one phenanthrenol compound as described above and at least one non-phenanthrenol compound (such as, e.g., a non-phenanthrenol compound that promotes the solubilization, stabilization and/or activity of the phenanthrenol compound. In addition to the description above, the amount of non-phenanthrenol compound present in the invention compositions ranges from, e.g., 0.1-75% and more. Also fully described and enabled is a composition comprising, in a physiologically acceptable medium, (A) at least one phenanthrenol compound and at least one component selected from the group consisting of (B)-(H):
(B) 5-n-octanoylsalicylic acid, optionally in combination with salicylic acid;
(C) at least one calmative chosen from glycyrrhetinic acid and salts and derivatives thereof, extract of Eperua falcata bark, and a mixture thereof;
(D) at least one desquamating agent and at least one calmative;
(E) at least one sebo-regulating agent chosen from zinc salts, copper derivatives, phthalimidoperoxyhexanoic acid and phthalimidoperoxycaproic acid (PAP), and mixtures thereof;
(F) at least one depigmenting active agent chosen from ascorbyl glucoside (vitamin CG) and ellagic acid, and a mixture thereof;
(G) at least one active agent chosen from an active agent for stimulating the synthesis of glycosaminoglycans, an anti-glycation active agent, and mixtures thereof; and
(H) at least one foaming surfactant.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted. Phrases such as “mention may be made,” etc. preface examples of materials that can be used and do not limit the invention to the specific materials, etc., listed.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. A composition comprising, in a physiologically acceptable medium, (A) at least one phenanthrenol compound and a component selected from the group consisting of (B)-(H):
(B) 5-n-octanoylsalicylic acid, optionally in combination with salicylic acid;
(C) at least one calmative chosen from glycyrrhetinic acid and salts and derivatives thereof, extract of Eperua falcata bark, and a mixture thereof;
(D) at least one desquamating agent and at least one calmative;
(E) at least one sebo-regulating agent chosen from zinc salts, copper derivatives, phthalimidoperoxyhexanoic acid and phthalimidoperoxycaproic acid (PAP), and mixtures thereof;
(F) at least one depigmenting active agent chosen from ascorbyl glucoside (vitamin CG) and ellagic acid, and a mixture thereof;
(G) at least one active agent chosen from an active agent for stimulating the synthesis of glycosaminoglycans, an anti-glycation active agent, and mixtures thereof; and
(H) at least one foaming surfactant.

2. The composition according to claim 1 comprising, in a physiologically acceptable medium, at least one phenanthrenol compound, 5-n-octanoylsalicylic acid, and, optionally, salicylic acid.

3. The composition according to claim 1 comprising, in a physiologically acceptable medium, at least one phenanthrenol compound and at least one calmative chosen from glycyrrhetinic acid and salts and derivatives thereof, extract of Eperua falcata bark, and a mixture thereof.

4. The composition according to claim 1 comprising, in a physiologically acceptable medium, at least one phenanthrenol compound, at least one desquamating agent and at least one calmative.

5. The composition according to claim 1, in which the phenanthrenol compound is formulated in porous polyamide particles.

6. The composition according to claim 1, comprising, in a physiologically acceptable medium, at least one phenanthrenol compound and at least one sebo-regulating agent chosen from zinc salts, copper derivatives, phthalimidoperoxyhexanoic acid and phthalimidoperoxycaproic acid (PAP), and mixtures thereof.

7. The composition according to claim 1, comprising, in a physiologically acceptable medium, at least one phenanthrenol compound and at least one depigmenting active agent chosen from ascorbyl glucoside (vitamin CG) and ellagic acid, and a mixture thereof.

8. The composition according to claim 1, comprising, in a physiologically acceptable medium, at least one phenanthrenol compound and at least one active agent chosen from an active agent for stimulating the synthesis of glycosaminoglycans, an anti-glycation active agent, and mixtures thereof.

9. The composition according to claim 1, comprising, in a physiologically acceptable medium, at least one phenanthrenol compound and at least one foaming surfactant selected form the group consisting of decyl glucoside, a sodium lauryl ether sulfate, an oxyethylenated (7 EO) glyceryl cocoate, and mixtures thereof.

10. A composition according to claim 1 comprising at least one phenanthrenol compound, 5-n-octanoylsalicylic acid, salicylic acid, a potassium salt of glycyrrhetinic acid, and an extract of Eperua falcata, the phenanthrenol compound(s), 5-n-octanoylsalicylic acid and potassium salt of glycyrrhetinic acid being formulated in porous polyamide particles.

11. A composition according to claim 1 comprising a phenanthrenol compound, 5-n-octanoylsalicylic acid, salicylic acid, a potassium salt of glycyrrhetinic acid, an extract of Eperua falcata, and further comprising at least one foaming surfactant.

12. The composition according to claim 1, comprising a desquamating agent formulated in porous polyamide particles.

13. The composition according to claim 1, comprising a calmative formulated in porous polyamide particles.

14. An assembly comprising:
a) a container delimiting at least one compartment, the container being closed by a closing member; and
b) a composition placed inside the compartment, the composition being the composition according to claim 1.

15. A process for reducing cutaneous imperfections of greasy skin, or caring for, or cleansing, or removing makeup from the skin, comprising the topical application to the skin of a composition comprising at least one phenanthrenol compound.

16. The process according to claim 13, wherein the composition further comprises at least one agent chosen from desquamating agents, depigmenting agents, antioxidants, anti-glycation agents, agents for stimulating the synthesis of dermal and/or epidermal macromolecules and/or for preventing their degradation, calmatives and/or anti-irritants, sebo-regulating or anti-seborrhoeic agents, and astringents.

17. The composition according to claim 1, in which the phenanthrenol compound is chosen from the phenanthrenol compounds of formula (I): 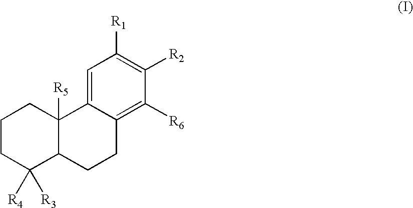
in which:
R₁, R₂ and R₆ denote, independently of each other, a group chosen from —H, —OH, a linear C₁-C₄ alkyl group, a branched C₃-C₄ alkyl group, and a radical —OR with R being a C₁-C₃ alkyl radical, with the proviso that one of the groups R₁, R₂ or R₆ is a group —OH and another one of the groups R₁, R₂ and R₆ is a linear C₁-C₄ alkyl group or a branched C₃-C₄ alkyl group;
R₃ and R₄ denote, independently of each other, a C₁-C₃ alkyl group;
R₅ denotes a C₁-C₆ alkyl group;
and also the stereoisomers and diastereoisomers thereof, and mixtures thereof.

18. The composition according to claim 17, in which:
R₁, R₂ and R₆ are each different and denote, independently of each other, a group chosen from —H, —OH and a branched C₃-C₄ alkyl group;
R₃, R₄ and R₅ denote a methyl group.

19. The composition according to claim 17, in which the phenanthrenol compounds are chosen from:
4-b,5,6,7,8,8a,9,10-octahydro-4-b,8,8-trimethyl-1-(1-methylethyl)-, (4bR,8aR)-rel-2-phenanthrenol;
4-b,5,6,7,8,8a,9,10-octahydro-4-b,8,8-trimethyl-1-(1-methylethyl)-2-phenanthrenol;
4-b,5,6,7,8,8a,9,10-octahydro-4-b,8,8-trimethyl-1-(1-methylethyl)-, (4bS,8aR)-2-phenanthrenol;
4-b,5,6,7,8,8a,9,10-octahydro-4-b,8,8-trimethyl-1-(1-methylethyl)-, (4bS,8aS)-2-phenanthrenol.

20. The composition according to claim 17, in which the phenanthrenol compound has the structure (I′) below: [chemical structure]
